(19) United States
(12) Patent Application Publication
(10) Pub. No.: US 2026/0166140 A1
(43) Pub. Date: Jun. 18, 2026

(54) Combinations Involving Epidermal Growth Factor Receptor Tyrosine Kinase Inhibitors For the Treatment of Cancer

(71) Applicant: AstraZeneca AB, Södertälje (SE)

(72) Inventors: Frank Irvine COMER, Gaithersburg, MD (US); Paul David SMITH, Cambridge (GB); Nicolas FLOCH, Cambridge (GB); Italia GRENGA, Waltham, MA (US); Ryan James HARTMAIER, Waltham, MA (US)

(21) Appl. No.: 18/875,363

(22) PCT Filed: Jun. 26, 2023
(86) PCT No.: PCT/EP2023/067249

§ 371 Date: Dec. 16, 2024

Related U.S. Application Data
(60) Provisional application No. 63/367,068, filed on Jun. 27, 2022.

(51) Int. Cl. A61K 39/395; A61K 31/506; A61K 47/68; A61P 35/00

(52) U.S. Cl. CPC A61K 39/3955; A61K 31/506; A61K 47/68037; A61K 47/6849; A61K 47/6889; A61P 35/00

(57) ABSTRACT

This disclosure relates to an Epidermal Growth Factor Receptor (EGFR) Tyrosine Kinase Inhibitor (TKI) and an anti-EGFR/cMET antibody molecule for use in combination in the treatment of cancer (for example, non-small cell lung cancer [NSCLC]).

### CROSS-REFERENCE TO RELATED APPLICATIONS

This application is a national stage entry application under § 371 of international application number, PCT/EP2023/067249, filed on Jun. 26, 2023, and claims the benefit under 35 U.S.C. § 119(e) of U.S. Provisional Patent Application No. 63/367,068, filed Jun. 27, 2022. The content of each of these applications is incorporated by reference herein in its entirety for all purposes.

### REFERENCE TO SEQUENCE LISTING SUBMITTED ELECTRONICALLY

This application is accompanied by an XML file as a computer-readable format containing the sequence listing entitled, “EGFCM-400-WO-PCT Updated”, created on Mar. 3, 2026, with a file size of 75,687 bytes, the content of which is hereby incorporated by reference in its entirety.

### TECHNICAL FIELD

The specification relates to an Epidermal Growth Factor Receptor (EGFR) Tyrosine Kinase Inhibitor (TKI) for use in the treatment of cancer (for example, non-small cell lung cancer [NSCLC]), wherein the EGFR TKI is for use in combination with anti-EGFR/cMET antibody molecule.

### BACKGROUND

There are multiple drugs directed toward epidermal growth factor receptor (EGFR) that are either approved or in clinical development. For example, two first generation (erlotinib & gefitinib), two second generation (afatinib & dacomitinib) and a third generation (osimertinib) tyrosine kinase inhibitors (TKIs) are currently available for the management of EGFR mutation-positive non-small cell lung cancer (NSCLC). All these TKIs are effective in patients with NSCLC whose tumour harbour the in-frame deletions in exon 19 and the L858R point mutation in exon 21 of EGFR. These two mutations represent approximatively 90% of all EGFR mutations. In approximately 50% of patients, resistance to first and second generation TKI is mediated by the acquisition of the ‘gatekeeper’ mutation T790M. Currently, osimertinib is the only registered EGFR TKI that is active against exon 19 deletions and L858R mutation, regardless of the presence of T790M mutation. However, even patients treated with osimertinib ultimately progress, predominantly due to the development of acquired resistance resulting from other resistance mechanisms. As such, there remains a need to develop new therapies for the treatment of cancer, especially for patients whose disease has progressed following treatment with a third generation EGFR TKI.

cMET, the gene product of the proto-oncogene MET, is a receptor tyrosine kinase expressed primarily on the surface of epithelial cells. Aberrant expression and dysregulation of the cMET pathway has been reported for a wide variety of human cancers, including non-small cell lung, colorectal, gastrointestinal, head and neck, pancreatic, renal, and hepatocellular cancers, among many others (Organ, 2011; Birchmeier, 2003; Mo, 2017; Sierra, 2011). There is a large and growing body of literature demonstrating that there is cross talk and direct interaction between the EGFR and cMET signalling pathways, and that this cross talk functionally translates into resistance to EGFR and cMET targeted therapies in the clinic (McDermott, 2010; Moores, 2016; Suda, 2010).

Antibody drug conjugates (ADCs) have been investigated as a mean of overcoming limitations associated with treatment of cMET and EGFR expressing cancers. One EGFR directed ADC, depatuxizumab mafodotin (ABT-414), is in Phase III clinical development by AbbVie for glioblastoma (Phillips, 2016). ABT-414 was previously tested in Phase II trials for multiple additional solid tumor indications (ClinicalTrials.gov: NCT01741727). The ADC showed limited efficacy at tolerated doses in these indications, and concerning ocular toxicities were frequently observed in treated patients (Tolcher, 2014). A second generation EGFR ADC, ABBV-221 was in clinical development but was discontinued due to safety concerns (Phillips, 2018; Calvo, 2017). There is one cMET targeted ADC, Telisotuzumab Vedotin (ABBV-399), which is entering Phase II clinical development for non-small cell lung cancer (NSCLC) patients whose tumors express high levels of cMET, both as monotherapy and in combination with the EGFR inhibitor, erlotinib (Angevin, 2017; Wang, 2017). The cMET ADC+EGFR TKI combination has shown clinical activity in Phase I trials in this selected patient population, with peripheral neuropathy and skin rash as the most frequent treatment related adverse events (Angevin, 2017; Calvo, 2017).

A bispecific antibody targeting EGFR and cMET has also been developed and is being clinically investigated in the treatment of patients with advanced NSCLC as a monotherapy and in combination with third generation EGFR TKIs (ClinicalTrials.gov: NCT02609776).

The nature of bispecific antibodies allows for fine tuning of the interactions between each target to impact the overall properties of the molecule, which could produce an ADC with an acceptable therapeutic window (Comer, 2018). This concept has been tested for EGFR and cMET in vitro, but investigators have yet to demonstrate proof of concept in vivo of an improvement in safety or efficacy compared to the EGFR and cMET ADC's noted above (Sellmann, 2016).

Accordingly, there is a need for new therapeutic strategies for targeting EGFR and cMET expressing cancers that demonstrate both efficacy and an acceptable safety profile.

### SUMMARY OF THE DISCLOSURE

The inventors recognised that developing a developing anti-EGFR/cMET antibody molecules that bind EGFR with a low affinity (e.g. that binds to human EGFR with a dissociation constant (Kd) that is equal to or higher than 10 nM) could be used in combination with known EGFR TKIs as an effective treatment of cancers (e.g. NSCLCs).

As demonstrated herein, an anti-EGFR/cMET antibody molecule comprising such a low affinity EGFR binding domain conjugated to a drug (an antibody drug conjugate (“ADC”)) displayed reduced on-target toxicity in normal tissues such as skin toxicity and therefore were demonstrated to exhibit an improved safety profile compared to conjugates comprising an EGFR antigen-binding domain that binds human EGFR with a higher affinity.

Furthermore, an ADC comprising this low affinity EGFR binding domain used in combination with the third generation TKI osimertinib was demonstrated to effectively treat a range of EGFR mutant cancer models, including cancer models that had developed resistance to osimertinib. Hence, it is believed that the combination of the antibody molecules disclosed herein and EGFR TKIs may provide a safe and effective therapy against EGFR-associated cancer, e.g. in patients that have developed resistance to EGFR TKIs.

In one aspect, provided herein is an EGFR TKI for use in the treatment of cancer in a human patient, wherein the EGFR TKI is administered in combination with an anti-EGFR/cMET antibody molecule, wherein the anti-EGFR/cMET antibody molecule comprises an EGFR binding domain and a cMET binding domain, wherein the EGFR binding domain comprises:
a. a heavy chain variable (VH) region comprising the following complementarity determining regions (CDRs):
   i. HCDR1 having the amino acid sequence of SEQ ID NO: 1
   ii. HCDR2 having the amino acid sequence of SEQ ID NO: 2
   iii. HCDR3 having the amino acid sequence of SEQ ID NO: 3,
or a variant thereof in which one or two or three amino acids in one or more of HCDR1, HCDR2, or HCDR3 are substituted with another amino acid; and
b. a light chain variable (VL) region comprising the following CDRs:
   i. LCDR1 having the amino acid sequence of SEQ ID NO: 4
   ii. LCDR2 having the amino acid sequence of SEQ ID NO: 5
   iii. LCDR3 having the amino acid sequence of SEQ ID NO: 6, or a variant thereof in which one or two or three amino acids in one or more of HCDR1, HCDR2, or HCDR3 are substituted with another amino acid.

In another aspect, provided herein is an anti-EGFR/cMET antibody molecule for use in the treatment of cancer in a human patient, wherein the anti-EGFR/cMET antibody molecule is administered in combination with an EGFR TKI, wherein the anti-EGFR/cMET antibody molecule comprises an EGFR binding domain and a cMET binding domain, wherein the EGFR binding domain comprises:
a. a heavy chain variable (VH) region comprising the following complementarity determining regions (CDRs):
   i. HCDR1 having the amino acid sequence of SEQ ID NO: 1
   ii. HCDR2 having the amino acid sequence of SEQ ID NO: 2
   iii. HCDR3 having the amino acid sequence of SEQ ID NO: 3,
or a variant thereof in which one or two or three amino acids in one or more of HCDR1, HCDR2, or HCDR3 are substituted with another amino acid; and
b. a light chain variable (VL) region comprising the following CDRs:
   i. LCDR1 having the amino acid sequence of SEQ ID NO: 4
   ii. LCDR2 having the amino acid sequence of SEQ ID NO: 5
   iii. LCDR3 having the amino acid sequence of SEQ ID NO: 6,
or a variant thereof in which one or two or three amino acids in one or more of HCDR1, HCDR2, or HCDR3 are substituted with another amino acid.

In another aspect, provided herein is a method of treating cancer in a human patient comprising administering an anti-EGFR/cMET antibody molecule in combination with an EGFR TKI, wherein the anti-EGFR/cMET antibody molecule comprises an EGFR binding domain and a cMET binding domain, wherein the EGFR binding domain comprises:
a. a heavy chain variable (VH) region comprising the following complementarity determining regions (CDRs):
   i. HCDR1 having the amino acid sequence of SEQ ID NO: 1
   ii. HCDR2 having the amino acid sequence of SEQ ID NO: 2
   iii. HCDR3 having the amino acid sequence of SEQ ID NO: 3,
or a variant thereof in which one or two or three amino acids in one or more of HCDR1, HCDR2, or HCDR3 are substituted with another amino acid; and
b. a light chain variable (VL) region comprising the following CDRs:
   i. LCDR1 having the amino acid sequence of SEQ ID NO: 4
   ii. LCDR2 having the amino acid sequence of SEQ ID NO: 5
   iii. LCDR3 having the amino acid sequence of SEQ ID NO: 6,
or a variant thereof in which one or two or three amino acids in one or more of HCDR1, HCDR2, or HCDR3 are substituted with another amino acid.

In another aspect, provided herein is a pharmaceutical combination of an EGFR/cMET antibody molecule and an EGFR TKI, wherein the anti-EGFR/cMET antibody molecule comprises an EGFR binding domain and a cMET binding domain, wherein the EGFR binding domain comprises:
a. a heavy chain variable (VH) region comprising the following complementarity determining regions (CDRs):
   i. HCDR1 having the amino acid sequence of SEQ ID NO: 1
   ii. HCDR2 having the amino acid sequence of SEQ ID NO: 2
   iii. HCDR3 having the amino acid sequence of SEQ ID NO: 3,
or a variant thereof in which one or two or three amino acids in one or more of HCDR1, HCDR2, or HCDR3 are substituted with another amino acid; and
b. a light chain variable (VL) region comprising the following CDRs:
   i. LCDR1 having the amino acid sequence of SEQ ID NO: 4
   ii. LCDR2 having the amino acid sequence of SEQ ID NO: 5
   iii. LCDR3 having the amino acid sequence of SEQ ID NO: 6,
or a variant thereof in which one or two or three amino acids in one or more of LCDR1, LCDR2, or LCDR3 are substituted with another amino acid.

In some instances, administration of the EGFR TKI and the anti-EGFR/cMET antibody molecule is separate, sequential, or simultaneous.

Examples of suitable EGFR TKIs for use in combination treatments claimed are described. In some instances, the EGFR TKI is osimertinib or a pharmaceutically acceptable salt thereof.

In some instances, the anti-EGFR binding domain comprises HCDR1 having the amino acid sequence of SEQ ID NO: 1, HCDR2 having the amino acid sequence of SEQ ID NO: 2, HCDR3 having the amino acid sequence of SEQ ID NO: 3, LCDR1 having the amino acid sequence of SEQ ID NO: 4, LCDR2 having the amino acid sequence of SEQ ID NO: 5, and LCDR3 having the amino acid sequence of SEQ ID NO: 6. In some instances, the anti-EGFR binding domain comprises a VH region comprising an amino acid sequence having at least 70%, at least 80%, at least 90%, or at least 95% sequence identity to the amino acid sequence of SEQ ID NO: 16; and a VL region comprising an amino acid sequence having at least 70%, at least 80%, at least 90%, or at least 95% sequence identity to the amino acid sequence of SEQ ID NO: 20.

In some instances, the anti-cMET binding domain comprises:
a. a heavy chain variable (VH) region comprising the following complementarity determining regions (CDRs):
   i. HCDR1 having the amino acid sequence of SEQ ID NO: 24
   ii. HCDR2 having the amino acid sequence of SEQ ID NO: 25
   iii. HCDR3 having the amino acid sequence of SEQ ID NO: 26,
or a variant thereof in which one or two or three amino acids in one or more of HCDR1, HCDR2, or HCDR3 are substituted with another amino acid; and
b. a light chain variable (VL) region comprising the following CDRs:
   i. LCDR1 having the amino acid sequence of SEQ ID NO: 27
   ii. LCDR2 having the amino acid sequence of SEQ ID NO: 28
   iii. LCDR3 having the amino acid sequence of SEQ ID NO: 29,
or a variant thereof in which one or two or three amino acids in one or more of HCDR1, HCDR2, or HCDR3 are substituted with another amino acid.

In some instances, the anti-cMET binding domain comprises HCDR1 having the amino acid sequence of SEQ ID NO: 24, HCDR2 having the amino acid sequence of SEQ ID NO: 25, HCDR3 having the amino acid sequence of SEQ ID NO: 26, LCDR1 having the amino acid sequence of SEQ ID NO: 27, LCDR2 having the amino acid sequence of SEQ ID NO: 28, and LCDR3 having the amino acid sequence of SEQ ID NO: 29. In some instances, the anti-cMET binding domain comprises a VH region comprising an amino acid sequence having at least 70%, at least 80%, at least 90%, or at least 95% sequence identity to the amino acid sequence of SEQ ID NO: 38; and a VL region comprising an amino acid sequence having at least 70%, at least 80%, at least 90%, or at least 95% sequence identity to the amino acid sequence of SEQ ID NO: 40.

In some instances, the antibody molecule is conjugated to a drug. The drug may comprise a cytotoxin, a radioisotope, an immunomodulator, a cytokine, a lymphokine, a chemokine, a growth factor, a tumor necrosis factor, a hormone, a hormone antagonist, an enzyme, an oligonucleotide, a DNA, an RNA, an siRNA, an RNAi, a microRNA, a photoactive therapeutic agent, an anti-angiogenic agent, a pro-apoptotic agent, a peptide, a lipid, a carbohydrate, a chelating agent, or combinations thereof. In some instances, the drug is a topoisomerase I inhibitor as further described herein.

In some instances, the cancer is non-small cell lung cancer (NSCLC). In some instances, the non-small cell lung cancer is an EGFR mutation-positive NSCLC. EGFR mutation-positive NSCLCs include those comprising activation mutations such as a L858R mutation and/or one or more deletions in exon 19 in the EGFR gene, as well as mutations associated with EGFR TKI resistance such as an insertion in exon 20 in the EGFR gene. As demonstrated herein, combination of EGFR-cMET conjugates and osimertinib showed efficacy across a range of EGFR mutant cancers, including those that are classed as osimertinib resistant.

### BRIEF DESCRIPTION OF DRAWINGS

Instances and experiments illustrating the principles of the disclosure will now be discussed with reference to the accompanying figures in which:

FIG. 1A. Graphical depiction of RAA22/B09-57 DuetMab. Shown are the Fabs of anti-EGFR RAA22, Fab of anti-cMET B09-57 and Hole and Knob heavy chains. The structural rendering is a composite of individual domain structures.

FIG. 1B. Graphical depiction of QD6/B09-57 DuetMab. Shown are the Fabs of anti-EGFR QD6, Fab of anti-cMET B09-57 and Hole and Knob heavy chains. The structural rendering is a composite of individual domain structures.

FIG. 2. Concurrent binding studies using antigen capture format were performed by Octet analysis. Sensors loaded with human cMET antigen were exposed to successive association and dissociation interactions first with antibodies then with human EGFR antigen. Ass=association; Diss=dissociation; NI-NTA=Nickel-nitrilotriacetic acid.

FIG. 3A. ELISA results showing EGFR and cMet species cross reactivity. The high affinity monospecific EGFR IgG, QD6, as well as the monovalent bispecific EGFR/cMET DuetMAb, QD6/B09, bound to human, cynomolgus monkey, and mouse EGFR. The lowered affinity monospecific EGFR IgG, RAA22, bound more weakly to human, cynomolgus monkey, and mouse EGFR compared to QD6 and the corresponding monovalent bispecific EGFR/c-Met DuetMAb, RAA22/B09, showed still weaker binding to human and cynomolgus monkey EGFR relative to the bivalent parental IgG, RAA22, and nominal binding to mouse EGFR. The monospecific c-Met IgG, B09, as well as all of the bispecific variants, showed comparable binding to human and cynomolgus monkey c-Met but no detectable binding to mouse c-Met.

FIG. 3B ELISA results showing EGFR and cMet family specificity. None of the antibodies tested showed any appreciable binding to any of the EGFR HER family proteins (HER2, HER3, or HER4) or any of the c-Met family members (Ron (CD136) or Semaphorin 3a).

FIG. 4A. Internalization profiles of QD6/B09 DuetMab and its respective single-arm control antibodies. Internalization profiles are displayed via time course of the respective membrane and cytoplasm signals for each construct. QD6/B09 set was acquired using an Opera confocal fluorescence microscope.

FIG. 4B. Internalization profiles of RAA22/B09 DuetMab and its respective single-arm control antibodies. Internalization profiles are displayed via time course of the respective membrane and cytoplasm signals for each construct. The set was acquired using a Zeiss spinning-disc confocal fluorescence microscope.

Identical profiles for QD6/B09 and QD6/IgG indicate internalization mode driven by EGFR-arm of QD6/B09 DuetMab, whereas RAA22/B09 DuetMab requires engagement of both EGFR and c-MET arms for efficient internalization.

FIG. 5A. Internalization profile of RAA22/B09-AZD1508 ADC in cells expressing moderate and high target c-MET and EGFR cell surface receptors. Shown are membrane, cytoplasm and total signals for RAA22/B09-AF647 ADC in H1975 cells. One representative experiment of 2 is shown. H1975 cells show concurrent drop in total and membrane intensities indicating dissociation of antibody from the cell surface.

FIG. 5B is equivalent to FIG. 8A but in HCC827 cells. HCC827 cells have stable total signal over experimental time course. Decrease of membrane signal is derived antibody internalization.

FIG. 6A. Internalization of RAA22/B09-AZD1508 ADC single arm control antibodies in HCC827 cells. Intensity profiles of the RAA22/IgG single arm is ˜10 fold lower than RAA22/B09 due to weaker binding to EGFR through single arm binding FIG. 6B B09/IgG single arm dissociates from cell membrane as signaled by simultaneous drop in total and membrane signals over time.

FIG. 7. Analysis of the relative contribution of the individual antibody arms to the cytotoxic activity of the bispecific ADC. NCI H1975 cells were pretreated with an excess of unarmed parental antibodies to block either EGFR or cMET. Next, EGFR-cMET ADC (RAA22/B09-AZ1508) was added to cells in a 4× serial dilution series with a final concentration ranging from 67 nM down to 0.0009 nM. The treated cells were cultured for 72 hours in a humidified incubator at 37° C. and 5% CO₂. The metabolic activity was determined using CellTiter-Glo Luminescent Viability Assay (Promega). Data were plotted as percent metabolic activity relative to untreated control. IC50 values were determined using logistic non-linear regression analysis between the maximal viability (untreated cells) and the maximal response (peak inhibition) with GraphPad Prism software.

FIG. 8. Further evaluation of the individual antibody arms to the activity of the bispecific ADC. Monospecific, monovalent ADCs were constructed by pairing each binding arm with a non-binding isotype control arm (R347) to produce EGFR ADC (RAA22/R347-AZ1508) and anti-cMET ADC (B09/R347-AZ1508), The ADCs were added to NCI H1975 cells in a 4× serial dilution series with a final concentration ranging from 67 nM down to 0.0009 nM. Percent metabolic activity was determined as described in FIG. 7.

FIG. 9A. Mouse PDX trials were carried out to determine the efficacy of high affinity (QD6/B09-AZ1508) and low affinity (RAA22/B09-AZ1508) EGFR-cMET ADCs in a large number of patient derived xenograft (PDX) models of human cancer in immunodeficient mice. Each compound was tested at a single dose level of 3 mg/kg in a single mouse for each PDX model representing a different human tumor. Percent tumor growth relative to untreated control tumors (% T/C) was calculated for tumors that grew larger than the initial volume and percent tumor regression was calculated for tumors that showed a reduction in size compared to the initial tumor volume. (A) shows the direct comparison of the high and low affinity ADCs in each model and

FIG. 9B shows waterfall plots for the high affinity ADC in rank order of efficacy.

FIG. 9C shows waterfall plots for the low affinity ADC in rank order of efficacy.

FIG. 10. Dose range finding in vivo efficacy studies in PDX models were carried out in athymic nude mice implanted unilaterally on the flank with tumor fragments harvested from host animals. The high affinity EGFR-cMET ADC, QD6/B09-AZ1508, was tested at dose levels of 1 and 2 mg/kg and the variant with lowered affinity for EGFR, RAA22/B09-AZ1508, was tested at 1, 2, and 3 mg/kg, as indicated in the figure. Tumor volume measurements were taken twice weekly following the initiation of dosing and plotted as line graphs of tumor volume over time. Error bars represent standard error of the means (SEM) and the inset images show the immunohistochemical staining of EGFR and cMET for each model, from tumor tissue taken from an earlier passage of the model.

FIG. 11A In vivo efficacy of EGFR-cMET bispecific ADCs in a subcutaneous and orthotopic Pancreatic PDX model. A) In vivo efficacy of the High Affinity QD6/B09 ADC in the subcutaneous MEDI-PANC-08 PDX model, ●—Untreated, ▪—R347-AZ1508 (3 mg/kg-Q1W×4), ▴—QD6/B09-1508 (1 mg/kg-Q1W×4), ┌—QD6/B09-1508 (2 mg/kg-Q1W×4) and [symbol] —QD6/B09-1508 (3 mg/kg-Q1W×4). Treatment days are indicated by a tick on the x axis, tumor volumes were measure twice a week.

FIG. 11B In vivo efficacy of the Low Affinity RAA2/B09 ADC in the subcutaneous MEDI-PANC-08 PDX model. ●—Untreated, ▪—R347-AZ1508 (3 mg/kg-Q1W×4), ▴—RAA2/B09-1508 (1 mg/kg-Q1W×4), ▾—RAA2/B09-1508 (2 mg/kg-Q1W×4) and [symbol] —RAA2/B09-1508 (3 mg/kg—Q1W×4). Treatment days are indicated by a Tick on the x-axis, tumor volumes were measure twice a week.

FIG. 11C Luciferase imaging of the orthotopic MEDI-PANC-08^{LUC} (luciferase expressing) PDX model. Mice were imaged weekly using the IVIS Spectrum In vivo Imaging system. The images are normalized across all groups and timepoints with the radiance scale (Avg Radiance [p/s/cm2/sr]) set between the max signal (Day 21 Control Gp) and background.

FIG. 11D In vivo efficacy of the Low Affinity RAA2/B09 ADC in the subcutaneous MEDI-PANC-08 PDX model. ●—Untreated, ▪—Gemcitabine (75 mg/kg-Q3/4D×5), ▴ R347-AZ1508 (3 mg/kg—Q1W×4), ▾—RAA2/B09-1508 (2 mg/kg-Q1W×4) and [symbol] —RAA2/B09-1508 (3 mg/kg-Q1W×4). Treatment days are indicated by the arrows, tumor volumes were measured twice a week. The data displayed in FIGS. 11A and B are the group mean tumor volume (mm³)±SEM, in FIG. 11D group mean Radiance [p/s/cm²/sr]±SEM.

FIG. 12A Mean concentrations-time profiles and mean NCA PK parameters for RAA22/B09-57-AZ1508 in Mice. The target compound concentration and the total antibody concentration were measured with an immuno capture LC-MS/MS assay.

FIG. 12B Mean concentrations-time profiles and mean NCA PK parameters for QD6/B09-57-AZ1508 in Mice. The target compound concentration and the total antibody concentration were measured with an immuno capture LC-MS/MS assay.

FIG. 13A Mean concentrations-time profiles and mean NCA PK parameters for RAA22/B09-57-AZ1508 and in Monkeys.

FIG. 13B Mean concentrations-time profiles and mean NCA PK parameters for QD6/B09-57-AZ1508 and in Monkeys.

PK profiles and NCA PK parameters for QD6/B09-57-AZ1508 at 3 mg/kg in 20067312 was based on PK data following second dose.

FIG. 13C is a table summarizing the PK parameters of the molecules

FIG. 14 EGFR-cMET Maia Topoi ADC was evaluated in patient derived xenograft (PDX) models representing multiple types of human cancer in immunodeficient mice as a PDX trial. Compound was tested at a dose level of 10 mg/kg in a single mouse for each PDX model representing a unique human tumor. Percent tumor growth relative to untreated control tumors (% T/C) was calculated for tumors that grew larger than the initial volume (Tumor growth inhibition (% TGI) was defined as Percent stumor growth versus Day 0 between treatment (TX) and control (C) groups, according to the formula: % TGI=1−(TXfinal−TXinitial)/(Cfinal−Cinitial)). and percent tumor regression was calculated for tumors that showed a reduction in size compared to the initial tumor volume (Percent Tumor Regression was defined as the percentage tumor reduction of tumors in treated animals relative to the Day 0 tumor volume (day of initial dose), calculated at study endpoint according to the following formula: % Regression=(TXfinal avg−TXinitial avg)/(TXinitial avg)×100).

FIG. 15 EGFR-cMET Topoi™ ADC was evaluated in patient derived xenograft (PDX) models representing multiple types of human cancer in immunodeficient mice as a PDX trial. Compound was tested at a dose level of 5 mg/kg in a single mouse for each PDX model representing a unique human tumor. Percent tumor growth relative to untreated control tumors (% T/C) was calculated for tumors that grew larger than the initial volume (Tumor growth inhibition (% TGI) was defined as Percent stumor growth versus Day 0 between treatment (TX) and control (C) groups, according to the formula: % TGI=1−(TXfinal−TXinitial)/(Cfinal−Cinitial)). and percent tumor regression was calculated for tumors that showed a reduction in size compared to the initial tumor volume (Percent Tumor Regression was defined as the percentage tumor reduction of tumors in treated animals relative to the Day 0 tumor volume (day of initial dose), calculated at study endpoint according to the following formula: % Regression=(TXfinal avg−TXinitial avg)/(TXinitial avg)×100).

FIG. 16A Two different EGFR-cMET ADCs with different IgG Fc formats (Maia and TM) were evaluated for comparability in the PDX model SQHN-02. The ADCs were tested at 3 dose levels: 2.5, 5, and 10 mg/kg and tumor growth was compared against untreated control animals. A total of 10 animals were treated per treatment and control group.

FIG. 16B Two different EGFR-cMET ADCs with different IgG Fc formats (Maia and TM) were evaluated for comparability in the PDX model Panc-08. The ADCs were tested at 3 dose levels: 2.5, 5, and 10 mg/kg and tumor growth was compared against untreated control animals. A total of 10 animals were treated per treatment and control group.

FIG. 17 depicts the results of the non-small cell lung cancer NSCLC PDX models from FIG. 14 above, highlighting the EGFR mutation status and histology, where known.

FIG. 18 The pharmacokinetic profiles of EGFR-cMET bispecific antibodies INT-009 (RAA22/B09-Maia naked mAb) and INT-009-SG3932 DAR8 ADC (“MAIA ADC”) were compared to B09/RAA2-IgG1-TM mirror mAb (INT-017) and TM-mirror-SG3932 DAR6 ADC (“TM ADC”) in NOD-SCID mice at therapeutic doses of 5 mg/kg.

FIG. 19A depicts results of ADC efficacy in EGFR mutant PDX model ‘LUN487’ containing the L858R EGFR mutation.

FIG. 19B depicts results of ADC efficacy in combination with the 3^{rd} gen TKI osimertinib (‘Osi’) in EGFR mutant PDX model ‘LUN487’ containing the L858R EGFR mutation.

FIG. 19C depicts results of ADC efficacy in EGFR mutant PDX model ‘LUN439’ containing the L858R EGFR mutation.

FIG. 19D depicts results of ADC efficacy in combination with the 3^{rd} gen TKI osimertinib (‘Osi’) in EGFR mutant PDX model ‘LUN439’ containing the L858R EGFR mutation.

FIG. 20A depicts results of ADC efficacy in in EGFR mutant PDX model ‘CTG-2992’ containing an EGFR exon 20 insertion (primary osimertinib resistance)

FIG. 20B depicts results of ADC efficacy in combination with the 3^{rd} gen TKI osimertinib in EGFR mutant PDX model ‘CTG-2992’ containing an EGFR exon 20 insertion (primary osimertinib resistance)

FIG. 21A depicts results of ADC efficacy in EGFR mutant PDX model ‘CTG-2803’ (acquired osimertinib resistance)

FIG. 21B depicts results of ADC efficacy in combination with the 3^{rd} gen TKI osimertinib in EGFR mutant PDX model ‘CTG-2803’ (acquired osimertinib resistance)

FIG. 22A shows a waterfall plot of multiple EGFRmut NSCLC patient-derived xenograft model responses to treatment with 25 mg/kg osimertinib. The x-axis depicts the best response from baseline over the duration of the study. The Y-axis intercept line shows 30% regression from baseline, which define a response

FIG. 22B shows a waterfall plot of multiple EGFRmut NSCLC patient-derived xenograft model responses to treatment with 2 mg/kg EGFR-cMET™ ADC. The x-axis depicts the best response from baseline over the duration of the study. The Y-axis intercept line shows 30% regression from baseline, which define a response

FIG. 22C shows a waterfall plot of multiple EGFRmut NSCLC patient-derived xenograft model responses to treatment with the combination of 25 mg/kg osimertinib and 2 mg/kg EGFR-cMET™ ADC. The x-axis depicts the best response from baseline over the duration of the study. The Y-axis intercept line shows 30% regression from baseline, which define a response

### DETAILED DESCRIPTION

Aspects and instances of the present disclosure will now be discussed with reference to the accompanying figures. Further aspects and disclosure will be apparent to those skilled in the art. All documents mentioned in this text are incorporated herein by reference.

### Targets

### EGFR

Human EGFR (also known as proto-oncogene c-ErbB-1, receptor tyrosine-protein kinase erbB-1 and EC 2.7.10.1) is the protein identified by UniProt P00533. Alternative splicing of mRNA encoded by the human EGFR gene (also known as ERBB, ERBB1 and HER1) yields four isoforms: isoform 1 (UniProt: P00533-1, v2 (last sequence update: Nov. 1, 1997)); isoform 2 (UniProt: P00533-2, v1), which comprises the substitutions F404L and L405S relative to isoform 1, and which lacks the amino acid sequence corresponding to positions 406 to 1210 of isoform 1; isoform 3 (UniProt: P00533-3, v1), which comprises substitutions at position 628 to 705 of isoform 1, and which lacks the amino acid sequence corresponding to positions 706 to 1210 of isoform 1; and isoform 4 (UniProt: P00533-4), which comprises the substitution C628S relative to isoform 1, and which lacks the amino acid sequence corresponding to positions 629 to 1210 of isoform 1.

The structure and function of EGFR is reviewed e.g. in Ferguson, Annu Rev Biophys. (2008) 37: 353-373. EGFR is a transmembrane protein that is a receptor for members of the epidermal growth factor family (EGF family). The receptor comprises a large extracellular region, a single spanning transmembrane domain, an intracellular juxtamembrane domain, a tyrosine kinase domain and a C-terminal regulatory region. Binding of EGFR to a ligand induces receptor dimerization and autophosphorylation of several tyrosine residues (Y992, Y1045, Y1068, Y1148 and Y1173) in the C-terminal regulatory region of EGFR.

Aberrant EGFR expression/activity is implicated in many diseases, including nervous system disorders and many cancers.

In this specification “EGFR” refers to EGFR from any species and includes EGFR isoforms, fragments, variants or homologues from any species.

cMET

Human cMET (also known as c-Met, Hepatocyte growth factor receptor (HGFR) or tyrosine-protein kinase Met) is the protein identified by UniProt P08581. Alternative splicing of mRNA encoded by the human MET gene yields three isoforms: isoform 1 (UniProt: P08581-1, v4 (last sequence update: Jul. 7, 2009)); isoform 2 (UniProt: P08581-2), in which the amino acid sequence “STWWKEPLNIVSFLFCFAS” is inserted at position 755 of isoform 1; and isoform 3 (UniProt: P08581-3) also known as Soluble met variant 4, in which the amino acid sequence corresponding to positions 755 to 764 of isoform 1 are substituted with “RHVNIALIQR” and which further lacks the amino acid sequence corresponding to positions 765 to 1390 of isoform 1.

The structure of cMET is reviewed e.g. in Gherardi, 2003, which is herein incorporated by reference in its entirety. cMET is a heterodimer made of an alpha chain (50 kDa) and a beta chain (145 kDa), which are disulphide linked. cMET comprises a N-terminal Sema domain, which mediates binding to hepatocyte growth factor (HGF) and an intracellular kinase domain. Ligand binding at the cell surface induces autophosphorylation of cMET on its intracellular domain that provides docking sites for downstream signalling molecules and the activation of several signalling cascades.

cMET is expressed in normal tissues on the surface of epithelial cells. cMET overexpression is observed in many human tumors and cancers, which is frequently associated with a metastatic phenotype and poor prognosis. Examples of cancers where high levels of cMET expression has been observed includes non-small cell lung cancer (NSCLC), pancreatic cancer, colorectal cancer, head and neck squamous cell carcinoma, breast cancer and esophageal-gastric cancer. In these cancers, co-expression of EGFR and cMET is often observed.

### Antibody Molecules

The present disclosure provides antibody molecules. Antibody molecules according to the present disclosure may be provided in isolated form, in the sense of being free from contaminants, such as antibodies able to bind other polypeptides and/or serum components.

The term “antibody molecule” describes an immunoglobulin whether natural or partly or wholly synthetically produced. The antibody molecule may be human or humanised. The antibody molecule may be a monoclonal antibody molecule. Examples of antibodies are the immunoglobulin isotypes, such as immunoglobulin G (IgG), and their isotypic subclasses, such as IgG1, IgG2, IgG3 and IgG4, as well as fragments thereof.

The term “antibody molecule”, as used herein, thus includes antibody fragments, as long as they display binding to the relevant target molecule(s). Examples of antibody fragments include Fv, scFv, Fab, scFab, F(ab′)₂, Fab2, diabodies, triabodies, scFv-Fc, minibodies and single domain antibodies (e.g. VhH), etc.). Unless the context requires otherwise, the term “antibody molecule”, as used herein, is thus equivalent to “antibody molecule or fragment thereof”.

Antibody molecules and methods for their construction and use are well-known in the art and are described in, for example, Holliger & Hudson, Nature Biotechnology 23(9):1126-1136 (2005). It is possible to take monoclonal and other antibody molecules and use techniques of recombinant DNA technology to produce other antibody or chimeric molecules which retain the specificity of the original antibody. Such techniques may involve introducing CDRs or variable regions of one antibody molecule into a different antibody molecule (EP-A-184187, GB 2188638A and EP-A-239400).

In view of today's techniques in relation to monoclonal antibody technology, antibody molecules can be prepared to most antigens. The antigen-binding domain may be a part of an antibody (for example a Fab fragment) or a synthetic antibody fragment (for example a single chain Fv fragment (ScFv)). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger, 1988.

Antibody molecules according to the present disclosure comprise an antigen-binding domain (also termed herein a “binding domain”). An “antigen-binding domain” or “binding domain” describes the part of a molecule that binds to all or part of the target antigen. Where an antigen is large, an antibody may only bind to a particular part of the antigen, which part is termed an epitope. An antibody antigen-binding site may be provided by one or more antibody variable domains. An antibody antigen-binding site optionally comprises a variable light (VL) region and variable heavy (VH) region. The VH and VL region of an antigen-binding domain together constitute the Fv region.

An antigen-binding domain generally comprises six complementarity-determining regions (CDRs); three in the VH region: HCDR1, HCDR2 and HCDR3, and three in the VL region: LCDR1, LCDR2, and LCDR3. The six CDRs together define the paratope of the antigen-binding domain, which is the part of the antigen-binding domain which binds to the target antigen.

The VH region and VL region comprise framework regions (FRs) either side of each CDR, which provide a scaffold for the CDRs. From N-terminus to C-terminus, VH regions comprise the following structure: N term-[HFR1]-[HCDR1]-[HFR2]-[HCDR2]-[HFR3]-[HCDR3]-[HFR4]-C term; and VL regions comprise the following structure: N term-[LFR1]-[LCDR1]-[LFR2]-[LCDR2]-[LFR3]-[LCDR3]-[LFR4]-C term.

There are several different conventions for defining antibody CDRs and FRs, such as those described in Kabat, 1991, Chothia, 1987, IMGT numbering as described in LeFranc, 2015, and VBASE2, as described in Retter, 2005. The CDRs and FRs of the VH regions and VL regions of the antibody molecules described herein were defined according to Kabat (Kabat, 1991).

Antibody molecules that comprise at least two antigen-binding domains, each of which being capable of binding to a different target may be termed “bispecific antibody molecules”. In contrast, antibody molecules that only bind a single target (e.g. EGFR or cMET) are termed “monospecific antibody molecules”. The present disclosure relates to a bispecific antibody molecule that comprises an EGFR binding domain and a cMET binding domain.

### Anti-EGFR Binding Domains

The binding domain that binds EGFR (anti-EGFR binding domain) typically comprises the CDRs of an antibody molecule which is capable of binding to EGFR. In some instances, the binding domain that binds EGFR additionally comprises the FRs of an antibody molecule which is capable of binding to EGFR. That is, in some instances the binding domain that binds EGFR comprises the VH region and the VL region of an antibody molecule which is capable of binding to EGFR.

In some instances the binding domain that binds EGFR comprises a VH region and a VL region which is, or which is derived from, the VH/VL region of an EGFR-binding antibody clone described herein (i.e. anti-EGFR antibody clones RAA22 or QD6). In some instances, the binding domain that binds EGFR comprises a VH region and a VL region which is, or which is derived from, the VHNL region of RAA22.

In some instances, the binding domain that binds EGFR comprises the three HCDRs or three LCDRs, optionally the three VH CDRs and the three VL CDRs, of anti-EGFR antibody clones RAA22 or QD6, optionally RAA22. The VH and VL domain sequences of antibodies RAA22 and QD6 are described herein, and the three VH and three VL domain CDRs of said antibodies may thus be determined from said sequences.

In some instances, the binding domain that binds EGFR comprises a VH region according to (1) or (2) below:
(1) a VH region comprising the following CDRs:
   HCDR1 having the amino acid sequence of SEQ ID NO: 1
   HCDR2 having the amino acid sequence of SEQ ID NO: 2
   HCDR3 having the amino acid sequence of SEQ ID NO: 3,
   or a variant thereof in which one or two or three amino acids in one or more of HCDR1, HCDR2, or HCDR3 are substituted with another amino acid, or
(2) a VH region comprising the following CDRs:
   HCDR1 having the amino acid sequence of SEQ ID NO: 1
   HCDR2 having the amino acid sequence of SEQ ID NO: 7
   HCDR3 having the amino acid sequence of SEQ ID NO: 3,
   or a variant thereof in which one or two or three amino acids in one or more of HCDR1, HCDR2, or HCDR3 are substituted with another amino acid.

In some instances, the binding domain that binds EGFR comprises a VH region according to (1) above.

In some instances, the binding domain that binds EGFR comprises a VH region according to (1) or (2) above, wherein the VH region additionally comprises the FRs according to (3) below:
(3) HFR1 having the amino acid sequence of SEQ ID NO: 8
   HFR2 having the amino acid sequence of SEQ ID NO: 9
   HFR3 having the amino acid sequence of SEQ ID NO: 10
   HFR4 having the amino acid sequence of SEQ ID NO: 11,
   or a variant thereof in which one or two or three amino acids in one or more of HFR1, HFR2, HFR3, or HFR4 are substituted with another amino acid.

In some instances the binding domain that binds EGFR comprises a VH region comprising the CDRs according to (1) or (2) above, and the FRs according to (3) above.

In some instances the binding domain that binds EGFR comprises a VH region according to (4) or (5) below:
(4) a VH region comprising the CDRs according to (1) and the FRs according to (3),
(5) a VH region comprising the CDRs according to (2) and the FRs according to (3).

In some instances, the binding domain that binds EGFR comprises a VH region according to (4) above.

In some instances the binding domain that binds EGFR comprises a VH region according to (6) or (7) below:
(6) a VH region comprising an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 16.
(7) a VH region comprising an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 18.

In some instances, the binding domain that binds EGFR comprises a VH region according to (6) above.

In some instances the binding domain that binds EGFR comprises a VL region according to (8) or (9) below:
(8) a VL region comprising the following CDRs:
   LCDR1 having the amino acid sequence of SEQ ID NO: 4
   LCDR2 having the amino acid sequence of SEQ ID NO: 5
   LCDR3 having the amino acid sequence of SEQ ID NO: 6,
   or a variant thereof in which one or two or three amino acids in one or more of LCDR1, LCDR2, or LCDR3 are substituted with another amino acid.
(9) a VL region comprising the following CDRs:
   LCDR1 having the amino acid sequence of SEQ ID NO: 4
   LCDR2 having the amino acid sequence of SEQ ID NO: 66
   LCDR3 having the amino acid sequence of SEQ ID NO: 67,
   or a variant thereof in which one or two or three amino acids in one or more of LCDR1, LCDR2, or LCDR3 are substituted with another amino acid.

In some instances, the binding domain that binds EGFR comprises a VL region according to (8) above.

In some instances, the binding domain that binds EGFR comprises a VL region according to (8) or (9) above, wherein the VL region additionally comprises the FRs according to (10) below:
(10) LFR1 having the amino acid sequence of SEQ ID NO: 12
   LFR2 having the amino acid sequence of SEQ ID NO: 13
   LFR3 having the amino acid sequence of SEQ ID NO: 14
   LFR4 having the amino acid sequence of SEQ ID NO: 15,
   or a variant thereof in which one or two or three amino acids in one or more of LFR1, LFR2, LFR3, or LFR4 are substituted with another amino acid.

In some instances the binding domain that binds EGFR comprises a VL region comprising the CDRs according to (8) or (9) above, and the FRs according to (10) above.

In some instances the binding domain that binds EGFR comprises a VL region according to (11) or (12) below:
(11) a VL region comprising the CDRs according to (8) and the FRs according to (10).
(12) a VL region comprising the CDRs according to (9) and the FRs according to (10).

In some instances, the binding domain that binds EGFR comprises a VL region according to (11) above.

In some instances the binding domain that binds EGFR comprises a VL region according to (13) or (14) below:
(13) a VL region comprising an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 20.
(14) a VL region comprising an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 22.

In some instances, the binding domain that binds EGFR comprises a VL region according to (13) above.

In some instances the binding domain that binds EGFR comprises a VH region according to any one of (1) to (7) above, and a VL region according to any one of (8) to (14) above. In some instances, the binding domain comprises a VH region according to any one of (1), (4) and (6) and a VL region according to any one of (8), (11) and (13). In other instances, the binding domain comprises a VH region according to any one of (2), (5) and (7) and a VL region according to any one of (9), (12), and (14).

### Anti-cMET Binding Domains

The binding domain that binds cMET (anti-cMET binding domain) typically comprises the CDRs of an antibody molecule which is capable of binding to cMET. In some instances, the binding domain that binds cMET additionally comprises the FRs of an antibody molecule which is capable of binding to cMET. That is, in some instances the binding domain that binds cMET comprises the VH region and the VL region of an antibody molecule which is capable of binding to cMET.

In some instances the binding domain that binds cMET comprises a VH region and a VL region which is, or which is derived from, the VH/VL region of a cMET-binding antibody clone described herein (i.e. anti-cMET antibody clone B09-GL).

In some instances the binding domain that binds cMET comprises the three HCDRs or three LCDRs, optionally the three VH CDRs and the three VL CDRs, of cMET-binding antibody clone B09-GL. The VH and VL domain sequences of antibodies B09-GL are described herein, and the three VH and three VL domain CDRs of said antibodies may thus be determined from said sequences.

In some instances, the binding domain that binds cMET comprises a VH region according to (15) below:
(15) a VH region comprising the following CDRs:
   HCDR1 having the amino acid sequence of SEQ ID NO: 24
   HCDR2 having the amino acid sequence of SEQ ID NO: 25
   HCDR3 having the amino acid sequence of SEQ ID NO: 26,
   or a variant thereof in which one or two or three amino acids in one or more of HCDR1, HCDR2, or HCDR3 are substituted with another amino acid.

In some instances, the binding domain that binds cMET comprises a VH region according to (15) above, wherein the VH region additionally comprises the FRs according to (16) below:
(16) HFR1 having the amino acid sequence of SEQ ID NO: 30
   HFR2 having the amino acid sequence of SEQ ID NO: 31
   HFR3 having the amino acid sequence of SEQ ID NO: 32
   HFR4 having the amino acid sequence of SEQ ID NO: 33,
   or a variant thereof in which one or two or three amino acids in one or more of HFR1, HFR2, HFR3, or HFR4 are substituted with another amino acid.

In some instances the binding domain that binds cMET comprises a VH according to (17) below:
(17) a VH region comprising the CDRs according to (15) and the FRs according to (16).

In some instances the binding domain that binds cMET comprises a VH region according to (18) below:
(18) a VH region comprising an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 38.

In some instances the binding domain that binds cMET comprises a VL region according to (19) below:
(19) a VL region comprising the following CDRs:
   LCDR1 having the amino acid sequence of SEQ ID NO: 27
   LCDR2 having the amino acid sequence of SEQ ID NO: 28
   LCDR3 having the amino acid sequence of SEQ ID NO: 29,
   or a variant thereof in which one or two or three amino acids in one or more of LCDR1, LCDR2, or LCDR3 are substituted with another amino acid.

In some instances, the binding domain that binds cMET comprises a VL region according to (19) above, wherein the VL region additionally comprises the FRs according to (20) below:
(20) LFR1 having the amino acid sequence of SEQ ID NO: 34
   LFR2 having the amino acid sequence of SEQ ID NO: 35
   LFR3 having the amino acid sequence of SEQ ID NO: 36
   LFR4 having the amino acid sequence of SEQ ID NO: 37,
   or a variant thereof in which one or two or three amino acids in one or more of LFR1, LFR2, LFR3, or LFR4 are substituted with another amino acid.

In some instances the binding domain that binds cMET comprises a VL region according to (21) below:
(21) a VL region comprising the CDRs according to (19) and the FRs according to (20).

In some instances the binding domain that binds cMET comprises a VL region according to (22) below:
(22) a VL region comprising an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 40.

In some instances the binding domain that binds cMET comprises a VH region according to any one of (15) to (18) above, and a VL region according to any one of (19) to (22) above.

### CDR Substitutions

In instances in accordance with the present disclosure, one or more amino acids (e.g. one, two or three) are substituted with another amino acid.

Naturally occurring residues may be divided into classes based on common side chain properties: 1) nonpolar, aliphatic: glycine (G), methionine (M), alanine (A), valine (V), leucine (L), isoleucine (I); 2) polar, uncharged: cysteine (C), serine (S), threonine (T), asparagine (N), glutamine (Q), proline (P); 3) acidic (negatively charged): aspartic acid (D), glutamic acid (E);
4) basic (positively charged): histidine (H), lysine (K), arginine (R);
5) aromatic: tryptophan (W), tyrosine (Y), phenylalanine (F).

The amino acid substitution may be a conservative amino acid substitution. Conservative amino acid substitutions may involve exchange of a member of one of these classes with another member of the same class. For example, a conservative amino acid substitution may be a substitution of the acidic amino acid glutamic acid (E) for the acidic amino acid aspartic acid (D).

In some instances, substitution(s) may be functionally conservative. That is, in some instances the substitution may not affect (or may not substantially affect) one or more functional properties (e.g. binding affinity) of the antigen-binding domain comprising the substitution as compared to the equivalent unsubstituted antigen-binding domain.

### Constant Region

In some instances the antibody molecule described herein comprises an immunoglobulin heavy chain constant (CH) region. In some instances the CH is, or is derived from, the heavy chain constant sequence of an IgG (e.g. IgG1, IgG2, IgG3, IgG4), IgA (e.g. IgAQ1, IgA2), IgD, IgE or IgM.

In some instances the CH region is human immunoglobulin G1 constant (IGHG1; UniProt: P01857-1, v1; SEQ ID NO: 42) or a fragment thereof.

In some instances, the CH region comprises an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 42, 43, 44, 45, 46, 63 or 64. In some instances, the CH region comprises an amino acid sequence having at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 63 or 64.

In some instances, the antibody molecule comprises a heavy chain that comprises or consists of a VH region as described herein and a CH region as described herein.

In some instances, the antibody molecule described herein comprises an immunoglobulin light chain constant (CL) region or a fragment thereof. In some instances, the CL region is, or is derived from a kappa CL region set forth in SEQ ID NO: 47 or SEQ ID NO: 48. In some instances, the CL region is, or is derived from a lambda CL region set forth in SEQ ID NO: 49 or SEQ ID NO: 65. In some instances, the antibody molecule comprises: a first CL region that is, or is derived from, a kappa CL region set forth in SEQ ID NO: 47 or 48; and a second CL region that is, or is derived from, a lambda CL region set forth in SEQ ID NO: 49 or 65.

In some instances, the antibody molecule described herein comprises:
a first heavy chain, wherein the first heavy chain comprises the VH region of the anti-EGFR binding domain, and a first heavy chain constant (CH) region or a fragment thereof;
a first light chain, wherein the first light chain comprises the VL region of the anti-EGFR binding domain, and a first light chain constant (CL) region or a fragment thereof;
a second heavy chain, wherein the second heavy chain comprises the VH region of the s anti-cMET binding domain, and a second heavy chain constant (CH) region or a fragment thereof; and
a second light chain, wherein the second light chain comprises the VL region of the anti-cMET binding domain, and a second light chain constant (CL) region or a fragment thereof.

The first and second CH region may be identical or different. In other words the first and second CH region may form a homodimer or heterodimer. For example, asymmetrical bispecific antibody molecules have different first and second CH regions, as described in more detail below. The first and second CL regions may be identical or different. In some instances, the first CL region is, or is derived from, a kappa CL region set forth in SEQ ID NO: 47 or 48; and a second CL region that is, or is derived from, a lambda CL region set forth in SEQ ID NO: 49 or 65.

It will be understood that when an antibody molecule comprises a first VH region and a first CH region, that these regions together form a first heavy chain of the antibody molecule, that is that the first VH and first CH regions are connected to each other. Similarly, a second VH region and a second CH region forms a second heavy chain of the antibody molecule; a first VL region and a first CL region form a first light chain of the antibody molecule; and a second VL region and a second CL region form a second light chain of the antibody molecule.

In some instances, the antibody molecule comprises a heavy chain having an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the B-09-GL heavy chain set forth in SEQ ID NO: 50, the QD6 heavy chain set forth in SEQ ID NO: 53, the RAA22 heavy chain set forth in SEQ ID NO: 56, the heavy chain set forth in SEQ ID NO: 59, or the heavy chain set forth in SEQ ID NO: 60.

In some instances, the antibody molecule comprises a first and second heavy chain, wherein
(i) the first heavy chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the B-09-GL heavy chain set forth in SEQ ID NO: 56; and
(ii) the second heavy chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the RAA22 heavy chain set forth in SEQ ID NO: 50.

In some instances, the antibody molecule comprises a first and second heavy chain, wherein
(i) the first heavy chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the heavy chain set forth in SEQ ID NO: 59; and
(ii) the second heavy chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the heavy chain set forth in SEQ ID NO: 60.

In some instances, the antibody molecule described herein comprises a light chain that comprises or consists of a VL region as described herein and a CL region as described herein.

In some instances, the antibody molecule described herein comprises a light chain having an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the B-09-GL light chain set forth in SEQ ID NO: 52, the QD6 light chain set forth in SEQ ID NO: 55, the RAA22 light chain set forth in SEQ ID NO: 58, the light chain set forth in SEQ ID NO: 61, or the light chain set forth in SEQ ID NO: 62.

In some instances, the antibody molecule described herein comprises a first and second light chain, wherein
(i) the first light chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the RAA22 light chain set forth in SEQ ID NO: 58; and
(ii) the second light chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the B-09-GL light chain set forth in SEQ ID NO: 52

In some instances, the antibody molecule described herein comprises a first and second light chain, wherein
(i) the first light chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the light chain set forth in SEQ ID NO: 61; and
(ii) the second light chain comprises an amino acid sequence which has at least 70% sequence identity, such as at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the light chain set forth in SEQ ID NO: 62.

The CH, CL, heavy chain and/or light chain of the antibody molecules described herein may comprise one or more modifications, for example to abrogate or reduce Fc effector functions, promote formation of a heterodimeric antibody molecule, to increase the efficacy of cognate heavy and light chain pairing, and/or to assist with conjugate formation as described in more detail below. A CH, CL, heavy chain and light chain that has been modified may be referred to as a modified CH, CL, heavy chain and light chain, respectively.

The antibody molecule may comprise a mutation in the CH region(s) of the heavy chain(s) to reduce or abrogate binding of the antibody molecule to one or more Fcγ receptors, such as FcγRI, FcγRIIa, FcγRIIb, FcγRIII and/or to complement. Such mutations abrogate or reduce Fc effector functions. Mutations for reduce or abrogate binding of antibody molecule to one or more Fcγ receptors and complement are known and include the “triple mutation” or “TM” of L234F/L235E/P331S described for example in Organesyan, 2008. Other mutations that are known to modulate antibody effector function are described for example in Wang, 2018.

Thus, in some instances the first and/or second heavy chain comprise phenylalanine (F) at position 234, glutamic acid (E) at position 235, and serine (S) at position 331, wherein the numbering is as per the EU index. For example, one or both of the first and second heavy chains may comprise a CH region having an amino acid sequence having at least 70%, at least 80%, at least 90%, at least 95% sequence identity to the sequence set forth in SEQ ID NO: 42 and comprise a phenylalanine (F) at position 234, glutamic acid (E) at position 235, and serine (S) at position 331, wherein the numbering is as per the EU index. As demonstrated in the examples (e.g. Example 12), including the TM in the heavy chain was demonstrated to improve pharmacokinetic properties of the exemplified antibody molecules and ADCs.

Examples of CH regions comprising the triple mutation are SEQ ID NOs: 63 and 64. Thus, in some instances, one of the first and second heavy chains comprises a CH region having an amino acid sequence having at least 70%, at least 80%, at least 90%, at least 95%, or 100% sequence identity to the sequence set forth in SEQ ID NO: 63 and the other heavy chain comprises a CH region having an amino acid sequence having at least 70%, at least 80%, at least 90%, at least 95%, or 100% sequence identity to the sequence set forth in SEQ ID NO: 64, wherein one or both of the CH regions comprise a phenylalanine at position 234, glutamic acid at position 235, and serine at position 331, wherein the numbering is as per the EU index.

Examples of heavy chains comprising a CH region containing the triple mutation are SEQ ID NOs: 59 and 60. Thus, in some instances, one of the first and second heavy chains has an amino acid sequence having at least 70%, at least 80%, at least 90%, at least 95%, or 100% sequence identity to the sequence set forth in SEQ ID NO: 59 and the other heavy chain has an amino acid sequence having at least 70%, at least 80%, at least 90%, at least 95%, or 100% sequence identity to the sequence set forth in SEQ ID NO: 60, wherein one or both of the have chains comprise a phenylalanine at position 234, glutamic acid at position 235, and serine at position 331, wherein the numbering is as per the EU index.

The VL and CL region, and the VH region and CH1 region of an antibody molecule together constitute the Fab region. The remainder of the antibody molecule constitute the Fc region.

Unless otherwise specified, amino acid residue positions in the constant domain, including the position of amino acid sequences, substitutions, deletions and insertions as described herein, are numbered according to EU numbering (Edelman, 2007).

### Bispecific Formats

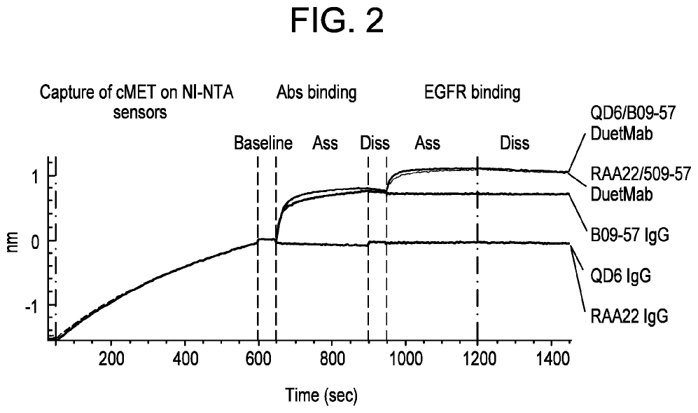

Bispecific antibody molecules may be provided in any suitable format. Suitable formats for a bispecific antibody molecule described herein, and methods for producing the same, are described in Kontermann, MAbs 2012, 4(2):182-197 and Kontermann and Brinkmann 2015, 20(7): 838-847, both of which are herein incorporated by reference in their entirety. See in particular FIG. 2 of Kontermann MAbs 2012, 4(2): 182-19.

Bispecific antibody molecules can also be generated from existing antibodies by chemical conjugation. For example, two IgG molecules or two Fab′ fragments can be coupled using homo- or hetero-bifunctional coupling reagents, e.g. as described in Graziano and Guptill, Methods Mol Biol. 2004; 283:71-85.

In some instances, the bispecific antibody molecules may be an immunoglobulin G-like (IgG-like) bispecific antibody molecule. IgG-like bispecific antibody molecule may comprise an Fv region, Fab region or sVD specific for one antigen, an Fv/Fab/sVD specific for another antigen, and an Fc region. IgG-like bispecific antibody molecules may be either symmetrical or asymmetrical. In one instance, the bispecific antibody molecule is asymmetrical.

Symmetrical IgG-like bispecific antibody molecules generally contain an antigen-binding domain that is fused to the N- or C-terminus of the heavy of light chain of an IgG molecule, e.g. in the form of a scFv fragment or a variable single domain. A characteristic property of these symmetrical IgG-like bispecific antibody molecules is that they contain a two identical heavy chains. Furthermore, symmetrical IgG-like bispecific antibody molecules are typically bivalent for each epitope. Valency as used herein refers to the number of antigen-binding regions in the antibody molecule that are able to bind a single epitope. A monoclonal monospecific IgG antibody molecule is bivalent for a single epitope—it contains two antigen-binding domains, each of which are able to bind an epitope on a single target molecule. A symmetrical IgG-like bispecific antibody molecule is bivalent for each epitope—it typically contains four antigen-binding domains, two of which are able to bind a first epitope on a target molecule and two of which are able to bind to a second epitope on a target molecule.

Examples of symmetrical IgG-like bispecific antibody molecules include DVD-IgG, IgG-scFv, scFv-IgG, scFv4-Ig, IgG-scFab, scFab-IgG, IgG-sVD, sVD-IgG, 2 in 1-IgG, mAb², tandemab common LC. These can be formed by methods known in the art, for example chemical crosslinking, somatic hybridisation or the redox method.

Asymmetrical IgG-like bispecific antibody molecules, in contrast, are typically monovalent for each target. As described in, for example, Klein, 2012, the concept of monovalent bispecific IgG is thought to have a unique therapeutic niche in that they (i) do not cause receptor homodimerization, (ii) potentially have reduced toxicity on non-target tissues due to loss of avidity for each antigen, and (iii) have better selectivity when both antigens are either selectively restricted or abundantly expressed on target cells. Thus, in some instances, the antibody molecule is an asymmetrical IgG-like bispecific antibody molecule.

Asymmetrical IgG-like bispecific antibody molecules involve heterodimerization of two district heavy chain and correct pairing of the cognate light chain and heavy chain. Heterodimerization of the heavy chains can be addressed by several techniques, such as knobs-into-holes, electrostatic steering of CH3, CH3 strand exchanged engineered domains and leucine zippers. The pairing of the correct light and heavy chain can be ensured by using one of these heavy chain heterodimerization techniques along with the use of a common light chain, domain cross-over between CH1 and CL, coupling of the heavy and light chains with a linker, in vitro assembly of heavy chain-light chain dimers from two separate monoclonals, interface engineering of an entire Fab domain, or disulfide engineering of the CH1/CL interface.

Examples of asymmetrical IgG-like bispecific antibody molecules include DuetMab, kih IgG, kih IgG common LC, CrossMab, kih IgG-scFab, mAb-Fv, charge pairs and SEED-body.

In some instances, the antibody molecules comprise one or more modifications in one or more of the CH1, CH2 and CH3 domains that promotes formation of a heterodimeric antibody molecule. For example, the DuetMab antibody molecule described above may additionally comprise one or more modifications in one or more of the CH1, CH2 and CH3 domains that promotes formation of a heterodimeric antibody molecule. This may involve a Knobs into Holes (KiH) strategy based on single amino acid substitutions in the CH3 domains that promote heavy chain heterodimerization is described in Ridgway, 1996. The knob variant heavy chain CH3 has a small amino acid has been replaced with a larger one, and the hole variant has a large amino acid has replaced with a smaller one. Additional modifications may also introduced to stabilise the association between the heavy chains.

CH3 modifications to enhance heterodimerization include, for example, Y407V/T366S/L368A on one heavy chain and T366W on the other heavy chain; and S354C/T366W on one heavy chain and Y349C/Y407V/T366S/L368A on the other heavy chain, wherein the numbering of the constant region is as per the EU index.

Other examples of CH3 modification to enhance heterodimerization are described in, e.g. Table 1 of Brinkmann and Kontermann, 2017 MABS 9(2), 182-212, which is herein specifically incorporated by reference.

In some instances, the antibody molecule comprises a first and second heavy chain that form a heterodimer, wherein one of the first and second heavy chains comprises a cysteine (C) residue at position 354 and a tryptophan (W) residue at position 366 and the other heavy chain comprises a cysteine (C) residue at position 349, a valine (V) residue at position 407, a serine (S) at position 366 and an alanine (A) at position 368, wherein the numbering of the constant region is as per the EU index. For example, the one of the first and second heavy chains may have the sequence set forth in SEQ ID NO: 42 and further comprise a cysteine (C) residue at position 354 and a tryptophan (W) residue at position 366, and the other heavy chain have the sequence set forth in SEQ ID NO: 42 and further comprise a cysteine (C) residue at position 349, a valine (V) residue at position 407, a serine (S) at position 366 and an alanine (A) at position 368, wherein the numbering of the constant region is as per the EU index.

In some instances, the antibody molecule comprises:
(i) a first heavy chain comprising a first modified CH3 region, wherein the first modified CH3 region comprises a cysteine (C) residue at position 354 and a tryptophan (W) residue at position 366; and
(ii) a second heavy chain comprising a second modified CH3 region, wherein the second modified CH3 region comprises a cysteine (C) residue at position 349, a valine (V) residue at position 407, a serine (S) at position 366 and an alanine (A) at position 368, wherein the numbering of the constant region is as per the EU index.

A particular exemplified format of asymmetrical IgG-like bispecific antibody molecules is referred to as “DuetMab”. DuetMab antibody molecules uses KIH technology for heterodimerization of 2 distinct heavy chains and increases the efficacy of cognate heavy and light chain pairing by replacing the native disulphide bond in one of the CH1-CL interfaces with an engineered disulphide bond. Disclosure related to DuetMab can found e.g., in U.S. Pat. No. 9,527,927 and Mazor, 2015, which are herein incorporated by reference in their entirety.

In some instances, the antibody molecule comprises:
(a) a modified CH region, wherein the modified heavy chain comprises a substitution of a native non-cysteine amino acid to a cysteine amino acid; and
(b) a modified corresponding CL region, wherein the modified CL comprises a substitution of a native non-cysteine amino acid to a cysteine amino acid,
wherein either:
(i) the first heavy chain comprises the modified CH region and the first light chain comprises the modified corresponding CL region; or
(ii) the second heavy chain comprises the modified CH region and the second light chain comprises the modified corresponding CL region.

In some instances, the substituted cysteine of the modified CH region, resulting from the substitution of the native non-cysteine amino acid to the cysteine amino acid, and the substituted cysteine of the modified corresponding CL region, resulting from the substitution of the native non-cysteine amino acid to the cysteine amino acid, can form a disulphide bond.

In some instances, the modified CH region comprises a substitution of a native non-cysteine amino acid to a cysteine amino acid at position 126; and the modified corresponding CL region comprises a substitution of a native non-cysteine amino acid to a cysteine at position 121, wherein the numbering of the constant region is as per the EU index.

In some instances, the modified CH region comprises a substitution of a native non-cysteine amino acid to a cysteine amino acid at position 126 and a substitution of a native cysteine amino acid to a non-cysteine amino acid at position 219, for example to a valine; and the modified corresponding CL region comprises a substitution of a native non-cysteine amino acid to a cysteine at position 121 and a substitution of a native cysteine amino acid to a non-cysteine amino acid at position 214, for example to a valine, where the numbering of the constant region is as per the EU index.

In some instances, the antibody molecule comprises a second CH region and a second corresponding light chain, wherein the second CH region and second corresponding CL do not comprise a substitution of a native non-cysteine amino acid to a cysteine amino acid and do not comprise a substitution of a native cysteine to a non-cysteine amino acid.

### Conjugates

The antibody molecule may be conjugated to a drug. In this case, the antibody molecule may be referred to as a “conjugate” or an “antibody drug conjugate”. Such conjugates find application in the treatment and/or diagnosis of diseases as described herein. As used herein, the drug may be referred to as a “payload” or “warhead”.

In some instances, the drug comprises a cytotoxin, a radioisotope, an immunomodulator, a cytokine, a lymphokine, a chemokine, a growth factor, a tumor necrosis factor, a hormone, a hormone antagonist, an enzyme, an oligonucleotide, a DNA, an RNA, an siRNA, an RNAi, a microRNA, a photoactive therapeutic agent, an anti-angiogenic agent, a pro-apoptotic agent, a peptide, a lipid, a carbohydrate, a chelating agent, or combinations thereof.

A cytotoxin is a compound that is able to include death of the cell that is being targeted. Typically, in the context of antibody drug conjugates, a cytotoxin is delivered to a cell targeted by the antibody molecule, where it is released into the cell and induces cell death. The use of cytotoxins in antibody drug conjugates is described, for example, in Chalouni and Doll 2018 J Exp Clin Cancer Res. 37(1):20.

In some instances, cytotoxin is a tubulysin, an auristatin, a maytansinoid, a topoisomerase inhibitor or a pyrrolobenzodiazepine (PBD).

In particular instances, the cytotoxin is or comprises a tubulysin. Tubulysins are a class of cytostatic tetrapeptides which contain isoleucine and three other complex unnatural amino acids Mep (R-N-Mepipecolic acid), Tuv (tubuvaline) and Tut (tubulyrosine) or Tup (tubuphenylalanine). Tubulysins are extremely potent cytotoxic molecules and are potent against multidrug resistant cell lines (Domling, 2005). These compounds show high cytotoxicity tested against a panel of cancer cell lines with IC50 values in the low picomolar range; thus, they are of interest as anticancer therapeutics. See, e.g., WO2012019123. Tubulysin conjugates are disclosed, e.g., in U.S. Pat. No. 7,776,814. In some instances, the tubulysin is tubulysin A having the following chemical structure:

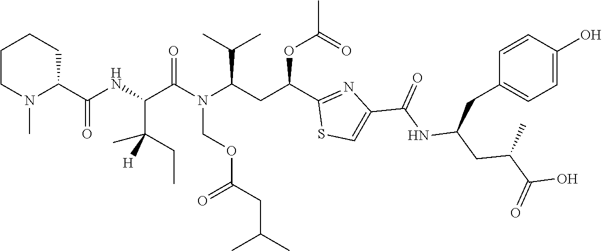 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C]

In some instances, the tubulysin is tubulysin 1508, also referred to as “AZ1508” and described in more detail in WO 2015157594. Tubulysin 1508 has the following chemical structure:

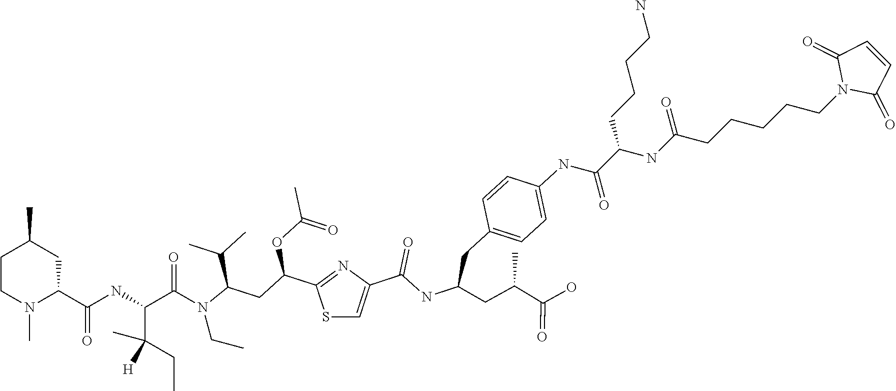 [SMILES: CC[C@H](C)[C@H](NC(=O)[C@H]1C[C@H](C)CCN1C)C(=O)N(CC)[C@H](C[C@@H](OC(C)=O)c1nc(C(=O)N[C@@H](Cc2ccc(NC(=O)[C@H](CCCCN)NC(=O)CCCCCN3C(=O)C=CC3=O)cc2)C[C@H](C)C(=O)O)cs1)C(C)C]

In some instances, the cytotoxin is or comprises a topoisomerase inhibitor. The term ‘topoisomerase inhibitor’ as used herein refers to a cytotoxic agent that inhibits the activity of one or more of the topoisomerase enzymes (topoisomerase I and II), which are enzymes that play an important role in DNA replication and transcription by regulating DNA supercoiling. Antibody drug conjugates comprising a topoisomerase inhibitor as a cytotoxin are therefore expected to interfere with normal processes involving DNA, therefore leading to cell death. Conjugates containing topoisomerase inhibitors have been demonstrated to be effective against a variety of tumor containing cell lines as well as having anticancer activity in clinical trials. See for example Ogitani, 2016a; Ogitani, 2016b; Cardillo, 2015; and Bardia, 2017.

In some instances, the antibody molecule is conjugated to a topoisomerase I inhibitor. Representative examples of topoisomerase I inhibitors include, but are not limited to, camptothecins and its analogues topotecan, irinotecan, belotecan, exatecan, lurotecan and sinotecan. Representative examples of topoisomerase II inhibitors include, but are not limited to, amsacrine, daunorubicin, doxorubicin, epipodophyllotoxins, ellipticines, epirubicin, etoposide, razoxane, and teniposide.

An example of the camptothecin chemical structure is as follows:

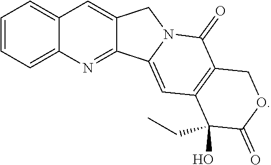 [SMILES: CC[C@@]1(O)C(=O)OCc2c1cc1n(c2=O)Cc2cc3ccccc3nc2-1]

A general example of a suitable topoisomerase I inhibitor is represented by the following compound:

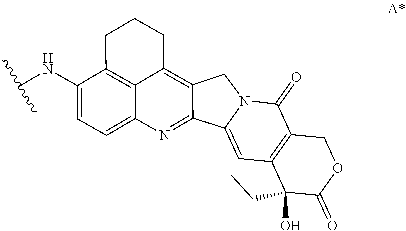 [SMILES: *Nc1ccc2nc3c(c4c2c1CCC4)Cn1c-3cc2c(c1=O)COC(=O)[C@]2(O)CC]

Said compound is denoted as A*.

In some instances, the compound (e.g. A*) is provided with a linker for connecting to an antibody molecule described herein (which may be referred to as a “Ligand Unit” or alternatively a “Cell Binding Agent” (CBA)). Suitably, the linker is attached (e.g. conjugated) in a cleavable manner to an amino residue, for example, an amino acid of an antibody molecule described herein.

The design and selection of linkers to be used in conjugates is known in the art and is described for example in Beck, 2017. The linker used herein may be any of the linkers described in Beck, 2017.

More particularly, an example of a suitable topoisomerase I inhibitor is represented by the following compound, with the formula “I”:

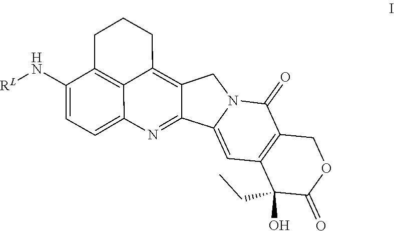

and salts and solvates thereof, wherein R^{L} is a linker for connection to an antibody molecule described herein, wherein said linker is optionally selected from:

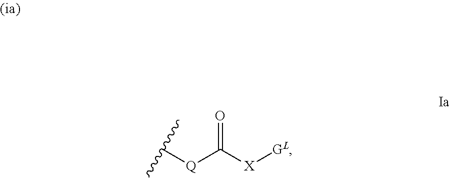

wherein
Q is:

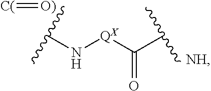

where Q^{X} is such that Q is an amino-acid residue, a dipeptide residue, a tripeptide residue or a tetrapeptide residue;
X is:

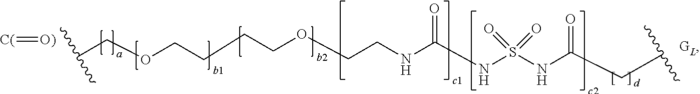

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least cl or c2=0 (i.e. only one of c1 and c2 may not be 0);
G^{L} is a linker for connecting to an antibody or antigen binding fragment thereof described herein (e.g. the Ligand Unit or Cell Binding Agent); or

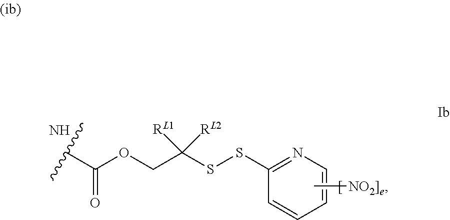

where R^{L1} and R^{L2} are independently selected from H and methyl, or together with the carbon atom to which they are bound form a cyclopropylene or cyclobutylene group; and
e is 0 or 1.

In the formula

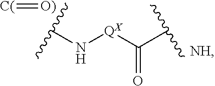

the superscripted labels C(═O) and NH indicate the group to which the atoms are bound. For example, the NH group is shown as being bound to a carbonyl (which is not part of the moiety illustrated), and the carbonyl is shown as being bound to a NH group (which is not part of the moiety illustrated).

It will be understood by the person skilled in the art that more than one of said agent(s) (e.g. topoisomerase I inhibitor) may be conjugated to the antibody molecule.

For example, a conjugate (e.g. antibody-drug conjugate) of the disclosure may be of the general formula IV:

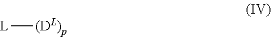

or a pharmaceutically acceptable salt or solvate thereof, wherein L is an antibody molecule described herein (e.g. the Ligand Unit or CBA), D^{L} is drug having a linker (e.g. a Drug Linker Unit), and p is a integer of from 1 to 20.

In some instances, D^{L} is a topoisomerase I inhibitor having a linker that is of formula III:

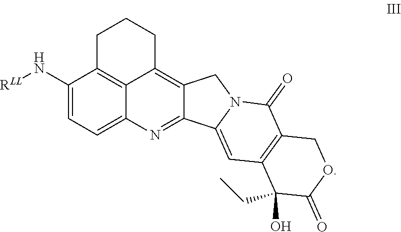

R^{LL} is a linker connected to an antibody molecule described herein (e.g. the Ligand Unit), wherein the linker is optionally selected from

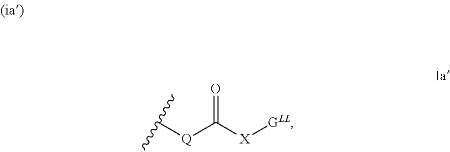

where Q and X are as defined above and G^{LL} is a linker connected to an antibody molecule described herein (e.g. the Ligand Unit or CBA); and

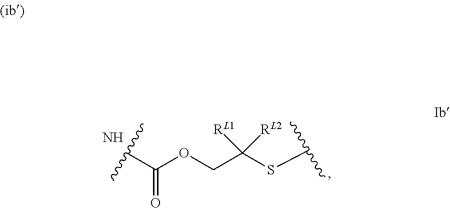

where R^{L1} and R^{L2} are as defined above.

The drug loading is represented by p, the number of topoisomerase I inhibitor(s) (e.g. Drug units) per antibody molecule (e.g. Ligand Unit). Drug loading may range from 1 to 20 Drug units (D) per Ligand unit. For compositions, p represents the average drug loading of the conjugates in the composition, and p ranges from 1 to 20. In some instances, where the drug is a topoisomerase inhibitor, the p range is selected from 2 to 8, optionally 4 to 8, such as 5 to 7, or 5.5 to 6.5. As described in the examples, an ADC comprising topoisomerase I inhibitor SG3932 was produced with an average DAR of 6+/−6%.

Accordingly, the disclosure embraces a conjugate comprising an antibody molecule described herein (e.g. the Ligand Unit or CBA) covalently linked to at least one topoisomerase I inhibitor (e.g. Drug unit, such as A* illustrated above). Said inhibitor is optionally linked to the antibody molecule by a linker (e.g. Linker unit), such as a linker described above as R^{L} and/or R^{LL}. In other words, the disclosure embraces an antibody molecule described herein (e.g. the Ligand Unit or CBA) with one or more topoisomerase I inhibitors attached, optionally via a linker (e.g. Drug-Linker units). The antibody molecule (representing a Ligand unit or CBA), described more fully above, is a targeting agent that binds to a target moiety. More particularly, this antibody molecule can, for example, specifically binds to a EGFR and cMET on a target cell, to which the Drug unit is thus delivered. Accordingly, the present disclosure also provides methods for the treatment of, for example, various cancers and other disorders with an ADC (e.g. cancers/disorders which are associated with the presence of cells, such as cancerous cells, which express EGFR and cMET). Such methods are described in more detail below Q^{X}

In one instance, Q is an amino acid residue. The amino acid may be a natural amino acid or a non-natural amino acid. For example, Q may be selected from: Phe, Lys, Val, Ala, Cit, Leu, Ile, Arg, and Trp, where Cit is citrulline.

In one instance, Q comprises a dipeptide residue. The amino acids in the dipeptide may be any combination of natural amino acids and non-natural amino acids. In some instances, the dipeptide comprises natural amino acids. Where the linker is a cathepsin labile linker, the dipeptide is the site of action for cathepsin-mediated cleavage. The dipeptide then is a recognition site for cathepsin.

In one instance, Q is selected from:

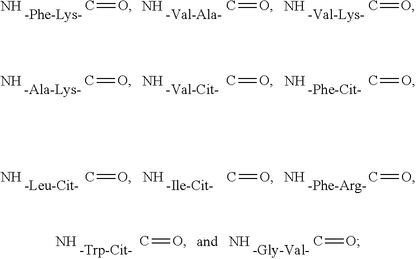

where Cit is citrulline.

In one instance, Q is selected from:

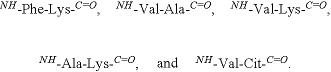

In one instance, Q is selected from NH-Phe-Lys-C═O-Val-Cit-C═O or NH-Val-Ala-C═O.

Other suitable dipeptide combinations include:

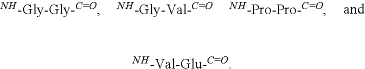

Other dipeptide combinations may be used, including those described by Dubowchik et al., Bioconjugate Chemistry, 2002, 13, 855-869, which is incorporated herein by reference.

In some instances, Q is a tripeptide residue. The amino acids in the tripeptide may be any combination of natural amino acids and non-natural amino acids. In some instances, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:

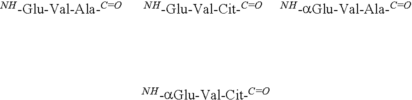

In some instances, Q is a tetrapeptide residue. The amino acids in the tetrapeptide may be any combination of natural amino acids and non-natural amino acids. In some instances, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:

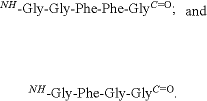

In some instances, the tetrapeptide is:

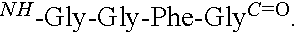

In the above representations of peptide residues, NH— represents the N-terminus, and —C═O represents the C-terminus of the residue. The C-terminus binds to the NH of A*.

Glu represents the residue of glutamic acid, i.e.:

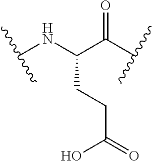 [SMILES: *N[C@@H](CCC(=O)O)C(*)=O]

αGlu represents the residue of glutamic acid when bound via the α-chain, i.e.:

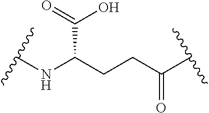 [SMILES: *N[C@@H](CCC(*)=O)C(=O)O]

In one instance, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

### G^{L}

G^{L} may be selected from:

| | |
|---|---|
| (G^{L1-1}) | 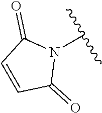 [SMILES: *N1C(=O)C=CC1=O] |
| (G^{L1-2}) | 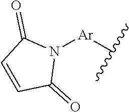 |
| (G^{L2}) | 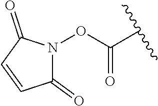 [SMILES: *C(=O)ON1C(=O)C=CC1=O] |
| (G^{L3-1}) | 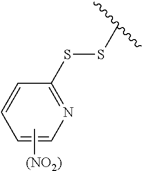 |
| | where the NO₂ group is optional |
| (G^{L3-2}) | 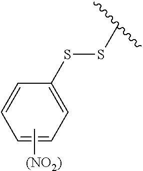 |
| | where the NO₂ group is optional |
| (G^{L3-3}) | 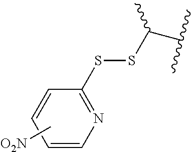 |
| | where the NO₂ group is optional |
| (G^{L3-4}) | 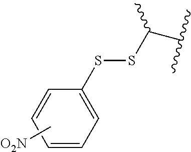 |
| | where the NO₂ group is optional |
| (G^{L4}) | 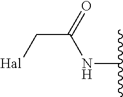 |
| | Where Hal = I, Br, Cl |
| (G^{L5}) | 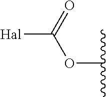 |
| (G^{L6}) | 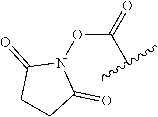 [SMILES: *C(=O)ON1C(=O)CCC1=O] |
| (G^{L7}) | 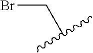 [SMILES: *CBr] |
| (G^{L8}) | 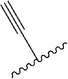 [SMILES: *C#C] |
| (G^{L9}) | 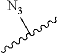 [SMILES: *N=[N+]=[N-]] |
| (G^{L10}) | 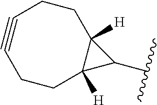 [SMILES: *C1[C@H]2CCC#CCC[C@@H]12] |
| (G^{L11}) | 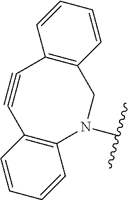 [SMILES: *N1Cc2ccccc2C#Cc2ccccc21] |
| (G^{L12}) | 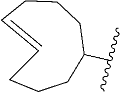 [SMILES: *C1CCC/C=C/CCC1] |
| (G^{L13}) | 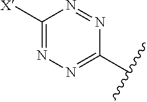 |
| (G^{L14}) | 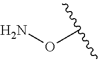 [SMILES: *ON] |

where Ar represents a C₅₋₆ arylene group, e.g. phenylene, and X′ represents C1-4 alkyl.

In some instances, G^{L} is selected from G^{L1-1} and G^{L1-2}. In some of these instances, G^{L} is G^{L1-1}.

### G^{LL}

G^{LL} may be selected from:

| | |
|---|---|
| (G^{LL1-1}) | 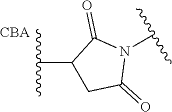 [SMILES: *C1CC(=O)N(*)C1=O] |
| (G^{LL1-2}) | 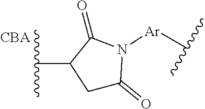 |
| (G^{LL2}) | 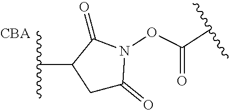 [SMILES: *C(=O)ON1C(=O)CC(*)C1=O] |
| (G^{LL3-1}) | 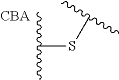 [SMILES: *S*] |
| (G^{LL3-2}) | 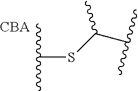 [SMILES: *SC(*)C] |
| (G^{LL-4}) | 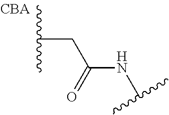 [SMILES: *CC(=O)N*] |
| (G^{LL5}) | 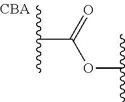 [SMILES: *OC(*)=O] |
| (G^{LL6}) | 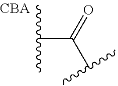 [SMILES: *C(*)=O] |
| (G^{LL7}) | 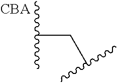 [SMILES: *C*] |
| (G^{LL8-1}) | 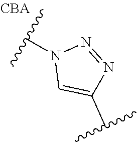 [SMILES: *c1cn(*)nn1] |
| (G^{LL8-2}) | 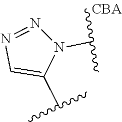 [SMILES: *c1cnnn1*] |
| (G^{LL9-1}) | 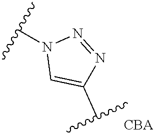 [SMILES: *c1cn(*)nn1] |
| (G^{LL9-2}) | 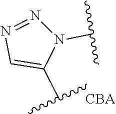 [SMILES: *c1cnnn1*] |
| (G^{LL10}) | 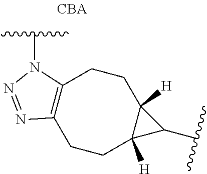 [SMILES: *C1[C@H]2CCc3c(nnn3*)CC[C@@H]12] |
| (G^{LL11}) | 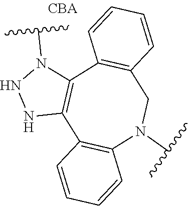 [SMILES: *N1NNC2=C1c1ccccc1CN(*)c1ccccc12] |
| (G^{LL12}) | 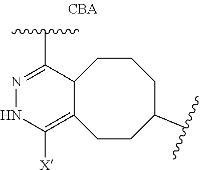 |
| (G^{LL13}) | 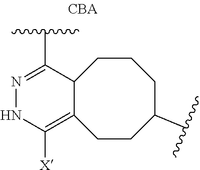 |
| (G^{LL14}) | 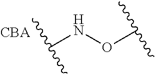 [SMILES: *NO*] |

where Ar represents a C₅₋₆ arylene group, e.g. phenylene and X′ represents C₁₋₄ alkyl. CBA represents the Cell Binding Agent or Ligand Unit.

In some instances, G^{LL} is selected from G^{LL1-1} and G^{LL1-2}. In some of these instances, G^{LL} is G^{LL-1}.

### X

X is optionally:

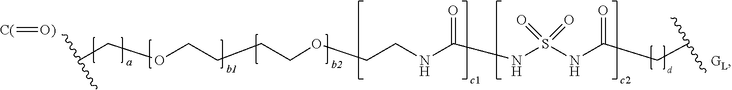

where a=0 to 5, b1=0 to 16, b2=0 to 16, c=0 or 1, d=0 to 5, wherein at least b1 or b2=0 and at least c1 or c2=0.

a may be 0, 1, 2, 3, 4 or 5. In some instances, a is 0 to 3. In some of these instances, a is 0 or 1. In further instances, a is 0.

b1 may be 0, 1, 2, 3, 4, 5, 6, 7, 8, 9, 10, 11, 12, 13, 14, 15 or 16. In some instances, b1 is 0 to 12. In some of these instances, b1 is 0 to 8, and may be 0, 2, 3, 4, 5 or 8.

b2 may be 0, 1, 2, 3, 4, 5, 6, 7, 8, 9, 10, 11, 12, 13, 14, 15 or 16. In some instances, b2 is 0 to 12. In some of these instances, b2 is 0 to 8, and may be 0, 2, 3, 4, 5 or 8. In some instances, only one of b1 and b2 may not be 0.

c1 may be 0 or 1. c2 may be 0 or 1. In some instances, only one of c1 and c2 may not be 0.

d may be 0, 1, 2, 3, 4 or 5. In some instances, d is 0 to 3. In some of these instances, d is 1 or 2. In further instances, d is 2. In further instances, d is 5.

In some instances of X, a is 0, b1 is 0, c1 is 1, c2 is 0 and d is 2, and b2 may be from 0 to 8. In some of these instances, b2 is 0, 2, 3, 4, 5 or 8. In some instances of X, a is 1, b2 is 0, c1 is 0, c2 is 0 and d is 0, and b1 may be from 0 to 8. In some of these instances, b1 is 0, 2, 3, 4, 5 or 8. In some instances of X, a is 0, b1 is 0, c1 is 0, c2 is 0 and d is 1, and b2 may be from 0 to 8. In some of these instances, b2 is 0, 2, 3, 4, 5 or 8. In some instances of X, bi is 0, b2 is 0, c1 is 0, c2 is 0 and one of a and d is 0. The other of a and d is from 1 to 5. In some of these instances, the other of a and d is 1. In other of these instances, the other of a and d is 5. In some instances of X, a is 1, b2 is 0, c1 is 0, c2 is 1, d is 2, and b1 may be from 0 to 8. In some of these instances, b2 is 0, 2, 3, 4, 5 or 8.

In some instances, R^{L} is of formula Ib. In some instances, R^{LL} is formula Ib′.

R^{L1} and R^{L2} may be independently selected from H and methyl, or together with the carbon atom to which they are bound form a cyclopropylene or cyclobutylene group.

In some instances, both R^{L1} and R^{L2} are H. In some instances, R^{L1} is H and R^{L2} is methyl. In some instances, both R^{L1} and R^{L2} are methyl.

In some instances, R^{L1} and R^{L2} together with the carbon atom to which they are bound form a cyclopropylene group. In some instances, R^{L1} and R^{L2} together with the carbon atom to which they are bound form a cyclobutylene group.

In the group Ib, in some instances, e is 0. In other instances, e is 1 and the nitro group may be in any available position of the ring. In some of these instances, it is in the ortho position. In others of these instances, it is in the para position.

In some instances where compounds described herein are provided in a single enantiomer or in an enantiomerically enriched form, the enantiomerically enriched form has an enantiomeric ratio greater than 60:40, 70:30; 80:20 or 90:10. In further instances, the enantiomeric ratio is greater than 95:5, 97:3 or 99:1.

In some instances, R^{L} is selected from:

| | |
|---|---|
| (i) | 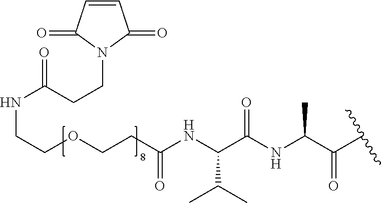 |
| (ii) | 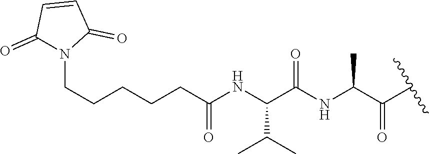 [SMILES: *C(=O)[C@H](C)NC(=O)[C@@H](NC(=O)CCCCCN1C(=O)C=CC1=O)C(C)C] |
| (iii) | 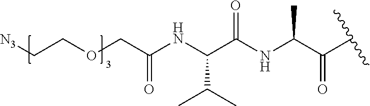 |
| (iv) | 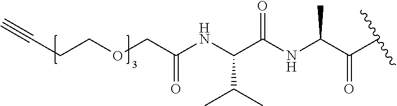 |
| (v) | 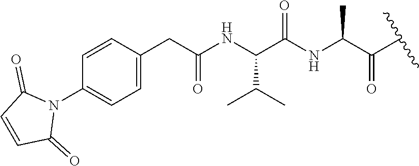 [SMILES: *C(=O)[C@H](C)NC(=O)[C@@H](NC(=O)Cc1ccc(N2C(=O)C=CC2=O)cc1)C(C)C] |
| (vi) | 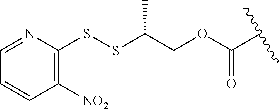 [SMILES: *C(=O)OC[C@@H](C)SSc1ncccc1[N+](=O)[O-]] |
| (vii) | 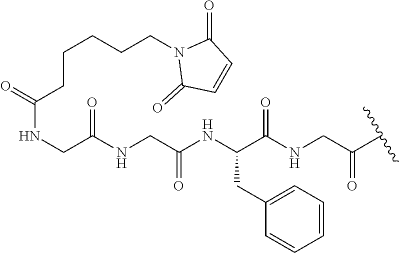 [SMILES: *C(=O)CNC(=O)[C@H](Cc1ccccc1)NC(=O)CNC(=O)CNC(=O)CCCCCN1C(=O)C=CC1=O] |
| (viii) | 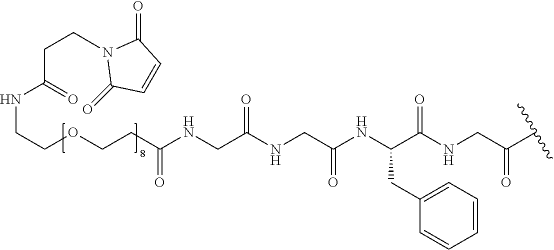 |
| (ix) | 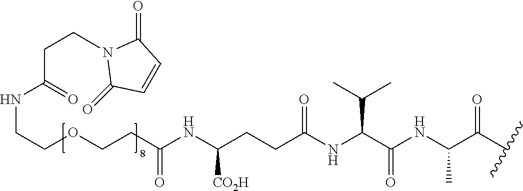 |

In some instances, R^{LL} is a group derived from the R^{L} groups above.

In some instances, the compound of formula I is of the formula I^{P}:

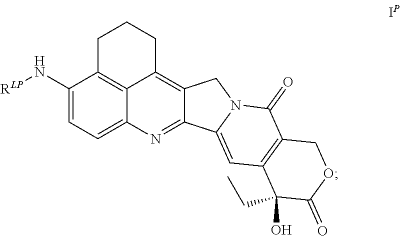

and salts and solvates thereof, wherein R^{LP} is a linker for connection to an antibody or antigen binding fragment thereof described herein, wherein said linker is selected from:

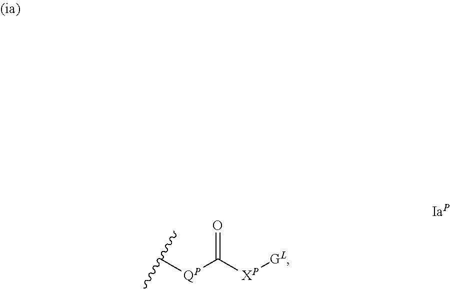

wherein
Q^{P} is:

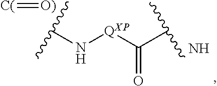

where Q^{XP} is such that Q^{P} is an amino-acid residue, a dipeptide residue or a tripeptide residue;
X^{P} is:

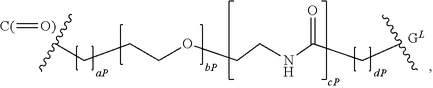

where aP=0 to 5, bP=0 to 16, cP=0 or 1, dP=0 to 5;
G^{L} is a linker for connecting to an antibody or antigen binding fragment thereof described herein (e.g. Ligand Unit);

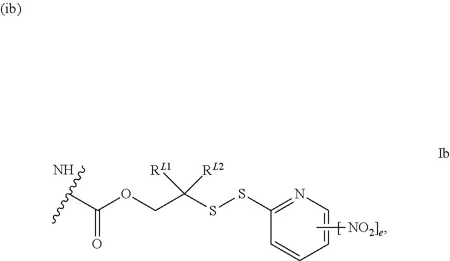

where RY and R^{L2} are independently selected from H and methyl, or together with the carbon atom to which they are bound form a cyclopropylene or cyclobutylene group; and
e is 0 or 1.

aP may be 0, 1, 2, 3, 4 or 5. In some instances, aP is 0 to 3. In some of these instances, aP is 0 or 1. In further instances, aP is 0.

bP may be 0, 1, 2, 3, 4, 5, 6, 7, 8, 9, 10, 11, 12, 13, 14, 15 or 16. In some instances, b is 0 to 12. In some of these instances, bP is 0 to 8, and may be 0, 2, 4 or 8.

cP may be 0 or 1.

dP may be 0, 1, 2, 3, 4 or 5. In some instances, dP is 0 to 3. In some of these instances, dP is 1 or 2. In further instances, dP is 2.

In some instances of XP, aP is 0, cP is 1 and dP is 2, and bP may be from 0 to 8. In some of these instances, bP is 0, 4 or 8.

The instances for Q^{X} above for compounds of Formula I may apply to Q^{XP} (for example, where appropriate).

The instances for G^{L}, R^{L1}, R^{L2} and e above for compounds of Formula I may apply to compounds of Formula I^{P}.

In some instances, the conjugate of formula IV is of the formula IV^{P}:

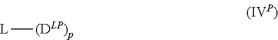

or a pharmaceutically acceptable salt or solvate thereof, wherein L is an antibody or antigen binding fragment thereof described herein (e.g. Ligand Unit), D^{LP} is a topoisomerase I inhibitor (e.g. Drug Linker unit) that is of formula IIIP:

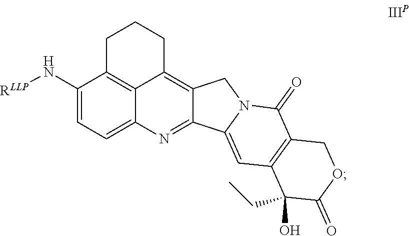

R^{LLP} is a linker connected to the antibody or antigen binding fragment thereof (e.g. Ligand unit), wherein said linker is selected from

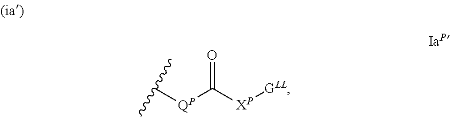

where Q^{P} and X^{P} are as defined above and G^{LL} is a linker connected to an antibody or antigen binding fragment thereof described herein (e.g. Ligand Unit); and

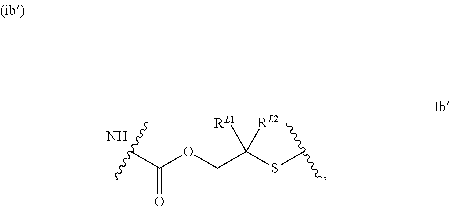

where R^{L1} and R^{L2} are as defined above; and
p is an integer of from 1 to 20.

In some instances, the compound of formula I is of the formula I^{P2}:

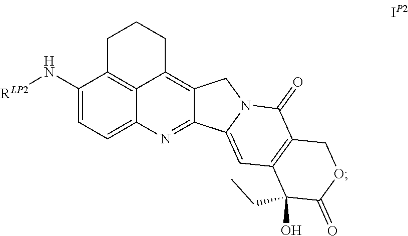

and salts and solvates thereof, wherein R^{LP2} is a linker for connection to an antibody or antigen binding fragment thereof described herein, wherein said linker is selected from:

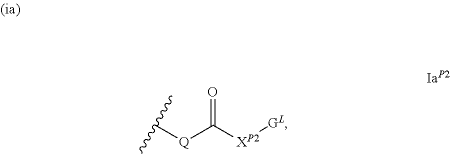

wherein
Q is:

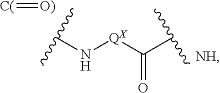

where Q^{X} is such that Q is an amino-acid residue, a dipeptide residue, a tripeptide residue or a tetrapeptide residue;
X^{P2} is:

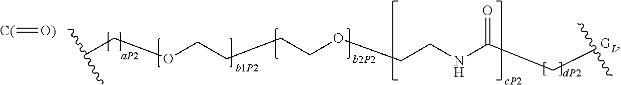

where aP2=0 to 5, b1P2=0 to 16, b2P2=0 to 16, cP2=0 or 1, dP2=0 to 5, wherein at least b1P2 or b2P2=0 (i.e. only one of b1 and b2 may not be 0);
G^{L} is a linker for connecting to an antibody or antigen binding fragment thereof described herein (e.g. Ligand Unit);

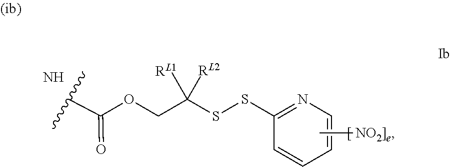

where R^{L1} and R^{L2} are independently selected from H and methyl, or together with the carbon atom to which they are bound form a cyclopropylene or cyclobutylene group; and
e is 0 or 1.

aP2 may be 0, 1, 2, 3, 4 or 5. In some instances, aP2 is 0 to 3. In some of these instances, aP2 is 0 or 1. In further instances, aP2 is 0.

b1P2 may be 0, 1, 2, 3, 4, 5, 6, 7, 8, 9, 10, 11, 12, 13, 14, 15 or 16. In some instances, b1P2 is 0 to 12. In some of these instances, b1P2 is 0 to 8, and may be 0, 2, 3, 4, 5 or 8.

b2P2 may be 0, 1, 2, 3, 4, 5, 6, 7, 8, 9, 10, 11, 12, 13, 14, 15 or 16. In some instances, b2P2 is 0 to 12. In some of these instances, b2P2 is 0 to 8, and may be 0, 2, 3, 4, 5 or 8.

In some instances, only one of b1P2 and b2P2 may not be 0.

cP2 may be 0 or 1.

dP2 may be 0, 1, 2, 3, 4 or 5. In some instances, dP2 is 0 to 3. In some of these instances, dP2 is 1 or 2. In further instances, dP2 is 2. In further instances, dP2 is 5.

In some instances of X^{P2}, aP2 is 0, b1P2 is 0, cP2 is 1 and dP2 is 2, and b2P2 may be from 0 to 8. In some of these instances, b2P2 is 0, 2, 3, 4, 5 or 8. In some instances of X^{P2}, aP2 is 1, b2P2 is 0, cP2 is 0 and dP2 is 0, and b1P2 may be from 0 to 8. In some of these instances, b1P2 is 0, 2, 3, 4, 5 or 8. In some instances of X^{P2}, aP2 is 0, b1P2 is 0, cP2 is 0 and dP2 is 1, and b2P2 may be from 0 to 8. In some of these instances, b2P2 is 0, 2, 3, 4, 5 or 8. In some instances of X², b1P2 is 0, b2P2 is 0, cP2 is 0 and one of aP2 and dP2 is 0. The other of aP2 and d is from 1 to 5. In some of these instances, the other of aP2 and d is 1. In other of these instances, the other of aP2 and dP2 is 5.

The instances for Q^{X} above for compounds of Formula I may apply to Q^{X} in Formula Ia^{P2} (e.g. where appropriate).

The instances for G^{L}, R^{L1}, R^{L2} and e above for compounds of Formula I may apply to compounds of Formula I^{P2}.

In some instances, the conjugate of formula IV is of the formula IV^{P2}:

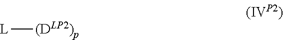

or a pharmaceutically acceptable salt or solvate thereof, wherein L is an antibody or antigen binding fragment thereof described herein (e.g. Ligand unit), D^{LP2} is a topoisomerase I inhibitor (e.g. Drug Linker unit) that is of formula III^{P2}:

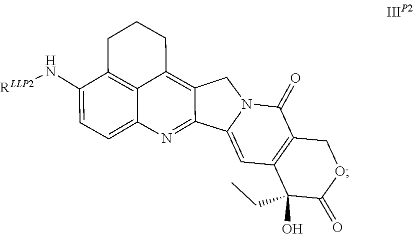

R^{LLP2} is a linker connected to the antibody or antigen binding fragment thereof (e.g. Ligand unit), wherein said linker is selected from

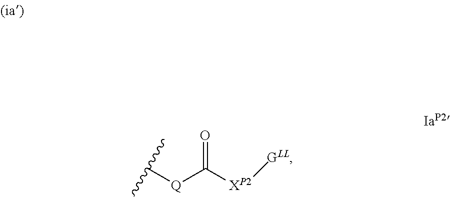

where Q and X^{P2} are as defined above and G^{LL} is a linker connected to the antibody or antigen binding fragment thereof; and

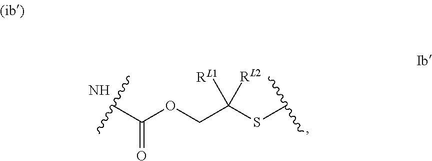

where R^{L1} and R^{L2} are as defined above; and
p is an integer of from 1 to 20.

Particularly suitable topoisomerase I inhibitors include those having the following formulas:

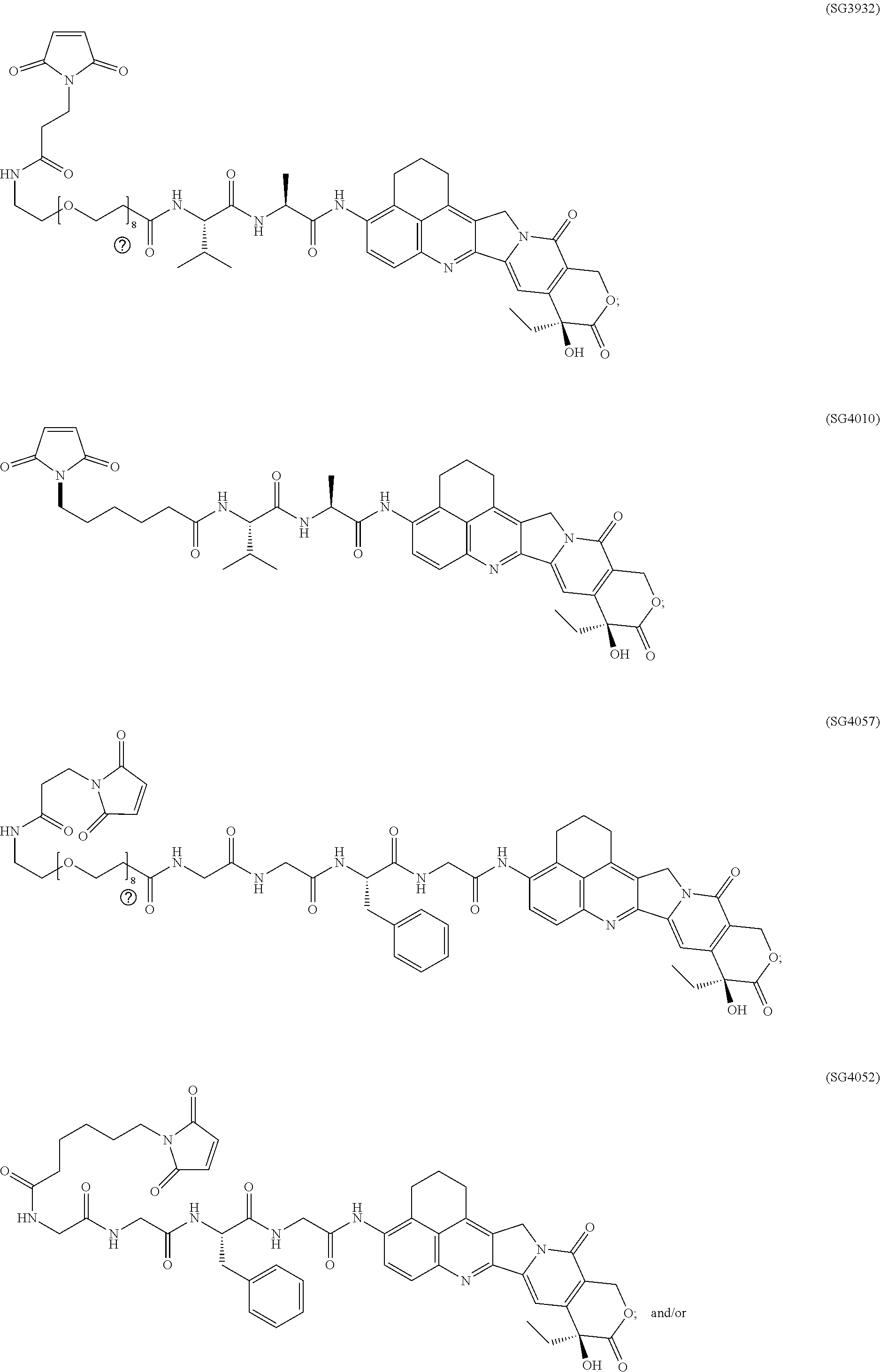

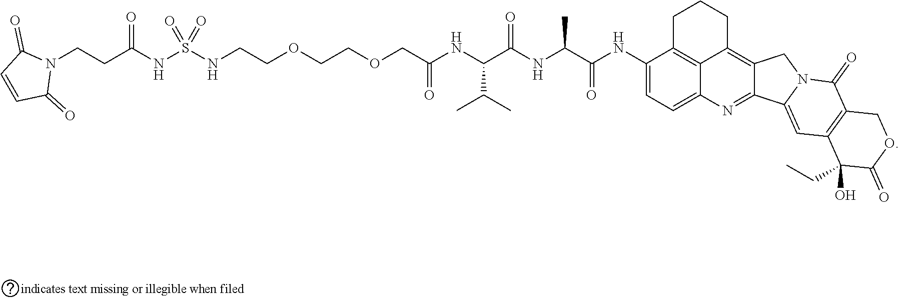 [SMILES: CC[C@@]1(O)C(=O)OCc2c1cc1n(c2=O)Cc2c-1nc1ccc(NC(=O)[C@H](C)NC(=O)[C@@H](NC(=O)COCCOCCNS(=O)(=O)NC(=O)CCN3C(=O)C=CC3=O)C(C)C)c3c1c2CCC3]

In some instances, an antibody molecule described herein is conjugated to a topoisomerase I inhibitor having the following formula (e.g. SG3932):

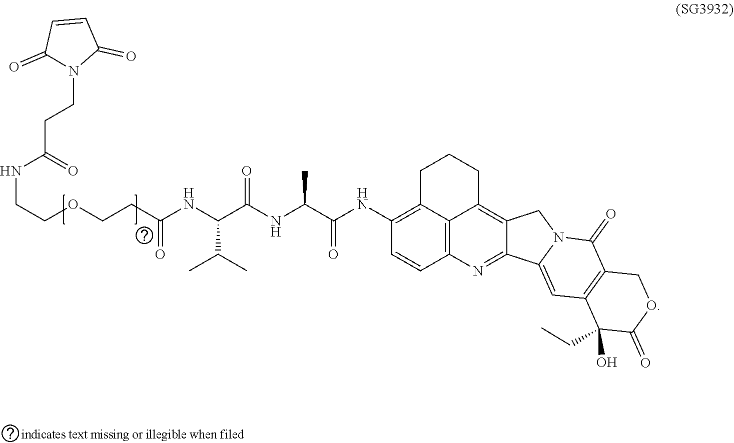

For the avoidance of doubt, the numeral ‘8’ specifies that the structure within boxed parentheses is repeated eight times. Thus, another representation of SG3932 is:

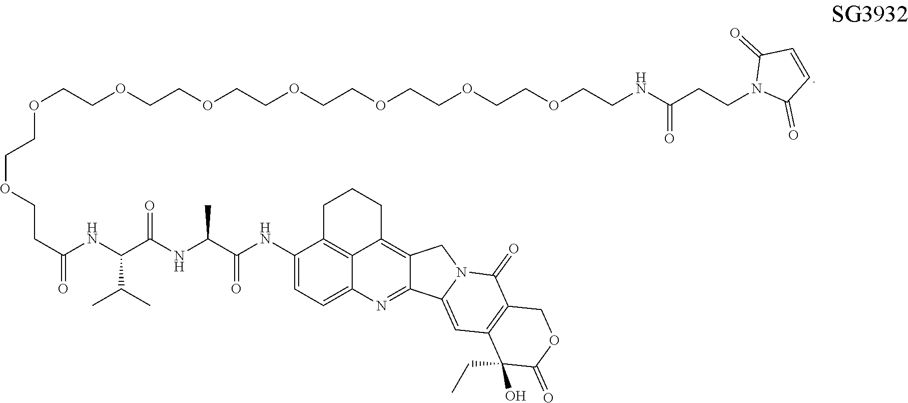 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

Another representation of SG4010 is:

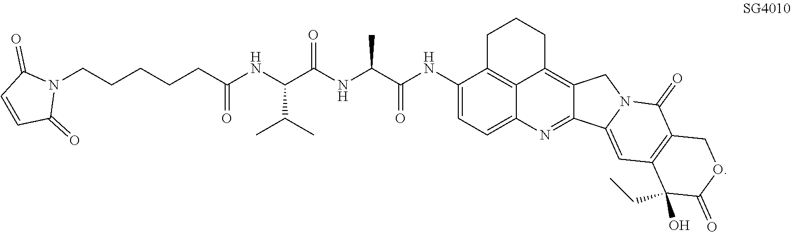 [SMILES: CC[C@@]1(O)C(=O)OCc2c1cc1n(c2=O)Cc2c-1nc1ccc(NC(=O)[C@H](C)NC(=O)[C@@H](NC(=O)CCCCCN3C(=O)C=CC3=O)C(C)C)c3c1c2CCC3]

Another representation of SG4057 is:

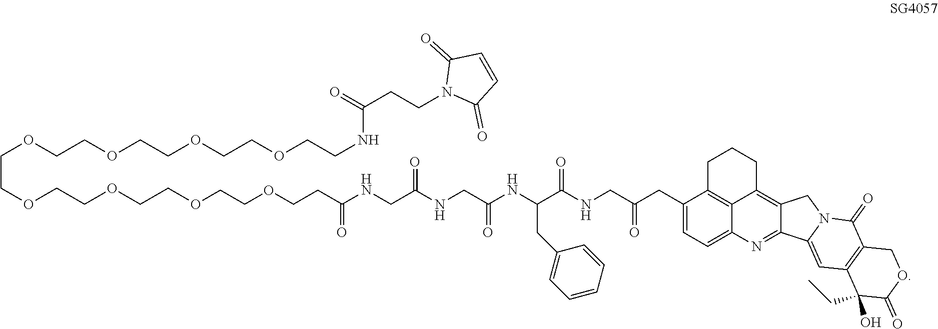 [SMILES: CC[C@@]1(O)C(=O)OCc2c1cc1n(c2=O)Cc2c-1nc1ccc(CC(=O)CNC(=O)C(Cc3ccccc3)NC(=O)CNC(=O)CNC(=O)CCOCCOCCOCCOCCOCCOCCOCCOCCNC(=O)CCN3C(=O)C=CC3=O)c3c1c2CCC3]

Another representation of SG4052 is:

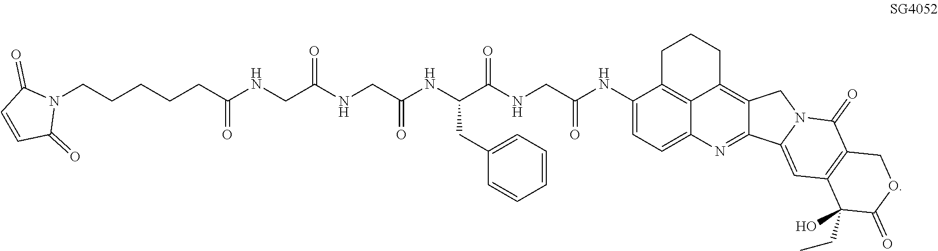 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCCCCN3C(=O)C=CC3=O)c3c1c2CCC3]

Any antibody or antigen binding fragment thereof described herein may be conjugated to one or more of said topoisomerase I inhibitor(s).

Synthesis of Topoisomerase I inhibitors

For completion, certain general synthetic routes for the preparation of an exemplary topoisomerase I inhibitor(s) will now be described.

Compounds of formula I where R^{L} is of formula Ia may be synthesised from a compound of Formula 2:

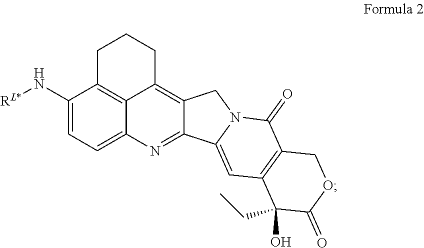

where R^{L*} is -QH by linking a compound of Formula 3:

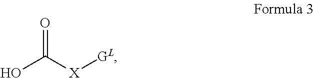

or an activated version thereof.

Such a reaction may be carried out under amide coupling conditions.

Compounds of Formula 2 may be synthesised by the deprotection of a compound of Formula 4:

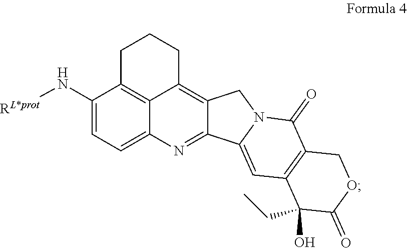

where R^{L*prot} is -Q-Prot^{N}, where Prot^{N} is an amine protecting group.

Compounds of Formula 4 may be synthesised by the coupling of a compound of Formula 5:

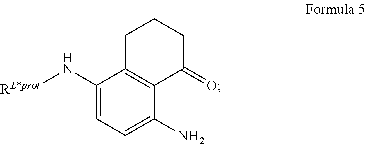

with the compound A3 using the Friedlander reaction.

Compounds of Formula 5 may be synthesised from compounds of Formula 6:

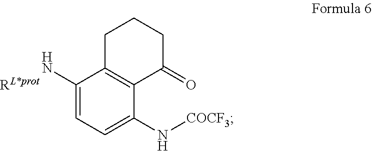

by removal of the trifluoroacetamide protecting group.

Compounds of Formula 6 may be synthesised by coupling: R^{L*Prot}-OH to the compound 17.

Compounds of formula I where R^{L} is of formula Ia or Ib may be synthesised from the compound Ill by coupling of the compound R^{L}-OH, or an activated form thereof.

### Amine Protecting Groups:

Amine protecting groups are well-known to those skilled in the art. Particular reference is made to the disclosure of suitable protecting groups in Greene's Protecting Groups in Organic Synthesis, Fourth Edition, John Wiley & Sons, 2007 (ISBN 978-0-471-69754-1), pages 696-871.

The drug loading (p) is the average number of drugs (e.g. tubulysin or topoisomerase inhibitor) per antibody molecule. In the compositions of the disclosure, drug loading ranges from 1 to 20 drugs (D) per antibody molecule. For example, drug loading may range from 1 to 10 drugs (D) per antibody molecule, i.e. where 1, 2, 3, 4, 5, 6, 7, 8, 9, or 10 drugs are covalently attached to the antibody molecule. Compositions of conjugates include collections of antibody molecules, conjugated with a range of drugs, from 1 to 10. Where the compounds of the disclosure are bound to lysines, drug loading may range from 1 to 80 drugs (D) per antibody molecule, although an upper limit of 40, 20, 10 or 8 may be preferred. Compositions of conjugates include collections of antibody molecules, conjugated with a range of drugs, from 1 to 80, 1 to 40, 1 to 20, 1 to 10 or 1 to 8.

The average number of drugs per antibody in preparations of conjugates from conjugation reactions may be characterized by conventional means such as UV, reverse phase HPLC, HIC, mass spectroscopy, ELISA assay, and electrophoresis. The quantitative distribution of conjugates in terms of p may also be determined. By ELISA, the averaged value of p in a particular preparation of conjugates may be determined (Hamblett, 2004; Sanderson, 2005). However, the distribution of p (drug) values is not discernible by the antibody-antigen binding and detection limitation of ELISA. Also, ELISA assay for detection of conjugates does not determine where the drug moieties are attached to the antibody molecule, such as the heavy chain or light chain fragments, or the particular amino acid residues. In some instances, separation, purification, and characterization of homogeneous conjugates where p is a certain value from conjugates with other drug loadings may be achieved by means such as reverse phase HPLC or electrophoresis. Such techniques are also applicable to other types of conjugates.

For some conjugates, p may be limited by the number of attachment sites on the antibody molecule. For example, an antibody molecule may have only one or several cysteine thiol groups, or may have only one or several sufficiently reactive thiol groups through which a linker may be attached.

Typically, fewer than the theoretical maximum of drug are conjugated to an antibody molecule during a conjugation reaction. An antibody molecule may contain, for example, many lysine residues that do not react with the linker (L). Only the most reactive lysine groups may react with an amine-reactive linker reagent. Also, only the most reactive cysteine thiol groups may react with a thiol-reactive linker reagent. Generally, antibody molecules do not contain many, if any, free and reactive cysteine thiol groups which may be linked to a drug moiety. Most cysteine thiol residues in the antibody molecules of the conjugates exist as disulfide bridges and must be reduced with a reducing agent such as dithiothreitol (DTT) or TCEP, under partial or total reducing conditions. The loading (drug/antibody ratio) of a conjugate may be controlled in several different manners, including: (i) limiting the molar excess of Drug Linker relative to antibody, (ii) limiting the conjugation reaction time or temperature, and (iii) partial or limiting reductive conditions for cysteine thiol modification.

Certain antibody molecules have reducible interchain disulfides, i.e. cysteine bridges. Antibody molecules may be made reactive for conjugation with linker reagents by treatment with a reducing agent such as DTT (dithiothreitol). Each cysteine bridge will thus form, theoretically, two reactive thiol nucleophiles. This process is also referred to as “classical conjugation” and is distinguished from methods such as where conjugation takes place at a cysteine that has been engineered into a specific site in the antibody molecule. Additional nucleophilic groups can be introduced into antibodies through the reaction of lysines with 2-iminothiolane (Traut's reagent) resulting in conversion of an amine into a thiol.

ADCs with drugs randomly conjugated to native cysteine residues are prepared by classical conjugation by partial reduction of the antibody followed by reaction with desired linker-drug. For example, the antibody at a concentration of 5 mg/mL may be partially reduced by addition of about 3 molar equivalents of DTT at pH 8.0, followed by incubation at about 37° C. for about 2 hours. The reduction reaction may then be chilled in ice and the excess DTT removed, for example, via diafiltration. The linker-drug can then be added at a linker-drug/thiol molar ratio of about 1:10. The conjugation reaction may be carried out in the presence of ˜10% v/v of DMSO. After conjugation, excess free cysteine (about 2 fold molar ratio over linker-drug) can be added to quench unreacted linker-drug to produce the cysteine-linker-drug adduct. The reaction mixture can then purified (e.g., by hydrophobic interaction chromatography), and subjected to buffer-exchange into PBS. Drug load distribution can be determined using standard methods, such as hydrophobic interaction chromatography and reduced reverse phase chromatography, as described elsewhere.

Methods to prepare conjugates using direct conjugation at solvent-accessible thiols generated by reduction of the antibody molecule interchain disulphide bridges involving N-alkyl maleimide are known. Other methods conjugate the drug at primary amines of lysines using N-hydroxysuccinimide ester. Such methods are reviewed in, for example, Gébleux and Casi, Pharmacol Ther (2016) 167: 48-59, which is herein incorporated by reference in its entirety.

Separately or in addition to the classical conjugation methods described above, it is also possible to use site-specific conjugation, a method in which drug load and site of conjugation is controlled. This can be achieved by, for example, engineering cysteines at specific residues, replacement of residues with unnatural amino acids with bio-orthogonal reactivity or enzyme ligation approaches. One method of site-specific conjugation is described in Dimasi, 2017, which is herein incorporated by reference in its entirety and involves inserting cysteines into antibody molecules at particular positions.

Cysteine amino acids may be engineered at reactive sites in an antibody molecule and which do not form intrachain or intermolecular disulfide linkages (Junutula, 2008; Doman, 2009; U.S. Pat. Nos. 7,521,541; 7,723,485; WO2009/052249). The engineered cysteine thiols may react with linkers or the drug-linker described herein which have thiol-reactive, electrophilic groups such as maleimide or alpha-halo amides to form conjugates with cysteine engineered antibody molecules and the drug. The location of the drug can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. If required, a drug loading near 2 can be achieved with near homogeneity of the conjugation product.

In some instances, the antibody molecule of the conjugate of the disclosure comprises a CH region and the drug is chemically conjugated at a cysteine amino acid inserted between positions 239 and 240 of the CH region, wherein the numbering of the constant region is as per the EU index. The connection between the antibody molecule and the drug may therefore be through this inserted cysteine amino acid and a terminal maleimide group on the linker.

Examples of CH regions that comprise a cysteine amino acid inserted between positions 239 and 240 of the CH region are SEQ ID NO: 43 and SEQ ID NO: 45. Examples of heavy chains comprising a CH region that comprises a cysteine amino acid inserted between positions 239 and 240 of the CH region are SEQ ID NOs: 50, 53 and 56.

In other instances, the antibody molecule of the conjugate does not comprise any amino acid residues inserted into the CH region. In particular instances, the antibody molecule of the conjugate does not comprise a cysteine amino acid inserted into the CH region (e.g. between positions 239 and 240, wherein the numbering of the constant region is as per the EU index). As demonstrated in the examples (e.g. Example 12), where classical conjugation is used to conjugate the drug-linker to native cysteines, the inserted cysteine is not necessary.

Examples of CH regions that do not comprise any amino acid residues inserted into the CH region are SEQ ID NO: 44, 46, 63 and 64. Examples of heavy chains comprising a CH region that do not comprise any amino acid residues inserted into the CH region are SEQ ID NOs: 51, 54, 57, 59 and 60.

Where more than one nucleophilic or electrophilic group of the antibody molecule reacts with a drug-linker intermediate, or linker reagent followed by drug reagent, then the resulting product is a mixture of conjugate compounds with a distribution of drug attached to an antibody, e.g. 1, 2, 3, etc. Liquid chromatography methods such as polymeric reverse phase (PLRP) and hydrophobic interaction (HIC) may separate compounds in the mixture by drug loading value. Preparations of conjugate with a single drug loading value (p) may be isolated, however, these single loading value conjugates may still be heterogeneous mixtures because the drug may be attached, via the linker, at different sites on the antibody molecule.

Thus the conjugate compositions of the disclosure include mixtures of antibody-drug conjugate compounds where the antibody has one or more drug moieties (e.g. tubulysin or topoisomerase inhibitor) and where the drug moieties may be attached to the antibody molecule at various amino acid residues.

In some instances, the average number of tubulysin drug moieties per antibody molecule is in the range 1 to 8. In some instances the range is selected from 1 to 6, 1 to 4, 1 to 3, optionally 1 to 2, 1.5 to 2, 1.8 to 2, such as 1.9 to 2.

As already described above, in some instances the antibody molecule of the ADC may comprise one or more mutations in the CH region(s) of the heavy chain(s) to reduce or abrogate binding of the antibody molecule to one or more Fcγ receptors. Thus, the first and/or second heavy chain of the ADCs described herein may comprise phenylalanine (F) at position 234, glutamic acid (E) at position 235, and serine (S) at position 331, wherein the numbering is as per the EU index.

### Functional Properties of the Antibody Molecules and Conjugates

The antibody molecules and conjugates described herein may be characterised by reference to certain functional properties.

### Binding Affinity

The antibody molecules and conjugates described herein may be characterised by the antigen-binding domain that binds EGFR having a particular affinity for EGFR and/or the antigen-binding domain that binds c-Met having a particular affinity for c-Met. The binding affinity of an antibody molecule to a cognate antigen, such as human, mouse or cynomolgus EGFR or c-Met can be determined by surface plasmon resonance (SPR), using Biacore, for example. The binding affinity can be determined using an antibody molecule, for example as part of a bispecific antibody molecule that comprises a first antigen-binding domain that binds EGFR and a second antigen-binding domain that binds c-Met. Alternatively, the binding affinity can be determined using an antibody molecule that is monospecific for EGFR or c-Met. In some instances, the binding affinity is determined using BIACore as described in Example 2.1.

Binding affinity is typically measured by Kd (the equilibrium dissociation constant between the antigen-binding domain and its antigen). As is well understood, the lower the Kd value, the higher the binding affinity of the antigen-binding domain. For example, an antigen-binding domain that binds to a target with a Kd of 10 nM would be considered to be binding said target with a higher affinity than an antigen-binding domain that binds to the same target with a Kd of 100 nM.

Reference to human EGFR may refer to a polypeptide comprising the extracellular domain of EGFR, such as one having the amino acid sequence set forth in SEQ ID NO: 68. Reference to mouse EGFR may refer to a polypeptide produced from the molecule available from SinoBiological with catalogue #51091-M08H. Reference to cynomolgus EGFR may refer to the amino acid sequence set forth in SEQ ID NOs: 69. Reference to human c-Met may refer to a polypeptide having the amino acid sequence set forth in SEQ ID NO: 70. Reference to mouse c-Met may refer to a polypeptide having the amino acid sequence set forth in SEQ ID NO: 90 or to the polypeptide produced from the molecule available from SinoBiological with catalogue #50622-M08H. Reference to cynomolgus c-Met may refer to the amino acid sequence set forth in SEQ ID NO: 71.

### EGFR Affinity

Antibody molecules and conjugates described herein may comprise a binding domain that binds to EGFR with a low affinity. EGFR is known to be expressed at low levels in normal tissues, e.g. the skin e.g. the skin. Antibody molecules and conjugates that bind to EGFR with a low-affinity are advantageously expected to display reduced on-target toxicity in normal tissues whilst still being able to target tumors expressing high levels of EGFR, resulting in an improved safety profile.

Furthermore, as demonstrated herein, conjugates comprising this low affinity EGFR binding domain are more efficacious at treating cancer compared to conjugates comprising a higher affinity EGFR binding domain.

The binding domain that binds to EGFR may bind to human EGFR with an affinity having a Kd equal to or higher than 10 nM, 15 nM, 20 nM, 25 nM, 30 nM, 35 nM, or 40 nM. Alternatively, binding domain that binds to EGFR may bind to human EGFR with a Kd of between 10 and 100 nM, between 20 and 100 nM, between 30 and 100 nM, between 40 and 100 nM, between 10 and 80 nM, between 20 and 80 nM, between 30 and 80 nM, between 40 and 80 nM, between, between 10 and 70 nM, between 20 and 70 nM, between 30 and 70 nM, between 40 and 70 nM, between 10 and 60 nM, between 20 and 60 nM, between 30 and 60 nM, between 40 and 60 nM, between 10 and 50 nM, between 20 and 50 nM, between 30 and 50 nM, or between 40 and 50 nM.

The binding domain that binds to EGFR may bind to human EGFR with an affinity that is lower than the affinity that a binding domain comprising the heavy chain sequence and light chain sequence of antibody molecule QD6 set forth in SEQ ID NOs: 53 and 55, respectively.

For example, the binding domain that binds to EGFR may bind to human EGFR with an affinity having a Kd that is at least 2-fold, 3-fold, 4-fold, 5-fold, 6-fold, or 7-fold higher than the Kd that a binding domain comprising the heavy chain sequence and light chain sequence of antibody molecule QD6 set forth in SEQ ID NOs: 53 and 55, respectively, binds human EGFR. Alternatively, the binding domain that binds to EGFR may bind to human EGFR with an affinity having a Kd that is between 2- and 10-fold higher, between 3- and 10-fold higher, between 4- and 10-fold higher, between 5- and 10-fold higher, between 6- and 10-fold higher, between 7- and 10-fold higher, between 2- and 9-fold higher, between 3- and 9-fold higher, between 4- and 9-fold higher, between 5- and 9-fold higher, between 6- and 9-fold higher, between 7- and 9-fold higher, between 2- and 8-fold higher, between 3- and 8-fold higher, between 4- and 8-fold higher, between 5- and 8-fold higher, between 6- and 8-fold higher, between 7- and 8-fold higher than the Kd that a binding domain comprising the heavy chain sequence and light chain sequence of antibody QD6 set forth in SEQ ID NOs: 53 and 55, respectively, binds human EGFR.

The binding domain that binds to EGFR may bind to human EGFR with an affinity that is similar to the affinity that a binding domain comprising the variable heavy region sequence and variable light region sequence of antibody molecule RAA22 set forth in SEQ ID NOs: 16 and 20, respectively binds human EGFR. For example, the binding domain that binds to EGFR may bind to human EGFR with an affinity having a Kd that is less than 5-fold different, less than 4-fold different, less than 3-fold different, less than 2-fold different, less than 1-fold different or less than 0.5-fold different than an binding domain comprising the variable heavy region sequence and variable light region sequence of antibody molecule RAA22 set forth in SEQ ID NOs: 16 and 20, respectively, binds human EGFR.

The binding domain that binds to EGFR may also bind to cynomolgus EGFR. For example, the binding domain that binds to EGFR may bind to cynomolgus EGFR with an affinity having a Kd that is less than 700 nM, less than 600 nM, less than 500 nM, less than 400 nM, less than 300 nM, or less than 250 nM. Alternatively, the antigen-binding domain that binds to EGFR may bind to cynomolgus EGFR with an affinity having a Kd of between 100 and 700 nM, between 100 and 600 nM, between 100 and 500 nM, between 100 and 400 nM, between 100 and 300 nM, between 150 and 250 nM, between 100 and 200 nM. The antigen-binding domain that binds to EGFR may bind to cynomolgus EGFR with a Kd that is less than or equal 10-, 9-, 8-, 7-, 6-, 5-, 4-, 3-fold higher Kd than the binding domain binds to human EGFR.

The binding domain that binds to EGFR may also bind to mouse EGFR. For example, the binding domain that binds to EGFR may bind to mouse EGFR with an affinity having a Kd that is less than 1 μM, less than 900 nM, less than 800 nM, less than 700 nM, less than 600 nM or less than 650 nM. Alternatively, the binding domain that binds to EGFR may bind to mouse EGFR with a Kd of between 100 nM and 1 μM, between 200 and 900 nM, between 300 and 800 nM, between 400 and 700 nM, between 400 and 600 nM, or between 450 and 550 nM.

In some instances, the binding domain that binds to EGFR is capable of binding human EGFR and cynomolgus EGFR. This cross-reactivity is advantageous, as it allows dosing and safety testing of the antibody molecules and conjugates to be performed in cynomolgus monkeys during preclinical development. In some instances, the binding domain that binds to EGFR is capable of binding human EGFR, cynomolgus EGFR and mouse EGFR. For example, the binding domain that binds EGFR may be capable of binding human EGFR, cynomolgus EGFR and mouse EGFR with the Kd values set out above (e.g. human EGFR with a Kd of between 10 and 100 nM, cynomolgus EGFR with a Kd of between 100 and 700 nM and mouse EGFR with a Kd of between 100 nM and 1 μM).

cMET affinity

The binding domain that binds to cMET may bind to human cMET with an affinity having a Kd of lower than 20 nM, 15 nM, 12 nM, 11 nM, 10 nM, 9 nM, 8 nM, 7 nM, 6 nM, 5 nM, 4 nM, 3 nM or 2.5 nM. Alternatively, antigen-binding domain that binds to cMET may bind to human c-Met with an affinity having a Kd of between 1 and 20 nM, between 1 and 15 nM, between 1 and 10 nM, between 1 and 9 nM, between 1 and 8 nM, between 1 and 7 nM, between 1 and 6 nM, between 1 and 5 nM, between 1 and 4 nM, between 1 and 3 nM, between 1 and 2.5 nM, or between 2 and 2.5 nM.

The binding domain that binds to cMET may bind to cynomolgus cMET. For example, the binding domain that binds to cMET may bind to cynomolgus cMET with an affinity having a Kd that is lower than 30 nM, 25 nM, 20 nM, 15 nM, 10 nM, 9 nM, 8 nM, 7 nM, 6 nM, 5 nM, 4 nM, 3 nM, or 2.5 nM. Alternatively, the binding domain that binds to cMET may bind to cynomolgus cMET with an affinity having a Kd of between 1 and 20 nM, between 1 and 15 nM, between 1 and 10 nM, between 1 and 9 nM, between 1 and 8 nM, between 1 and 7 nM, between 1 and 6 nM, between 1 and 5 nM, between 1 and 4 nM, between 1 and 3 nM, between 1 and 2.5 nM, or between 2 and 2.5 nM. The binding domain that binds to cMET may bind to cynomolgus cMET with an affinity having a Kd that is less than or equal 10-, 9-, 8-, 7-, 6-, 5-, 4-, 3-, 2-, 1-fold higher Kd than the antigen-binding domain binds to human c-Met.

In some instances, the binding domain that binds to cMET is capable of binding human cMET and cynomolgus cMET. This cross-reactivity is advantageous, as it allows dosing and safety testing of the antibody molecules to be performed in cynomolgus monkeys during preclinical development. For example, the binding domain that binds cMET may be capable of binding human cMET and cynomolgus cMET with the Kd values set out above (e.g. human cMET with a Kd of between 1 and 20 nM and cynomolgus cMET with a Kd of between 1 and 20 nM).

### Specific Binding

The binding domains described herein may specifically bind their respective targets (i.e. EGFR and cMET). The term “specific” may refer to the situation in which the antigen-binding domain will not show any significant binding to molecules other than its specific binding partner(s), here EGFR or cMET. Such molecules are referred to as “non-target molecules”. The term “specific” is also applicable where the antibody molecule is specific for particular epitopes, such as epitopes on EGFR or cMET, that are carried by a number of antigens in which case the antibody molecule will be able to bind to the various antigens carrying the epitope.

In some instances, an antibody molecule is considered to not show any significant binding to a non-target molecule if the extent of binding to a non-target molecule is less than about 10% of the binding of the antibody to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the antibody molecule described herein binds to EGFR and/or c-Met with an affinity that is at least 0.1 order of magnitude greater than the affinity towards another, non-target molecule. In some instances, the antibody molecule of the present disclosure binds to EGFR and/or cMET with an affinity that is one of at least 0.2, 0.3, 0.4, 0.5, 0.6, 0.7, 0.8, 0.9, 1.0, 1.5, or 2.0 orders of magnitude greater than the affinity towards another, non-target molecule.

EGFR is a member of the ErbB family of receptors, a subfamily of four closely related receptor tyrosine kinases: EGFR, HER2, HER3 and HER4. The RAA22 antigen-binding domain showed no binding to HER2, HER3 and HER4, demonstrating that this antigen-binding domain binds EGFR specifically. Thus, in a particular instance, the antigen-binding domain that binds EGFR does not bind, or does not show any significant binding, to HER2, HER3 or HER4.

cMET is a member of the subfamily of receptor tyrosine kinases that includes Ron and Sema 4a. The B09-GL antigen-binding domain showed no binding to Ron and Sema 4a, demonstrating that this antigen-binding domain binds cMET specifically. Thus, in a particular instance, the binding domain that binds cMET does not bind, or does not show any significant binding, to Ron, Sema 4a.

### Concurrent Engagement

The antibody molecules and conjugates described herein may be characterised by the ability of both the binding domains to concurrently engage their respective EGFR and cMET targets. Antibody molecules and conjugates with the ability to concurrently engage EGFR and cMET are expected to be advantageous, as numerous tumours are known to co-express both EGFR and c-Met and therefore can be targeted by antibody molecules of the disclosure. Thus, in some instances the antibody molecule is able to concurrently engage EGFR and cMET.

Concurrent engagement can be determined for example by an in vitro cytotoxicity assay using a cell line expresses roughly equal amounts of EGFR and cMET and a conjugate comprising the antibody molecules with EGFR and cMET antigen-binding domains. If the individual antigen-binding domains in the conjugate function independently to deliver the drug, blocking either target in this cell line would be expected to only modestly reduce the activity of the conjugate, shifting the IC50 by 2-fold or less, since the targets are present at similar levels. The EGFR and cMET targets can be blocked in this assay by using, for example, a monospecific antibody molecule that binds the same region on either EGFR or cMET, but lacks a drug that is able to induce cytotoxicity. For example, the monospecific antibody molecule may contain the same antigen-binding domain that binds EGFR, or the same antigen-binding domain that binds c-Met, as the conjugate being tested. If, on the other hand, the conjugate requires concurrent engagement to effectively deliver the conjugate into cells, blocking either target would be likely to have a greater impact on the activity of the conjugate. That is, the antibody molecule is considered to be able to engage both targets concurrently if there is a shift in IC50 by at least 2-fold, at least 5-fold, or at least 10-fold after blocking either target when using this assay.

An additional method to determine concurrent engagement is to compare the activity of the bispecific EGFR—cMET conjugate to monovalent, monospecific control conjugates in an in vitro cytotoxicity assay. The control conjugates comprise one antigen-binding domain to either EGFR or c-Met and one non-binding isotype antibody control antigen-binding domain. If each antigen-binding domain in the bispecific conjugate functions independently, the expected result would be that each monospecific control conjugate would only be modestly less potent than the bispecific conjugate, and the difference would be additive. Alternatively, if the two antigen-binding domains of the bispecific conjugate function synergistically by concurrent binding, one would expect larger differences in activity of the bispecific conjugate compared to the monospecific control antibodies. That is, the antibody molecule is considered to be able to engage both targets concurrently if the bispecific conjugate results in a shift in IC50 that is greater than the sum of the shifts in IC50 observed using the monospecific control conjugates.

Further details of these methods to measure concurrent engagement can be found in the examples.

### Antibody Internalisation

The antibody molecules and conjugates described herein may be characterised by their ability to mediate efficient internalisation. This is particularly useful for conjugates, as it ensures the conjugate is internalised into the cell and delivered to lysosomes, where the antibody molecule is subsequently degraded and drug released into the cell, where it exerts its cellular effects, e.g. cytotoxicity.

Internalisation of an antibody molecule by cells can be analysed by contacting live cells with the antibody molecule, and detecting the antibody molecule after sufficient period of time for internalisation. Internalisation can be determined by detection of the localisation of the antibody molecule. Where the antibody molecule remains on the surface of the cell (e.g. is detected on the cell surface, and/or is not detected inside the cell), the antibody molecule is determined not to have been internalised. Where the antibody molecule is detected inside the cell (e.g. localised to the cytoplasm or a cellular organelle), the antibody molecule is determined to have been internalised.

An exemplary method for visualising whether the antibody molecule is able to mediate efficient internalisation involves labelling the antibody molecule with pH sensitive dyes that exhibit fluorescent at an acidic pH and adding these labelled antibody molecules to cells. Internalisation into the cell can be detected by monitoring fluorescence. The antibody molecule is considered able to mediate internalisation and delivery to lysosomes if the fluorescence observed is greater than that of a labelled non-binding control antibody molecule over a certain time period, for example 48 hours. Further details of this method to visualise antibody internalisation can be found in the examples.

The antibody molecules described herein may be characterised by their ability to mediate more efficient internalisation when compared to the EGFR or cMET monospecific controls. Antibody molecules and conjugates that exhibit this properties are expected to be advantageous, as they are expected to display greater selectivity to tumour cells co-expressing both targets and could minimise the impact of the antibody molecule in normal tissues that do not display significant levels of co-expression.

### In Vitro Activity

The antibody molecules described herein may be characterised by their cytotoxic activity, i.e. their ability to kill cells. Cytotoxic activity can be measured using an in vitro cell viability assay, such as the CellTiter-Glo® (Promega) assay, for example. In some instances, the cells are cells that expression both EGFR and cMET.

Potency of an antibody molecule can be expressed as an IC50 value. IC50 is the median inhibitory concentration of an antibody molecule. In functional assays, IC50 is the concentration that reduces a biological response by 50% of its maximum. IC50 can be calculated by any number of means known in the art.

In some instances, the antibody molecules described herein having cytotoxic activity have an IC50 of less than less than 4000 μM, less than 3500 μM, less than 3000 μM, less than 2500 μM, less than 2000 μM, less than 1500 μM, less than 1000 μM, less than 500 μM, less than 400 μM, less than 300 μM, less than 250 μM, less than 200 μM, less than 150 μM, or less than 100 μM when measured using an in vitro cell viability assay. In some instances, the antibody molecules described herein may have an IC50 of between 60 and 500 μM.

In some instances, the antibody molecules described herein are capable of increased killing of cells, e.g. tumor cells, that express significant amounts of both EGFR and cMET compared to cells that express low levels of one or the other of EGFR and cMET. Cells expressing significant amounts of both EGFR and cMET may be determined by measuring relative receptor density at the cell surface. For example, cells expressing EGFR and cMET at a relative receptor density at the cell surface of greater than 15,000 may be considered cells that express significant amounts of both EGFR and cMET and cells that express one of EGFR and c-Met at a low relative receptor density at the cell surface of 15,000 or less. Relative EGFR and cMET density can be measured, for example, using the Quantum MESF quantitative FACS assay kit as described in the examples.

Examples of cells that express significant amounts of both EGFR and cMET may include NCI H596, HCC 827 GR Pool, A549, NCI H1792, NCI H1975, NCI H292 and NCI H358 cell lines. Examples of cells that express one of EGFR and cMET at a low relative receptor density may include A427, NCI H23 and NCI H661 (Ag negative) cell lines. These cell lines are available through ATCC.

### EGFR Tyrosine Kinase Inhibitors

EGFR TKIs can be characterised as either first-, second- or third-generation EGFR TKIs, as set out below.

First-generation EGFR TKIs are reversible inhibitors of EGFR bearing activating mutations that do not significantly inhibit EGFR bearing the T790M mutation. Examples of first-generation TKIs include gefitinib and erlotinib.

Second-generation EGFR TKIs are irreversible inhibitors of EGFR bearing activating mutations that do not significantly inhibit EGFR bearing the T790M mutation. Examples of second-generation TKIs include afatinib and dacomitinib.

Third-generation EGFR TKIs are inhibitors of EGFR bearing activating mutations that also significantly inhibit EGFR bearing the T790M mutation and do not significantly inhibit wild-type EGFR. Examples of third-generation TKIs include compounds of Formula (V), osimertinib, AZD3759 (zorifertinib), lazertinib, nazartinib (EGF816), C01686 (rociletinib), HM61713, ASP8273 (naquotinib), PF-06747775 (mavelertinib), avitinib (abivertinib), alflutinib (AST2818) and CX-101 (olafertinib; RX-518), almonertinib (HS-10296; aumolertinib) and BPI-7711 (rezivertinib).

In an aspect, the EGFR TKI is a first-generation EGFR TKI. In further instances, the first-generation EGFR TKI is selected from the group consisting of gefitinib or a pharmaceutically acceptable salt thereof, icotinib or a pharmaceutically acceptable salt thereof, and erlotinib or a pharmaceutically acceptable salt thereof.

In an aspect, the EGFR TKI is a second-generation EGFR TKI. In a further aspect, the second-generation EGFR TKI is selected from dacomitinib, or a pharmaceutically acceptable salt thereof, and afatinib or a pharmaceutically acceptable salt thereof.

In an aspect, the EGFR TKI is a third-generation EGFR TKI. In a further aspect, the third-generation EGFR TKI is a compound of Formula (V), as defined below. In a further aspect, the third-generation EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, CX-101 or a pharmaceutically acceptable salt thereof, HS-10296 or a pharmaceutically acceptable salt thereof and BPI-7711 or a pharmaceutically acceptable salt thereof. In a further aspect, the third generation EGFR TKI is osimertinib or a pharmaceutically acceptable salt thereof.

### Compounds of Formula (V)

In an aspect, the EGFR TKI is a compound of Formula (V):

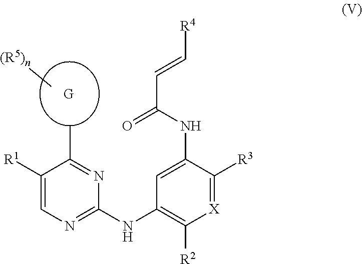

wherein:
G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;
R¹ is selected from hydrogen, fluoro, chloro, methyl and cyano;
R² is selected from methoxy, trifluoromethoxy, ethoxy, 2,2,2-trifluoroethoxy and methyl;
R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;
R⁴ is selected from hydrogen, 1-piperidinomethyl and N,N-dimethylaminomethyl;
R⁵ is independently selected from methyl, ethyl, propyl, 2,2-difluoroethyl, 2,2,2-trifluoroethyl, fluoro, chloro and cyclopropyl;
X is CH or N; and
n is 0, 1 or 2;
or a pharmaceutically acceptable salt thereof.

In a further aspect there is provided a compound of Formula (V), as defined above, wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from[2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

Examples of compounds of Formula (V) include those described in WO 2013/014448, WO 2015/175632, WO 2016/054987, WO 2016/015453, WO 2016/094821, WO 2016/070816 and WO 2016/173438.

### Osimertinib and Pharmaceutical Compositions Thereof

Osimertinib has the following chemical structure:

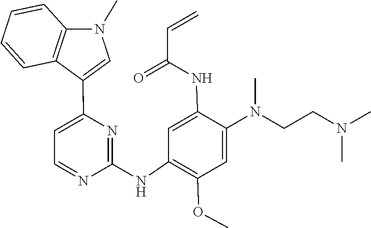 [SMILES: C=CC(=O)Nc1cc(Nc2nccc(-c3cn(C)c4ccccc34)n2)c(OC)cc1N(C)CCN(C)C]

The free base of osimertinib is known by the chemical name: N-(2-{2-dimethylamino ethyl-methylamino}-4-methoxy-5-{[4-(1-methylindol-3-yl)pyrimidin-2-yl]amino}phenyl) prop-2-enamide. Osimertinib is described in WO 2013/014448. Osimertinib is also known as AZD9291.

Osimertinib may be found in the form of the mesylate salt: N-(2-{2-dimethylamino ethyl-methylamino}-4-methoxy-5-{[4-(1-methylindol-3-yl)pyrimidin-2-yl]amino}phenyl) prop-2-enamide mesylate salt. Osimertinib mesylate is also known as TAGRISSO™.

Osimertinib mesylate is currently approved as an oral once daily tablet formulation, at a dose of 80 mg (expressed as free base, equivalent to 95.4 mg osimertinib mesylate), for the treatment of metastatic EGFR T790M mutation positive NSCLC patients. A 40 mg oral once daily tablet formulation (expressed as free base, equivalent to 47.7 mg osimertinib mesylate) is available should dose modification be required. The tablet core comprises pharmaceutical diluents (such as mannitol and microcrystalline cellulose), disintegrants (such as low-substituted hydroxypropyl cellulose) and lubricants (such as sodium stearyl fumarate). The tablet formulation is described in WO 2015/101791.

In an aspect, therefore, osimertinib, or a pharmaceutically acceptable salt thereof, is in the form of the mesylate salt, i.e. N-(2-{2-dimethylamino ethyl-methylamino}-4-methoxy-5-{[4-(1-methylindol-3-yl)pyrimidin-2-yl]amino}phenyl) prop-2-enamide mesylate salt.

In an aspect, osimertinib, or a pharmaceutically acceptable salt thereof, is administered once-daily. In a further aspect, osimertinib mesylate is administered once-daily.

In an aspect, the total daily dose of osimertinib is about 80 mg. In a further aspect, the total daily dose of osimertinib mesylate is about 95.4 mg.

In an aspect, the total daily dose of osimertinib is about 40 mg. In a further aspect, the total daily dose of osimertinib mesylate is about 47.7 mg.

In an aspect, osimertinib, or a pharmaceutically acceptable salt thereof, is in tablet form.

In an aspect, osimertinib, or a pharmaceutically acceptable salt thereof, is administered in the form of a pharmaceutical composition comprising one or more pharmaceutically acceptable excipients (for example a diluent or carrier). In a further aspect, the composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as sodium stearyl fumarate).

In an aspect, the composition is in the form of a tablet, wherein the tablet core comprises: (a) from 2 to 70 parts of osimertinib, or a pharmaceutically acceptable salt thereof; (b) from 5 to 96 parts of two or more pharmaceutical diluents; (c) from 2 to 15 parts of one or more pharmaceutical disintegrants; and (d) from 0.5 to 3 parts of one or more pharmaceutical lubricants; and wherein all parts are by weight and the sum of the parts (a)+(b)+(c)+(d)=100.

In an aspect, the composition is in the form of a tablet, wherein the tablet core comprises: (a) from 7 to 25 parts of osimertinib, or a pharmaceutically acceptable salt thereof; (b) from 55 to 85 parts of two or more pharmaceutical diluents, wherein the pharmaceutical diluents comprise microcrystalline cellulose and mannitol; (c) from 2 to 8 parts of pharmaceutical disintegrant, wherein the pharmaceutical disintegrant comprises low-substituted hydroxypropyl cellulose; (d) from 1.5 to 2.5 parts of pharmaceutical lubricant, wherein the pharmaceutical lubricant comprises sodium stearyl fumarate; and wherein all parts are by weight and the sum of the parts (a)+(b)+(c)+(d)=100.

In an aspect, the composition is in the form of a tablet, wherein the tablet core comprises: (a) about 19 parts of osimertinib mesylate; (b) about 59 parts of mannitol; (c) about 15 parts of microcrystalline cellulose; (d) about 5 parts of low-substituted hydroxypropyl cellulose; and (e) about 2 parts of sodium stearyl fumarate; and wherein all parts are by weight and the sum of the parts (a)+(b)+(c)+(d)+(e)=100.

AZD3759 (zorifertinib)

AZD3759 has the following chemical structure:

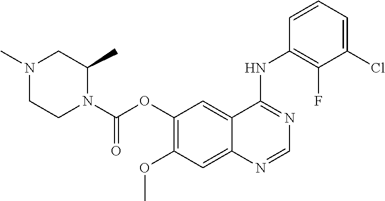 [SMILES: COc1cc2ncnc(Nc3cccc(Cl)c3F)c2cc1OC(=O)N1CCN(C)C[C@H]1C]

The free base of AZD3759 is known by the chemical name: 4-[(3-chloro-2-fluorophenyl)amino]-7-methoxy-6-quinazolinyl (2R)-2,4-dimethyl-1-piperazinecarboxylate. AZD3759 is described in WO 2014/135876.

In an aspect, AZD3759, or a pharmaceutically acceptable salt thereof, is administered twice-daily. In a further aspect, AZD3759 is administered twice-daily.

In an aspect, the total daily dose of AZD3759 is about 400 mg. In a further aspect, about 200 mg of AZD3759 is administered twice a day.

### Lazertinib

Lazertinib has the following chemical structure:

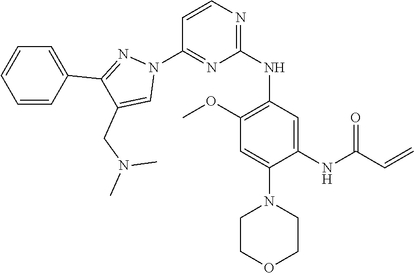 [SMILES: C=CC(=O)Nc1cc(Nc2nccc(-n3cc(CN(C)C)c(-c4ccccc4)n3)n2)c(OC)cc1N1CCOCC1]

The free base of lazertinib is known by the chemical name N-{5-[(4-{4-[(dimethylamino)methyl]-3-phenyl-1H-pyrazol-1-yl}-2-pyrimidinyl)amino]-4-methoxy-2-(4-morpholinyl)phenyl}acrylamide. Lazertinib is described in WO 2016/060443. Lazertinib is also known by the names YH25448 and GNS-1480.

In an aspect, lazertinib, or a pharmaceutically acceptable salt thereof, is administered once-daily. In a further aspect, lazertinib is administered once-daily.

In an aspect, the total daily dose of lazertinib is about 20 to 320 mg.

In an aspect, the total daily dose of lazertinib is about 240 mg.

### Avitinib

Avitinib has the following chemical structure:

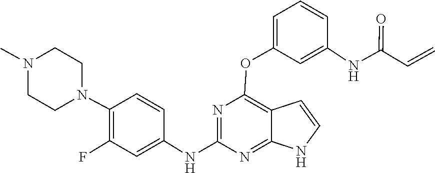 [SMILES: C=CC(=O)Nc1cccc(Oc2nc(Nc3ccc(N4CCN(C)CC4)c(F)c3)nc3[nH]ccc23)c1]

The free base of avitinib is known by the chemical name: N-(3-((2-((3-fluoro-4-(4-methylpiperazin-1-yl)phenyl)amino)-7H-pyrrolo(2,3-d)pyrimidin-4-yl)oxy)phenyl)prop-2-enamide. Avitinib is disclosed in US2014038940. Avitinib is also known as abivertinib.

In an aspect, avitinib or a pharmaceutically acceptable salt thereof, is administered twice daily. In a further aspect, avitinib maleate is administered twice daily.

In an aspect, the total daily dose of avitinib maleate is about 600 mg.

### Alflutinib

Alflutinib has the following chemical structure:

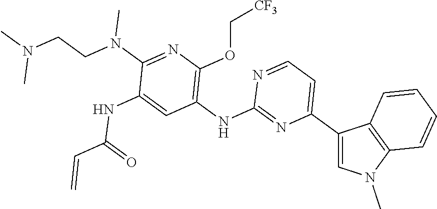 [SMILES: C=CC(=O)Nc1cc(Nc2nccc(-c3cn(C)c4ccccc34)n2)c(OCC(F)(F)F)nc1N(C)CCN(C)C]

The free base of alflutinib is known by the chemical name: N-{2-{[2-(dimethylamino)ethyl](methyl)amino}-6-(2,2,2-trifluoroethoxyl)-5-{[4-(1-methyl-1H-indol-3-yl)pyrimidin-2-yl]amino}pyridin-3-yl}acrylamide. Alflutinib is disclosed in WO 2016/15453. Alflutinib is also known as AST2818.

In an aspect, alflutinib or a pharmaceutically acceptable salt thereof, is administered once daily. In a further aspect, alflutinib mesylate is administered once daily.

In an aspect, the total daily dose of alflutinib mesylate is about 80 mg.

In an aspect, the total daily dose of alflutinib mesylate is about 40 mg.

### Afatinib

Afatinib has the following chemical structure:

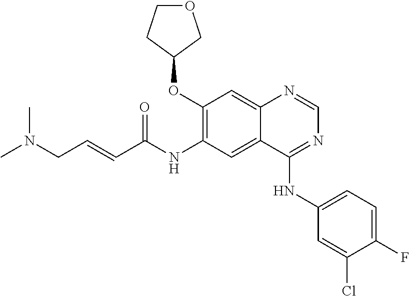 [SMILES: CN(C)C/C=C/C(=O)Nc1cc2c(Nc3ccc(F)c(Cl)c3)ncnc2cc1O[C@H]1CCOC1]

The free base of afatinib is known by the chemical name: N-[4-(3-chloro-4-fluoroanilino)-7-[(3S)-oxolan-3-yl]oxyquinazolin-6-yl]-4-(dimethylamino)but-2-enamide. Afatinib is disclosed in WO 02/50043. Afatinib is also known as Gilotrif.

In an aspect, afatinib or a pharmaceutically acceptable salt thereof, is administered once daily. In a further aspect, afatinib dimaleate is administered once daily.

In an aspect, the total daily dose of afatinib dimaleate is about 40 mg.

In an aspect, the total daily dose of afatinib dimaleate is about 30 mg.

CX-101 (olafertinib; RX-518)

CX-101 has the following chemical structure:

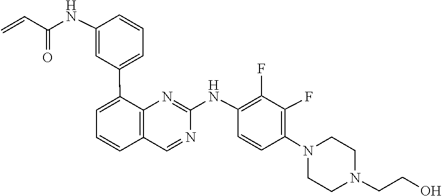 [SMILES: C=CC(=O)Nc1cccc(-c2cccc3cnc(Nc4ccc(N5CCN(CCO)CC5)c(F)c4F)nc23)c1]

The free base of CX-101 is known by the chemical name: N-(3-(2-((2,3-difluoro-4-(4-(2-hydroxyethyl)piperazin-1-yl)phenyl)amino)quinazolin-8-yl)phenyl)acrylamide. CX-101 is disclosed in WO 2015/027222. CX-101 is also known as RX-518.

HS-10296 (almonertinib; aumolertinib)

HS-10296 (almonertinib; aumolertinib) has the following chemical structure:

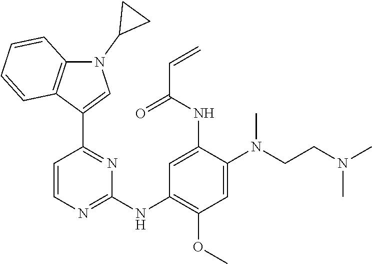 [SMILES: C=CC(=O)Nc1cc(Nc2nccc(-c3cn(C4CC4)c4ccccc34)n2)c(OC)cc1N(C)CCN(C)C]

The free base of HS-10296 is known by the chemical name: N-[5-[[4-(1-cyclopropylindol-3-yl)pyrimidin-2-yl]amino]-2-[2-(dimethylamino)ethyl-methyl-amino]-4-methoxy-phenyl]prop-2-enamide. HS-10296 is disclosed in WO 2016/054987.

In an aspect, the total daily dose of HS-10296 is about 110 mg.

BPI-7711 (rezivertinib)

BPI-7711 has the following chemical structure:

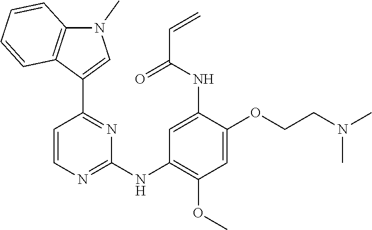 [SMILES: C=CC(=O)Nc1cc(Nc2nccc(-c3cn(C)c4ccccc34)n2)c(OC)cc1OCCN(C)C]

The free base of BPI-7711 is known by the chemical name: N-[2-[2-(dimethylamino)ethoxy]-4-methoxy-5-[[4-(1-methylindol-3-yl)pyrimidin-2-yl]amino]phenyl]prop-2-enamide. BPI-7711 is disclosed in WO 2016/94821.

In an aspect, the total daily dose of BPI-7711 is about 180 mg.

### Dacomitinib

Dacomitinib has the following chemical structure:

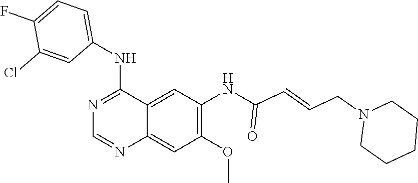 [SMILES: COc1cc2ncnc(Nc3ccc(F)c(Cl)c3)c2cc1NC(=O)/C=C/CN1CCCCC1]

The free form of dacomitinib is known by the chemical name: (2E)-N-{4-[(3-chloro-4-fluorophenyl)amino]-7-methoxyquinazolin-6-yl}-4-(piperidin-1-yl)but-2-enamide. Dacomitinib is described in WO 2005/107758. Dacomitinib is also known by the name PF-00299804.

Dacomitinib may be found in the form of dacomitinib monohydrate, i.e. (2E)-N-{4-[(3-chloro-4-fluorophenyl)amino]-7-methoxyquinazolin-6-yl}-4-(piperidin-1-yl)but-2-enamide monohydrate.

In an aspect, dacomitinib, or a pharmaceutically acceptable salt thereof, is administered once-daily. In a further aspect, dacomitinib monohydrate is administered once-daily.

In an aspect, the total daily dose of dacomitinib monohydrate is about 45 mg.

In an aspect, dacomitinib, or a pharmaceutically acceptable salt thereof, is in tablet form.

In an aspect, dacomitinib, or a pharmaceutically acceptable salt thereof, is administered in the form of a pharmaceutical composition comprising one or more pharmaceutically acceptable excipients. In a further aspect, the one or more pharmaceutically acceptable excipients comprise lactose monohydrate, microcrystalline cellulose, sodium starch glycolate and magnesium stearate.

### Icotinib

Icotinib has the following chemical structure:

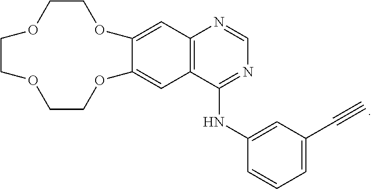 [SMILES: C#Cc1cccc(Nc2ncnc3cc4c(cc23)OCCOCCOCCO4)c1]

The free base of icotinib is known by the chemical name: N-(3-ethynylphenyl)-2,5,8,11-tetraoxa-15,17-diazatricyclo[10.8.0.0^{14′19}]icosa-1(12),13,15,17,19-pentaen-18-amine. Icotinib is disclosed in WO2013064128. Icotinib is also known as Conmana.

In instances, icotinib, or a pharmaceutically acceptable salt thereof, is administered three times daily. In further instances, icotinib hydrochloride is administered three times daily.

In instances, the total daily dose of icotinib hydrochloride is about 375 mg.

### Gefitinib

Gefitinib has the following chemical structure:

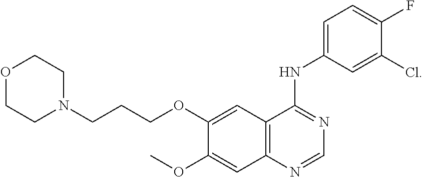 [SMILES: COc1cc2ncnc(Nc3ccc(F)c(Cl)c3)c2cc1OCCCN1CCOCC1]

The free base of gefitinib is known by the chemical name: N-(3-chloro-4-fluorophenyl)-7-methoxy-6-(3-morpholin-4-ylpropoxy)quinazolin-4-amine. Gefitinib is disclosed in WO 1996/033980. Gefitinib is also known as IRESSA™.

In instances, gefitinib, or a pharmaceutically acceptable salt thereof, is administered once-daily. In further instances, gefitinib is administered once-daily.

In instances, the total daily dose of gefitinib is about 250 mg.

### Erlotinib

Erlotinib has the following chemical structure:

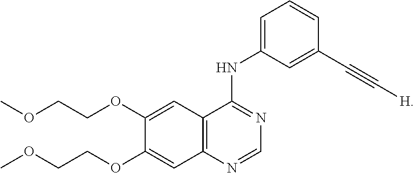

The free base of erlotinib is known by the chemical name: N-(3-ethynylphenyl)-6,7-bis(2-methoxyethoxy) quinazolin-4-amine. Erlotinib is disclosed in WO 1996/030347. Erlotinib is also known as TARCEVA™.

In instances, erlotinib, or a pharmaceutically acceptable salt thereof, is administered once-daily. In further instances, erlotinib is administered once-daily.

In instances, the total daily dose of erlotinib is about 150 mg.

In instances, the total daily dose of erlotinib is about 100 mg.

### Combination Treatment

As explained above, co-expression of EGFR and cMET is associated with many cancer types and antibody molecules that target both molecules and especially conjugates comprising such antibody molecules provide an opportunity for broad clinical benefit across multiple indications. The combination of EGFR TKI and antibody molecule as described herein are thus expected to be useful for therapeutic applications, in particular in the treatment of cancer.

An EGFR TKI and/or antibody molecule as described herein may be used in a method of treatment of the human or animal body. Related aspects of the disclosure provide;
(i) an EGFR TKI described herein for use in a method of treatment of a cancer, wherein in the method the EGFR TKI is administered in combination with an antibody molecule described herein,
(ii) an antibody molecule described herein for use in a method of treatment of a cancer, wherein in the method the antibody molecule is administered in combination with an EGFR TKI described herein,
(iii) use of an EGFR TKI described herein in the manufacture of a medicament for the treatment of cancer, wherein in the treatment the EGFR TKI is administered in combination with an antibody molecule described herein;
(iv) use of an antibody molecule described herein in the manufacture of a medicament for the treatment of cancer, wherein in the treatment the antibody molecule is administered in combination with an EGFR TKI described herein;
(v) a method of treating a cancer in an individual, wherein the method comprises administering to the individual a therapeutically effective amount of an antibody molecule as described herein in combination with a therapeutically effective amount of an EGFR TKI as described herein;
(vi) a method of treating a cancer in an individual, wherein the method comprises administering to the individual a first amount of an EGFR TKI, and a second amount of an anti-EGFR/cMET antibody molecule, where the first amount and the second amount together comprise a therapeutically effective amount.

Also provided herein is a pharmaceutical combination of an EGFR/cMET antibody molecule described herein and an EGFR TKI described herein. A pharmaceutical combination refers to a composition comprising a therapeutically effective amount of an EGFR/cMET antibody molecule described herein and a therapeutically effective amount of an EGFR TKI described herein and one or more pharmaceutically acceptable carriers, where each active ingredient is intended to be given to the patient in combination.

As used herein a “combination treatment” refers to the administration of both i) an antibody molecule described herein (which, as described herein may be conjugated to a drug); and ii) a EGFR TKI described herein to an individual.

The individual may be a patient. In some instances, the patient is a human patient.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

### Administration

The antibody molecules, conjugates and EGFR TKIs will usually be administered in the form of a pharmaceutical composition, which may comprise at least one component in addition to the active agent.

Pharmaceutical compositions may comprise, in addition to the antibody molecule, conjugate or EGFR TKI, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. Appropriate doses of EGFR TKIs are known in the art, which exemplary doses described above in the section on EGFR TKIs. Appropriate doses of antibody molecules are well known in the art (Ledermann, 1991; and Bagshawe, 1991). Specific dosages indicated herein, or in the Physician's Desk Reference (2003) as appropriate for an antibody molecule being administered, may be used. A therapeutically effective amount or suitable dose of an antibody molecule can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antibody molecule. The antibody molecule may be for example administered daily, once a week, once every two weeks or once every month. In some instances, the EGFR TKI is administered as a first amount and the anti-EGFR/cMET antibody molecule administered as a second amount, where the first amount and second amount together comprise a therapeutically effective amount.

The EGFR TKI may be administered to the individual concurrently with, sequentially to, or separately from the administration of the antibody molecule. Where the EGFR TKI is administered concurrently with the antibody molecule, the antibody molecule and EGFR TKI may be administered to the individual as a combined preparation.

The combination treatment (i.e. antibody molecule and EGFR TKI) may be administered in combination with a third treatment, said third treatment administered to the individual concurrently with, sequentially to, or separately from the combination treatment. The third treatment may comprise chemotherapy, radiotherapy, another (different) antibody molecule, or another (different) EGFR TKI.

### Cancer

The cancer to be treated using the combination treatment as described herein may be selected from the group consisting of: lung cancer (such as Non-Small Cell Lung Cancer (NSCLC)), pancreatic cancer, breast cancer, colorectal cancer, kidney cancer, gastric cancer, head and neck cancer, ovarian cancer or glioblastoma.

The cancer may be a cancer that expresses both EGFR and cMET. Cells of the cancer may express EGFR and cMET at the cell surface. In one instance, the tumour may have been determined to co-express EGFR and cMET. Methods for determining the expression of a target are known in the art and include, for example, immunohistochemistry.

In some instances, the cancer to be treated using the combination treatment described herein is selected from the group consisting of: lung cancer (such as Non-Small Cell Lung Cancer (NSCLC)), pancreatic cancer, colon cancer, colorectal cancer and squamous cell carcinoma of head and neck (SCCHN or SQHN). In one instance, the cancer to be treated in non-small cell lung cancer (NSCLC). In one instance, the cancer is squamous cell carcinoma of head and neck (SCCHN).

In some cases, the cancer is a wild-type EGFR cancer, an EGFR mutant cancer, a wild-type cMET cancer, or a cMET mutant cancer. Methods of detecting EGFR and cMET mutant cancers are well known.

In some instances, the cancer to be treated is an EGFR mutant cancer (also termed “EGFR mutation-positive”), for example an EGFR mutant NSCLC. Exemplary EGFR mutations, such as EGFR activating mutations, that may be associated with cancer include point mutations, deletion mutations, insertion mutations, inversions or gene amplifications that lead to an increase in at least one biological activity of EGFR, such as elevated tyrosine kinase activity, formation of receptor homodimers and heterodimers, enhanced ligand binding etc. Mutations can be located in any portion of an EGFR gene or regulatory region associated with an EGFR gene and include mutations in exon 18, 19, 20 or 21. In some instances, the EGFR mutant cancer is a cancer with a L858R mutation, one or more deletions in exon 19, or one or more insertions in exon 20, a T790M mutation or a combination thereof in the EGFR gene.

In NSCLC, specific mutations in the EGFR gene are associated with high response rates to EGFR TKIs. The single point mutation leucine-858 to arginine (L858R) in exon 21 and variable deletions of at least three amino acid residues in exon 19 are together often referred to as ‘classical’ EGFR activating mutations and represent the vast majority (85-90%) of all observed EGFR kinase domain mutations in NSCLC (Vyse and Huang, 2019). Examples of reported EGFR exon 19 deletions include delE746-A750, delE746-T751, delL747-E749, delL747-P753, delL747-T751. In some instances, the EGFR mutant cancer is a cancer (e.g. an EGFR mutation-positive NSCLC) with a L858R mutation and/or one or more deletions in exon 19 in the EGFR gene. In some instances, the EGFR mutant cancer is a cancer (e.g. an EGFR mutation-positive NSCLC) with one or more deletions in exon 19 and/or a L858R mutation in the EGFR gene. In some instances, the EGFR mutant cancer is a cancer (e.g. an EGFR mutation-positive NSCLC) with a L858R mutation and one or more deletions in exon 19 in the EGFR gene. In some instances, the EGFR mutant cancer is a cancer (e.g. an EGFR mutation-positive NSCLC) with one or more deletions in exon 19 in the EGFR gene. In some instances, the EGFR mutant cancer is a cancer (e.g. an EGFR mutation-positive NSCLC) with a L858R mutation in the EGFR gene.

Although the majority of NSCLC patients with EGFR mutations initially respond to EGFR TKI therapy, virtually all patients treated with first-generation EGFR TKIs acquire resistance after a progression-free period of approximately 10 months. A secondary point mutation that substitutes methionine for threonine at amino acid position 790 (T790M) is a molecular mechanism that produces a drug-resistant variant of the targeted kinase. The T790M mutation is present in about half of the lung cancer patients with acquired resistance to first- and second-generation EGFR TKIs, and reported to act by increasing the affinity of the receptor to adenosine triphosphate, relative to its affinity to TKIs (Suda, 2009).

Not all activating EGFR mutations are inherently sensitive to EGFR inhibitors. In-frame base pair insertions in exon 20 also result in constitutive activation of EGFR, but unlike the classical activating EGFR mutations, EGFR exon 20 insertions are associated with de novo resistance to current clinically available EGFR inhibitors. Low response rates of between 3-8% for erlotinib, gefitinib and the second generation EGFR inhibitor afatinib have been reported in EGFR exon 20 insertion mutant NSCLC patients and thus, effective treatment options are limited (Vyse and Huang, 2019). Examples of reported EGFR exon 20 insertions include D761-E762 insX, A764-Y764 insX, Y764-V765 insX, V765-M766 insX, A767-S768 insX, S768-V769 insX, V769-D770 insX, D770-N771 insX, N771-P772 insX, P772-H773 insX, H773-V774 insX, V774-C₇₇₅ insX, wherein insX indicates an in-frame insertion of between 1-7 amino acids.

As demonstrated herein, a combination treatment involving the anti-EGFR/cMET conjugate described herein and osimertinib (a third generation EGFR TKI) showed effective tumour inhibition across a range of different mutant EGFR cancer models, including those containing the L858R mutation, an exon 20 insertion, and an exon 19 deletion with a T790M mutation. Hence, it is expected that the combination treatment described herein will be effective at treating a range of different EGFR mutant cancers in humans.

In some instances, the patient being treated has previously been treated with a prior anti-cancer therapy, such as a prior EGFR TKI. In some cases, the human patient's disease has progressed during or after previous EGFR TKI treatment, i.e. the patient has acquired resistance or is resistant to treatment to the previous EGFR TKI treatment. In some cases, the patient being treated is resistant to or has acquired resistance to treatment with erlotinib, gefitinib, lapatinib, vandetanib, afatinib, osimertinib, poziotinib, criotinib, cabozantinib, capmatinib, axitinib, lenvatinib, nintedanib, regorafenib, pazopanib, sorafenib and/or sunitinib. In some cases, human patient's disease has progressed during or after previous treatment with a different EGFR TKI, e.g. the cancer being treated is classed as an osimertinib resistant cancer. As described above, mutations associated with EGFR TKI resistance include the T790M mutation and insertions in exon 20. In other instances, the patient being treated is an EGFR TKI-naïve human patient (i.e. they haven't previously been treated with an EGFR TKI).

### Therapeutic Effect

In the context of cancer, treatment may include inhibiting cancer growth, including complete cancer remission, and/or inhibiting cancer metastasis, as well as inhibiting cancer recurrence. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of anti-cancer immune cells or other anti-cancer immune responses, and a decrease in levels of tumour-specific antigens. Activating or enhancing immune responses to cancerous tumours in an individual may improve the capacity of the individual to resist cancer growth, in particular growth of a cancer already present the subject and/or decrease the propensity for cancer growth in the individual.

In some instances, the combination treatment described herein are capable of inhibiting the development or progression of a cancer.

The ability of a given combination treatment to inhibit the development or progression of a cancer can be analysed e.g. using an in vivo model. For example, the in vivo model may involve measuring tumour growth in a patient derived xenograft (PDX) model. Further details of this exemplary method is described in the examples.

Inhibition of the development of a cancer may be inferred by observation of slower tumour growth or a decrease in tumour size following administration of the antibody molecule, for example by measuring the tumour growth inhibition (% TGI). % TGI can be measured by comparing the size of the tumour measured at day 0 with the size of the tumour measured at the end of the study time for those subjects administered with the antibody molecule, and comparing this to the tumour growth over the same time period for subjects administered with a control antibody molecule. In this way, % TGI can be defined as Percent tumour growth versus Day 0 between treatment (TX) and control (C) groups, according to the formula: % TGI=(1−(TX_{final}−TXᵢₙᵢₜᵢₐₗ)/(C_{final}−Cᵢₙᵢₜᵢₐₗ))×100.

In some instances, the combination treatment described herein has a % TGI of at least 50%, at least 55%, at least 60%, at least 65%, at least 70%, at least 75%, at least 80%, at least 85%, at least 90%, at least 95%, at least 96%, at least 97%, at least 98%, or at least 99%.

Inhibition of the development of a cancer may be inferred by observation of a delayed or prevented onset of, and/or reduced severity of, symptoms of the cancer in response to treatment with the antibody molecule. Inhibition of the progression of a cancer may be inferred by observation of delayed, prevented and/or reduced invasion and/or metastasis in response to treatment with the antibody molecule.

The combination treatment described herein may be capable of inhibiting the development or progression of a cancer to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the development/progression of the cancer in the absence of treatment (or treatment with an appropriate control). In some instances, the combination treatment described herein is capable of inhibiting the development or progression of a cancer to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of development/progression of the cancer in the absence of treatment (or treatment with an appropriate control).

In some instances, the combination treatment described herein is capable of inhibiting the development or progression of a cancer to a greater extent than the use of the single agent (i.e. antibody molecule or EGFR TKI) administered individually. The combination treatment described herein may be capable of inhibiting the development or progression of a cancer to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the development/progression of the cancer treated with the antibody molecule, conjugate or EGFR TKI alone. In some instances, the combination treatment described herein is capable of inhibiting the development or progression of a cancer to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of development/progression of the cancer treated with the antibody molecule, conjugate or EGFR TKI alone. In some instances, the combination treatment described herein exerts a synergistic effect on inhibiting the development or progression of a cancer.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary instances described above, many equivalent modifications and variations will be apparent to those skilled in the art when given this disclosure. Accordingly, the exemplary instances of the disclosure set forth above are considered to be illustrative and not limiting. Various changes to the described instances may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another instance includes from the one particular value and/or to the other particular value. Similarly, when values are expressed as approximations, by the use of the antecedent “about,” it will be understood that the particular value forms another instance. The term “about” in relation to a numerical value is optional and means for example +/−10%.

### EXAMPLES

### Example 1—RAA22/B09 DuetMab Design and Construction

This example describes the creation of bispecific antibody molecules that are capable of binding both EGFR and cMET.

### 1.1 Isolation and Identification of Anti-cMET Antibody 0021U3-B09

cMET-specific scFv antibodies were isolated from a large naïve human scFv phage display library in a series of repeated panning selection cycles on recombinant mammalian expressed biotinylated monomeric human cMET (MedImmune) essentially as described (Vaughan, 1996). ScFvs from the round 2 of the selection output were expressed in the bacterial periplasm and screened for their ability to inhibit the binding of the human cMET receptor with the HGF ligand in a HGF:cMET HTRF® (Homogeneous Time-Resolved Fluorescence) ligand receptor inhibitory binding assay. Top hits exhibiting strong inhibitory effect were selected and subjected to DNA sequencing. Unique genes were then converted to human immunoglobulin G2 (IgG2) antibodies and produced in mammalian cells essentially as described (Persic; 1997). The purified antibodies were then ranked based on their inhibitory effect in the HGF:cMET HTRF® binding assay. The most potent antibody, 0021U3-B09, was selected for further characterization.

### 1.2 Optimization of Anti-cMET Antibody 0021U3-B09.

To minimize potential immunogenicity, non-Vernier framework residues (Foote and Winter 1992) in the variable framework regions of 0021U3-B09 were targeted specifically and altered to match the closest human germline sequence. In the VH region, seven amino acid residues were mutated to match the reference human germline sequence IGHV1-8*01. In the VL region, three residues were mutated to match the reference human germline sequence IGKV1-5*03. All residues in VH and VL regions were successfully changed to the germline residues without loss of activity. 0021U3-B09 was affinity optimized using a hybridization-based mutagenesis method essentially as described (Kunkel 1985). A large scFv library derived from 0021U3-B09 sequence was created by oligonucleotide-directed mutagenesis of the VH complementarity determining regions 3 (CDR3) using standard molecular biology techniques. The library was subjected to affinity-based solution phase selections to select variants with a higher affinity to human and cynomolgus cMET antigens. Crude scFv-containing periplasmic extracts from the CDR-targeted selection outputs were screened for improved inhibitory activity in the HGF:cMET HTRF® binding assay. Variants exhibiting significantly improved inhibitory effect compared to parent 0021U3-B09, were subjected to DNA sequencing and unique genes were converted to human IgG2. The purified antibodies were then ranked based on their inhibitory effect. The most potent antibody, B09-57, was selected for further characterization.

### 1.3 Isolation and Identification of Anti-EGFR Antibody Tdev-0004.

EGFR-specific scFv antibodies were isolated from a large naïve human scFv phage display library in a series of repeated panning selection cycles on recombinant mammalian expressed biotinylated monomeric human EGFR (MedImmune) essentially as described (Vaughan, 1996). ScFv-displaying phage from the round 3 of the selection output were screened for their binding to human and cynomolgus EGFR in ELISA. Top hits showing cross reactivity were selected and subjected to DNA sequencing. Unique genes were then converted to human immunoglobulin G1 (IgG1) antibodies and produced in mammalian cells essentially as described (Persic, 1997). The purified antibodies were then ranked based on their binding to the EGFR-expressing cell line, A431, by flow cytometry. Antibody Tdev-0004 exhibiting specific cell binding was selected for further characterization.

### 1.4 Optimization of Anti-EGFR Antibody Tdev-0004.

Variant RAA22 and QD6, were derived by optimizing the anti-EGFR Tdev-0004 mAb. The VL frameworks of Tdev-0004 had 100% match to the reference human germline sequence IGLV2-11*01/IGLJ2 (see https://www.ncbi.nlm.nih.gov/projects/igblast/Idlink.cgi?seqname=IGLV2-11*01&taxid=9606&dbname=IG_DB %2Fimgt.Homo_sapiens.V.forfp), however, the VH frameworks had 79% homology with the closest human germline IGHV1-69*01/JH4 (see https://www.ncbi.nlm.nih.gov/projects/igblast/Idlink.cgi?seqname=IGHV1-69*01&taxid=9606&dbname=IG_DB %2Fimgt.Homo_sapiens.V.forfp). To minimize potential immunogenicity, the VH region was initially fully germlined by mutating all 13 non-germline framework residues. Upon germlining, the binding of the fully germlined variant to cynomolgus EGFR was significantly impaired. To restore the binding to cynomolgus EGFR, four non-germline residues; K68, 173, R⁷⁶ and T78 were selectively back mutated. Amino acid residues are numbered by Kabat numbering system (Kabat and Wu 1991). The resulting, partially germlined variant, named H4, was used as a template sequence for the affinity optimization. Variant H4 was affinity optimized by parsimonious mutagenesis of all six CDRs using a QuikChange Lightning Multi Site-Directed Mutagenesis Kit (Agilent), according to the manufacturer's instructions. Single amino acid mutagenized VH and VL libraries were expressed in bacteria as Fab fragments and screened for improved binding to human and cynomolgus EGFR in ELISA. Variants exhibiting improved binding compared to parent H4 were subjected to DNA sequencing and unique genes were converted to human IgG1. Variant RAA22 was identified with a single mutation in CDRH3. To further improve the affinity, individual positive mutations were combined to create a combinatorial library that was screened for variants with enhanced binding to human and cynomolgus EGFR. Variant QD6 was identified with four combined mutations in CDRL2, CDRL3 and CDRH3.

1.5 Generation of Monovalent Bispecific Anti-EGFR cMET DuetMab Antibodies.

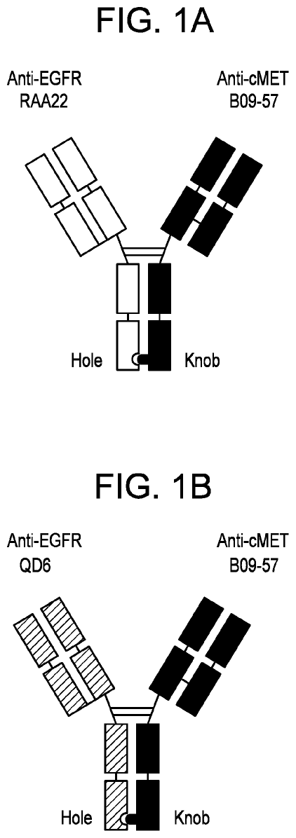

The variable domains of the anti-cMET mAb B09-57 and anti-EGFR mAbs RAA22 and QD6 were utilized for the construction of monovalent bispecific anti-EGFR/cMET antibodies on the backbone of the DuetMab platform (Mazor, 2015). Specifically, the VH gene of the anti-cMET B09-57 was inserted into a human gamma-1 constant heavy chain carrying the “Knob” mutation (T366W) and the alternative interchain cysteine mutations (F126C and C219V). The VL gene of B09-57 was inserted in frame into a human Kappa constant domain carrying the corresponding alternative interchain cysteine mutations (S121C and C214V) designed to pair with the “Knob” heavy chain. Similarly, the VH genes of the anti-EGFR RAA22 and affinity optimized QD6 were inserted into a human gamma-1 constant heavy chain carrying the “Hole” mutations (T366S, L368A, and Y407V), while the VL genes of RAA22 and B09-57 were inserted in frame into a human Lambda constant domain designed to pair with the “Hole” heavy chain. In addition, two residues in the CH3 domains of “Knob” and “Hole” heavy chains were mutated to cysteine (S354C in “Knob” and Y349C in “Hole”) to form a stabilizing disulfide bridge. The Fc domain was further engineered to carry a Cysteine insertion after Serine 239 (C239i/“Maia”) designed to enable site-specific conjugation of maleimide-bearing cytotoxic drugs (Dimasi, 2017). Amino acid residues are numbered by Kabat numbering system (Kabat and Wu 1991). The assembled monovalent bispecific anti-EGFR/cMET DuetMab antibodies were designated as RAA22/B09-57 and QD6/B09-57 (FIG. 1). DuetMab antibodies were produced from mammalian cells as previously described (Mazor, 2017).

The amino acid sequences of the heavy and light chains of the DuetMabs produced according to this example are provided in the following table:

| | RAA22/B09-57 | QD6/B09-57 |
|---|---|---|
| EGFR heavy chain | 56 | 53 |
| cMET heavy chain | 50 | 50 |
| EGFR light chain | 58 | 55 |
| c-Met light chain | 52 | 52 |

### Example 2—Biochemical and Biophysical Properties

This example tests various biochemical and biophysical properties of the RAA22, QD6 and B09-57 monoclonal antibodies and RAA22/B09-57 and QD6/B09-57 bispecific antibodies molecules, including their binding affinity to EGFR and c-Met, respectively and their ability to bind both antigens simultaneously.

2.1 Binding Affinity of DuetMabs and Parental mAbs for EGFR and cMET.

The kinetic rate constants (kon and koff), and equilibrium dissociation constants (KD) of EGFR-cMET DuetMAbs for recombinant human, cynomolgus monkey, and murine EGFR and cMET antigens were determined at 25° C. by SPR using an antibody capture assay on a BIAcore T200 instrument (GE Healthcare, Pittsburgh, PA). Mouse anti-human IgG was immobilized on a CM4 sensor chip with a final surface density of ˜2000 resonance units (RUs). A reference flow cell surface was also prepared on this sensor chip using identical immobilization protocol. Test and control article antibodies were prepared at 5-20 nM in instrument buffer (HBS-EP buffer; 0.01M HEPES, pH 7.4, 0.15M NaCl, 3 mM EDTA, and 0.005% P-20), along with 3-fold serial dilutions of purified EGFR (0.27-200 nM human, 0.4-900 nM cyno, and 4-1000 nM murine) or cMET proteins (0.27 to 66 nM human and 0.27-22 nM cyno) in instrument buffer. A sequential approach was utilized for kinetic measurements. Antibodies were first injected over the capture surface, at a flow rate of 10 μL/minute. Once the binding of the captured antibody stabilized, a single concentration of the analyte was injected over both capture and reference surfaces, at a flow rate of 75 μL/minute. The resulting binding response curves yielded the association phase data. Following the injection of analyte, the flow was then switched back to instrument buffer for 15 minutes to permit the collection of dissociation phase data, followed by a 1-minute pulse of 10 mM glycine, pH 1.5, to regenerate the antibody-captured surface on the chip. Binding responses against test and control article antibodies were recorded from duplicate injections of each concentration of analyte. In addition, several buffer injections were interspersed throughout the injection series. Select buffer injections were used along with the reference cell responses to correct the raw data sets for injection artifacts and/or nonspecific binding interactions, commonly referred to as “double referencing”. Corrected binding data were globally fit to a 1:1 binding model (Biacore T200 Evaluation software 2.0, GE Healthcare, Pittsburgh, PA). The calculated kinetic parameters (kₒₙ and k_{off}) and K_{D} determined as k_{off}/kₒₙ are shown in Table 1.

TABLE 1
Kinetics of DuetMabs and parental IgGs to EGFR and cMET Antigens

| Antibody | Antigen | Kₒₙ (M⁻¹ s⁻¹) | K_{off} (s⁻¹) | K_{D} (nM) |
|---|---|---|---|---|
| RAA22 IgG | Human EGFR | 4.41 × 10⁴ | 2.06 × 10⁻³ | 46.6 |
| | Cynomolgus monkey | 3.54 × 10⁴ | 6.01 × 10⁻³ | 169.8 |
| | EGFR | | | |
| | Mouse EGFR | 4.03 × 10⁴ | 1.90 × 10⁻² | 488.0 |
| | Human cMET | | | |
| | Cynomolgus monkey | No binding detected at 200 nM | | |
| | cMET | | | |
| | Mouse cMET | | | |
| QD6 IgG | Human EGFR | 1.48 × 10⁵ | 2.92 × 10⁻⁴ | 2.0 |
| | Cynomolgus monkey | 1.14 × 10⁵ | 2.88 × 10⁻⁴ | 2.5 |
| | EGFR | | | |
| | Mouse EGFR | 1.67 × 10⁵ | 7.35 × 10⁻⁴ | 4.4 |
| | Human cMET | | | |
| | Cynomolgus monkey | ND | | |
| | cMET | | | |
| | Mouse cMET | | | |
| B09-57 IgG | Human EGFR | | | |
| | Cynomolgus monkey | No binding detected at 200 nM | | |
| | EGFR | | | |
| | Mouse EGFR | | | |
| | Human cMET | 5.63 × 10⁵ | 8.82 × 10⁻⁴ | 1.6 |
| | Cynomolgus monkey | 1.04 × 10⁶ | 1.94 × 10⁻³ | 1.9 |
| | cMET | | | |
| | Mouse cMET | No binding detected at 200 nM | | |
| RAA22/B09-57 | Human EGFR | 4.47 × 10⁴ | 2.01 × 10⁻³ | 45.0 |
| DuetMab | Cynomolgus monkey | 2.78 × 10⁴ | 5.48 × 10⁻³ | 197.0 |
| | EGFR | | | |
| | Mouse EGFR | 3.54 × 10⁴ | 2.06 × 10⁻² | 575.4 |
| | Human cMET | 4.43 × 10⁵ | 9.86 × 10⁻⁴ | 2.2 |
| | Cynomolgus monkey | 9.74 × 10⁵ | 2.12 × 10⁻³ | 2.2 |
| | cMET | | | |
| | Mouse cMET | ND | | |
| QD6/B09-57 | Human EGFR | 6.35 × 10⁴ | 3.74 × 10⁻⁴ | 5.9 |
| DuetMab | Cynomolgus monkey | 2.10 × 10⁵ | 5.85 × 10⁻⁴ | 2.8 |
| | EGFR | | | |
| | Mouse EGFR | 1.26 × 10⁵ | 7.80 × 10⁻⁴ | 6.2 |
| | Human cMET | 4.25 × 10⁵ | 6.96 × 10⁻⁴ | 1.6 |
| | Cynomolgus monkey | 9.58 × 10⁵ | 2.06 × 10⁻³ | 2.2 |
| | cMET | | | |

| | |
|---|---|
| Mouse cMET | ND |

^{a}ND: not determined.
Kinetic measurements to soluble monomeric forms of EGFR and cMET were performed using a BIACore instrument.
K_{D} were calculated as the ratio of k_{off}/kₒₙ from a non-linear fit of the data.

As can be seen from the above data, bispecific antibody molecule QD6/B09-57 binds human c-Met with a high affinity (˜2 nM kD) and human EGFR with a high affinity (˜6 nM kD), whilst bispecific antibody molecule RAA22/B09-57 binds human c-Met with a similarly high affinity (˜2 nM kD) but binds human EGFR with a reduced affinity (˜45 nM kD) in comparison to QD6/B09-57.

### 2.2 Concurrent Binding of DuetMabs to EGFR and MET

Concurrent binding studies to recombinant human EGFR and cMET proteins were measured by biolayer interferometry on an Octet384 instrument essentially as described (Mazor, 2015). Briefly, His-tagged cMET antigen at 5 μg/mL in assay buffer [PBS pH7.2, 3 mg/mL bovine serum albumin (BSA), 0.05% (v/v) Tween 20] was initially captured on NI-NTA biosensors. Following a washing step to remove any unbound protein, the respective loaded biosensors were subjected to successive association and dissociation interactions, first with 66 nM of the antibodies and then with the EGFR antigen at 500 nM. Association and dissociation curves were calculated from a non-linear fit of the data using the Octet384 software v.9.0. As shown in FIG. 2 the DuetMabs demonstrated simultaneous binding to both antigens while the parental anti-cMET IgG exhibited specific binding only to cMET and the two anti-EGFR IgGs exhibited no binding to the cMET loaded sensors.

2.3 EGFR and c-Met Specificity

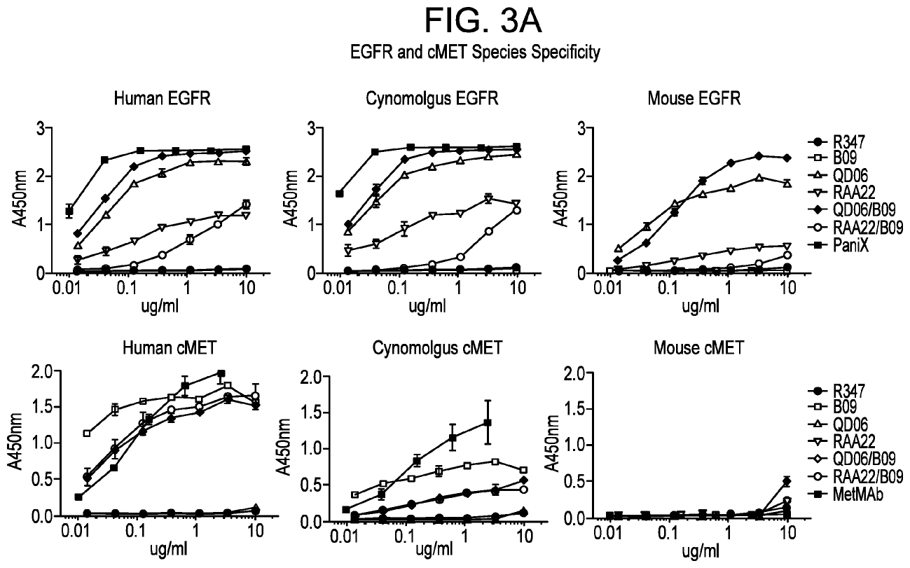

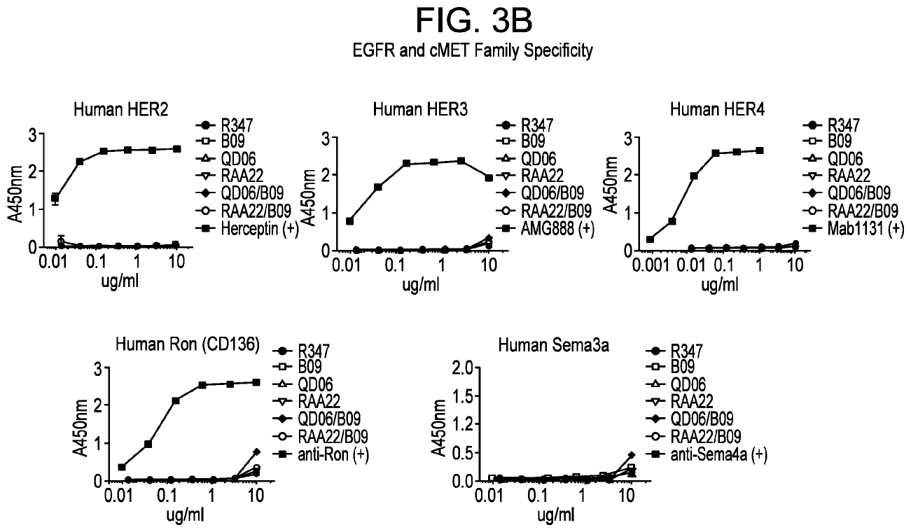

Specificity for EGFR and cMET species paralogs and closely related family members was determined by ELISA. Briefly, antigen solutions were prepared in PBS at 1 μg/mL and 50 microliters was coated onto half area ELISA assay plates. Plates were washed and blocked with 1% BSA in PBS containing 0.005% Tween-20 (PBS-T) for one hour at room temperature. The wells were washed 4 times in PBS-T. As set out in FIG. 3, the primary antibodies used where: R374 (a non-binding IgG1 isotype control antibody), B09 (anti-cMET antibody), QD6 (anti-EGFR antibody), RAA22 (anti-EGFR antibody), QD6/B09 (bispecific EGFR/c-MET DuetMAb), RAA22/B09 (bispecific EGFR/c-MET DuetMAb), PaniX (anti-EGFR antibody), MetMab (anti-cMET antibody) and Mab11311 (anti-HER4 antibody). Wells were incubated with 50 microliters of the indicated primary antibodies diluted in PBS-T in a 1:3 dilution series, starting at 10 μg/mL and ending at 0.002 μg/mL, except for the HER4 binding mAb control, MAB1131, for which the series started at 1 μg/mL. The wells were washed 4 times in PBS-T, then 50 μl of goat anti-human Fab HRP-labeled secondary antibody, diluted 1:5000 in PBS-T, was added to each well and incubated for one hour at room temperature. 50 microliters of TMB substrate solution was added to all wells and incubated at room temperature for 5-30 min, until intense signal was observed in the positive control wells. 50 microliters of TMB stop solution was added to all wells and the absorbance was read at 450 nm on a SpectraMax M5 microplate reader. Data were analyzed in the SoftMax Pro 5 software and plotted using GraphPad Prism 7 graphing software.

To determine the species cross reactivity, ELISA assays were carried out as described above. As shown in FIG. 3A, the high affinity EGFR IgG, QD6, as well as the monovalent bispecific EGFR/cMET DuetMAb, QD6/B09, bound to human, cynomolgus monkey, and mouse EGFR and gave robust signals in the ELISA assay. In contrast, the lowered affinity EGFR IgG, RAA22, bound more weakly to human, cynomolgus monkey, and mouse EGFR compared to QD6. Binding to mouse EGFR was weak, but detectable. The corresponding monovalent bispecific EGFR/cMET DuetMAb, RAA22/B09, showed still weaker binding to human and cynomolgus monkey EGFR relative to the bivalent parental IgG, RAA22, and nominal binding to mouse EGFR. The cMET IgG, B09, as well as all of the bispecific variants, showed comparable binding to human and cynomolgus monkey cMET. There was no detectable binding of any of the antibodies to mouse cMET. These results are consistent with the binding kinetics determined by surface plasmon resonance measurements on the BIAcore instrument (Table 1 above).

As shown in FIG. 3B none of the parental IgG or derivative bispecific antibodies showed appreciable binding to any of the EGFR HER family proteins, HER2, HER3, or HER4. Similarly, none of the antibodies showed significant binding to the cMET family members, Ron (CD136) or Semaphorin 3a.

These results demonstrate that the parental IgG's and the resulting bispecific antibodies bind specifically to their cognate targets, with no detectable binding to closely related family species.

### 2.4 Internalization of Bispecifpc Antibodies'

Internalization kinetics of AlexaFluor647 (AF647) primary-labeled DuetMabs: RAA22/B09 and QD6/B09 antibodies was assessed in vitro using the EGFR and c-MET expressing cell line, H1975. Each antibody was pre-bound to cells and antibody translocation from cell surface to cytoplasm was then monitored using live cell confocal fluorescence microscopy. Both antibodies localized primarily on the cell surface before (T=0) subjecting to internalization conditions and were translocated to the cytoplasm area after 1 hour (data not shown). Kinetic images were taken every 5 min over the time course of internalization and were processed using a quantitative algorithm to determine internalization kinetic constants and half-times. The internalization kinetics for QD6/B09 and RAA22/B09 were comparable, with the half-times of 37.5±10.6 min and 43.2±15.5, respectively.

To evaluate the mode of antibody internalization and to investigate contribution of each arm to overall internalization of the DuetMabs, we evaluated internalization of single-arm specific control antibody molecules, QD6/IgG and B09/IgG, against duet QD6/B09, and RAA22/IgG and B09/IgG against duet RAA22/B09 in H1975 cells, which express both EGFR and c-MET. Since only one arm is specific for the target receptor, the control antibody can only internalize via one receptor eliminating dual receptor targeting and cross-linking as mode of internalization.

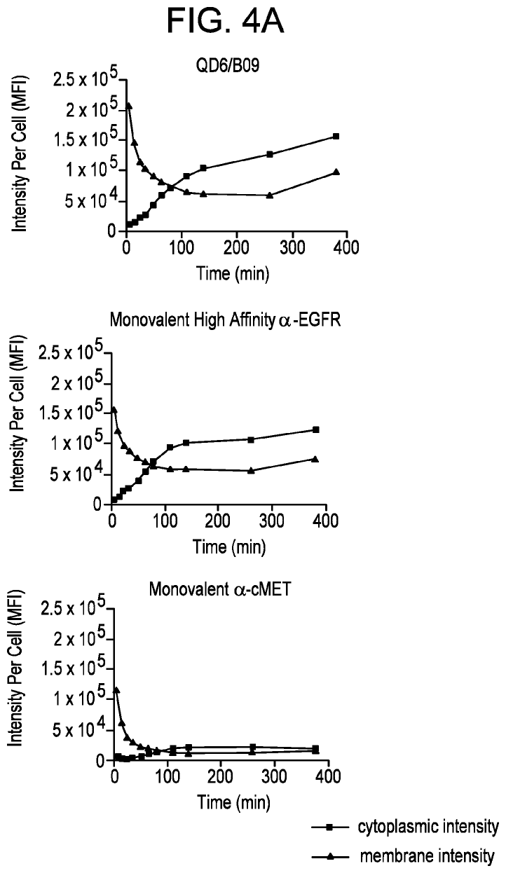

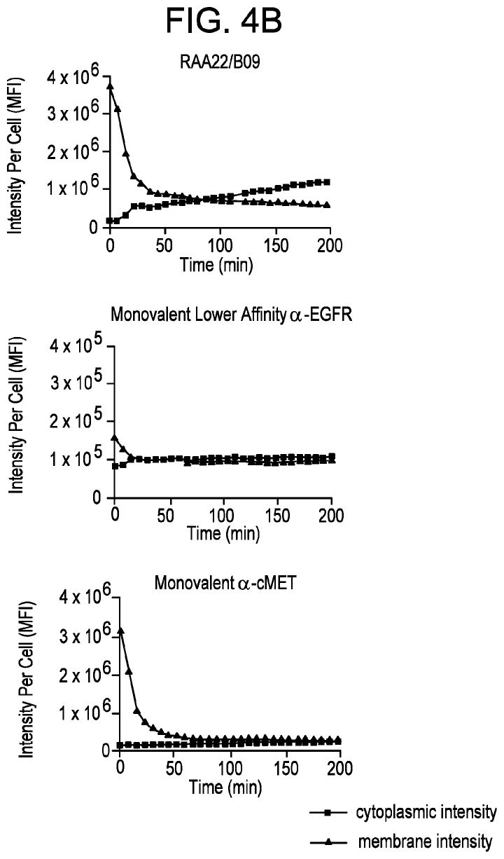

Internalization profiles of QD6/B09 (FIG. 4A) and RAA22/B09 (FIG. 4B) rendered very similar patterns of congruent decrease of membrane mAb-F1647 signals with respective increase mAb-AF647 signals in the cytoplasm, a typical profile for internalization. However, their single-arm constructs showed very different internalization profiles. Single-arm QD6/IgG had a near-identical internalization time course as the QD6/B09 DuetMab (FIG. 4A, left and middle), indicating that internalization of QD6/B09 duet was mostly driven by EGFR-arm of the molecule with minimal contribution of B09-arm. Indeed, the B09/IgG construct showed very small level of internalization (FIG. 4B, right). The rapid and extensive decrease of the membrane signal corresponded to a very moderate increase of the cytoplasm signal, likely due to extensive dissociation of pre-bound B09/IgG from c-MET receptor on the cell surface. The dissociation of the antibody subsequently resulted in modest internalization of B09/IgG. These results revealed that internalization of QD6/B09 duet was mostly driven by the EGFR-arm of the molecule with minimal contribution of the B09-arm.

In contrast, RAA22/B09 DuetMab showed internalization profiles very different when compared to its single-arm control antibodies. As seen in FIG. 4B, cytoplasmic intensity values were 10.98- and 4.70-fold higher for RAA22/B09 DuetMab than RAA22-IgG and B09-IgG, respectively. While inefficient internalization of B09/IgG maybe attributed to its pronounced dissociation, RAA22/IgG did undergo rapid internalization. However, due to the lower affinity of the EGFR-arm, the number of RAA22/IgG molecules were 10.98-fold less (based on fluorescent intensity) than for the RAA22-B09 DuetMab. The markedly increased amount of duet RAA22/B09 mAb entering the cytoplasm as opposed to the single-arm constructs demonstrated that both antibody arms must engage with target receptors to drive internalization. This finding shows that QD6/B09 and RAA22/B09 DuetMabs have different mechanisms of internalization, with QD6/B09 primarily driven by the EGFR-arm but RAA22/B09 requiring both EGFR and c-MET arms for engagement.

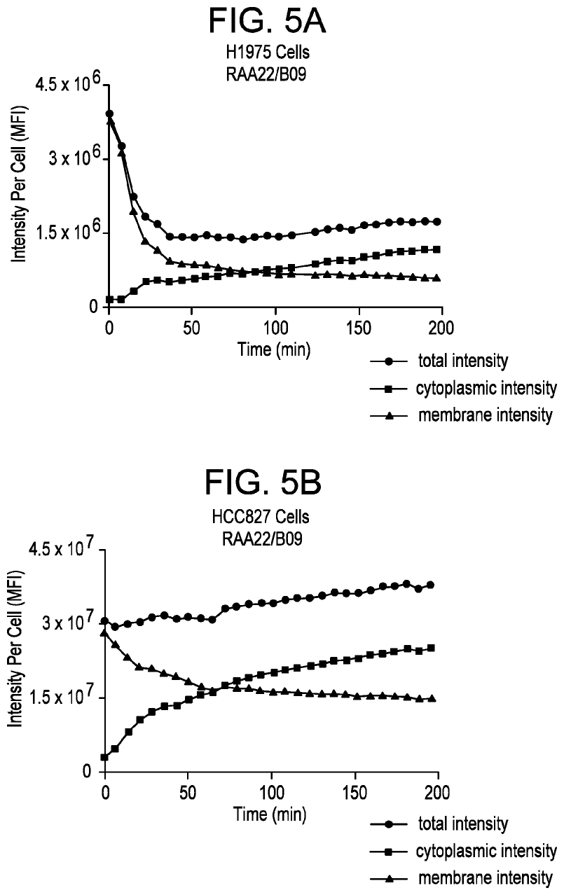

Since binding of both EGFR and c-MET arms to target receptor promoted RAA22/B09 receptor internalization in H1975 cells, we examined if the increased number of EGFR and c-MET receptors would affect internalization properties. The respective receptor levels determined by Western Blot were ˜33,000 for EGFR and ˜50,000 for c-MET in H1975 cells and ˜790,000 for EGFR and ˜523,000 for c-MET in HCC827 cells. Internalization profiles of RAA22/B09 in H1975 (medium receptor) and HCC827 (high receptor) cells shows markedly increased internalization in HCC827 cells (FIG. 5).

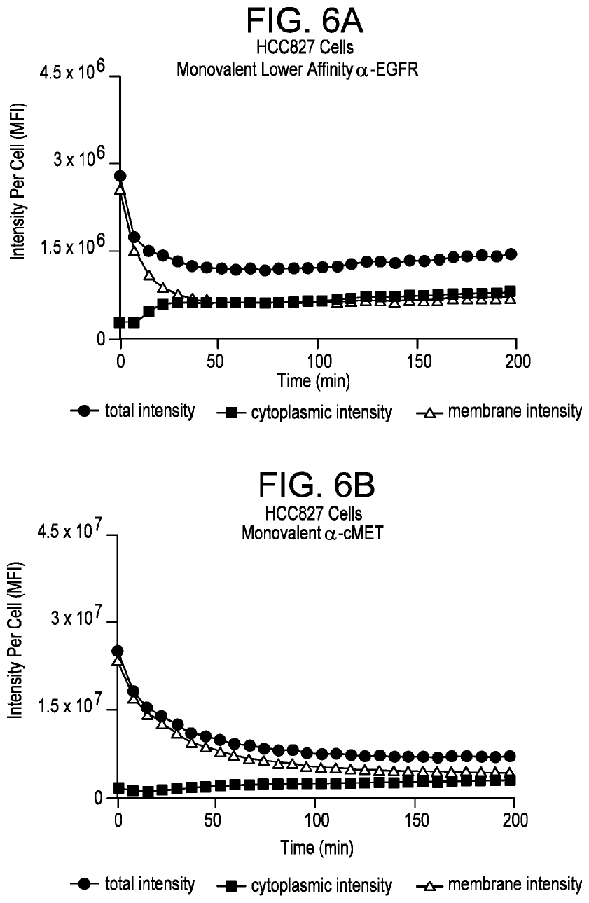

As expected in correspondence to the 23.9-fold and 10.4-fold respective increases in overall levels of EGFR and c-MET RAA22/B09, binding to HCC827 cells were on average 8.9-fold higher than H1975 (T=0, 3.1×10⁷ MFI versus 3.5×10⁶ MFI). Internalization levels (judged by peak cytoplasmic intensity) was 21.7-fold higher in HCC827 cells, suggesting that significantly higher concentrations of antibody enter the cytoplasm in cells expressing high levels of target receptors. Importantly, in addition to the markedly different intensities, the internalization profiles (membrane and cytoplasm signals over time) were also significantly distinct between HCC827 and H1975 cells. In high expressing HCC827 cells, the decrease of RAA22/B09-AF647 membrane signal corresponded to the reciprocal increase of RAA22-B09 cytoplasm signal with total RAA22/B09 signal maintained over the time course, indicating strong dual arm antibody interaction with both receptors and subsequent internalization. In H1975 cells, there was a concurrent decrease of total and membrane intensity, indicating that portions of pre-bound antibody could have dissociated from cell surface and failed to internalize inside the cell. Similar profiles indicative of dissociation were observed for internalization of RAA22/IgG and B09/IgG in HCC827 cells where single arm engagement did not render effective binding and was prone to dissociation (FIGS. 6A and B). This data suggested that mixed mode of receptor interaction (single arm and dual arm engagement) is present when RAA22/B09 is subjected to internalization in H1975 cells. Together, these data suggest that target cell receptor expression levels are an important determinant of the extent and efficiency of RAA22/B09 internalization.

### Example 3—In Vitro Potency of ADCs

In this example the in vitro potency of EGFR/cMET bispecific ADC's was measured in a panel of cancer cell lines.

### 3.1 Site Specific Conjugation

Conjugation of the tubulysin drug to the RAA22/B09 antibody molecule was carried out essentially as previously described in Thompson, 2016 and US20150291657A1.

### 3.2 Methods to Determine ADC Cytotoxic Activity

The ADC cytotoxic activity was tested in multiple cell lines as follows. Cells were plated at a density of 10,000 cells per well of 96-well plates in a volume of 100 μL in their recommended culture media supplemented with 10% fetal bovine serum. A 3× concentration of each dose of antibody to be tested was prepared by serial dilution of the antibody stock in culture medium. Fifty microliters of each test article was added to cells in triplicate such that the final antibody concentration ranged from 60 nM to 0.0009 nM. The treated cells were cultured for 72 hours at 37 degrees C. in a humidified incubator. The metabolic activity was determined using CellTiter-Glo Luminescent Viability Assay from Promega according to manufacturer's instructions. Data were plotted as percent metabolic activity relative to untreated control. IC₅₀ values were determined using logistic non-linear regression analysis between the maximal viability (untreated cells) and the maximal response (peak inhibition) with GraphPad Prism software.

### 3.3 Results—In Vitro ADC Activity in Cell Line Panel

The in vitro potency of EGFR/cMET bispecific ADC's was measured in a panel of cancer cell lines using the CellTiter-Glo Luminescent Viability Assay. As shown in Table 3, both the higher affinity QD6/B09-AZ1508 and lowered affinity RAA22/B09-AZ1508 exhibited broad activity across cell lines with a range of target expression levels.

TABLE 3
In Vitro Activity of EGFR-cMET DuetMAb ADC's

| | | | High Affinity ADC | | Lowered EGFR Affinity ADC | |
|---|---|---|---|---|---|---|
| | Relative | Relative | QD6/B09-AZ1508 | | RAA22/B09-AZ1508 | |
| | EGFR | cMET | | Max | | Max |
| Cell Line | density | density | IC50 (pM) | % Inhibition | IC50 (pM) | % Inhibition |
| HCC 827 | 1,513,899 | 236,933 | 96 | 79% | 132 | 55% |
| Parental | | | | | | |
| NCI H596 | 938,023 | 28,353 | 274 | 54% | 3,237 | 67% |
| HCC 827 | 760,040 | 523,298 | 33 | 66% | 168 | 59% |
| GR Pool | | | | | | |
| A549 | 88,385 | 16,483 | 118 | 45% | 667 | 36% |
| NCI | 63,820 | 20,658 | 81 | 80% | 200 | 81% |
| H1792 | | | | | | |
| NCI | 33,086 | 50,339 | 178 | 65% | 255 | 65% |
| H1975 | | | | | | |
| NCI H292 | 96,538 | 16,497 | 91 | 79% | 271 | 62% |
| A427 | 20,236 | 9,734 | 6,959 | 12% | 40,220 | 58% |
| NCI H358 | 15,065 | 16,429 | 168 | 29% | 3,017 | 30% |
| NCI H23 | 9,843 | 21,669 | >60,000 | ~5% | 8,096 | 46% |
| NCI H661 | ND - 9,906 | ND - 8,114 | >60,000 | 0 | >66,667 | 10% |
| (Ag | | | | | | |
| negative) | | | | | | |

Overall, the ADC with lowered affinity for EGFR showed comparable, though somewhat reduced, potency in a broad range of cell lines that co-express significant amounts of both EGFR and cMET. Generally, both ADC's showed reduced potency when one or the other target had a low relative receptor density at the cell surface of about 15,000 or less. This effect was more pronounced with the lowered affinity variant, which appeared more sensitive to lower levels of cMET.

### Example 4—Bispecific Engagement of the ADC

To further test the hypothesis that bispecific engagement of the lowered affinity EGFR-cMET antibody is required for optimal ADC delivery, we conducted in vitro experiments to determine the relative contribution of the individual antibody arms to the activity of the bispecific ADC. In the first experiment, we used an excess of unarmed parental antibodies to block either EGFR or cMET and then measured the activity of the bispecific EGFR-cMET ADC in an in vitro cytotoxicity assay. For this experiment, we used a cell line that expresses moderate levels of EGFR and cMET in roughly equal amounts. If the individual arms of the bispecific ADC function independently to deliver the ADC, blocking either target in this cell line would be expected to only modestly reduce the activity of the ADC, shifting the IC₅₀ by twofold or less, since the targets are present at similar levels. If, on the other hand, the ADC requires dual target engagement to effectively deliver the ADC into cells, blocking either target would be likely to have a greater impact on the activity of the bispecific ADC. In a related experiment, we compared the activity of the bispecific EGFR-cMET ADC to monovalent, monospecific control antibodies comprised of one binding arm to either EGFR or cMET and one non-binding isotype antibody control arm. Similarly, if each arm functions independently, the expected result would be that each monospecific control ADC would only be modestly less potent than the bispecific ADC, and the difference would be additive. Alternatively, if the two arms of the bispecific function synergistically, one would expect larger differences in activity of the bispecific ADC compared to the monospecific control antibodies.

### 4.1 Methods

The cytotoxic activity of ADC's were tested in the NCI-H1975 cell line as follows. Cells were plated at a density of 10,000 cells per well of 96-well plates in a volume of 50 μL for the blocking experiment and 100 μL for the monovalent ADC experiment in their recommended culture media supplemented with 10% fetal bovine serum. For the unarmed mAb blocking experiments, 50 μL of a 300 μg/mL solution of either EGFR IgG (RAA22) or cMET IgG (B09) was added to the wells and pre-incubated for one hour at 37 degrees C. in a humidified incubator. A 3× concentration of each dose of antibody to be tested was prepared by 4× serial dilution of the antibody stock in culture medium. Fifty microliters of either media alone, isotype control IgG ADC (R347-AZ1508), or EGFR-cMET ADC (RAA22/B09-AZ1508) was added to cells in triplicate such that the final antibody concentration ranged from 67 nM down to 0.0009 nM. For the monovalent ADC experiment, 50 μL of 3× stocks of either isotype control ADC (R347-AZ1508), monovalent EGFR ADC (RAA22/R347-AZ1508), monovalent anti-cMET ADC (B09/R347-AZ1508), equimolar combinations of the monovalent ADC's, or EGFR-cMET ADC (RAA22/B09-AZ1508) was added in triplicate in a 4× dilution series starting at 60 nM and ending at 0.009 nM. The treated cells were cultured for 72 hours at 37 degrees C. in a humidified incubator. The metabolic activity was determined using CellTiter-Glo Luminescent Viability Assay from Promega according to manufacturer's instructions. Data were plotted as percent metabolic activity relative to untreated control. IC₅₀ values were determined using logistic non-linear regression analysis between the maximal viability (untreated cells) and the maximal response (peak inhibition) with GraphPad Prism software.

### 4.2 Results and Conclusions—In Vitro Proof of Concept for Dual Targeting (mAb Blocking Experiment and Monovalent ADC)

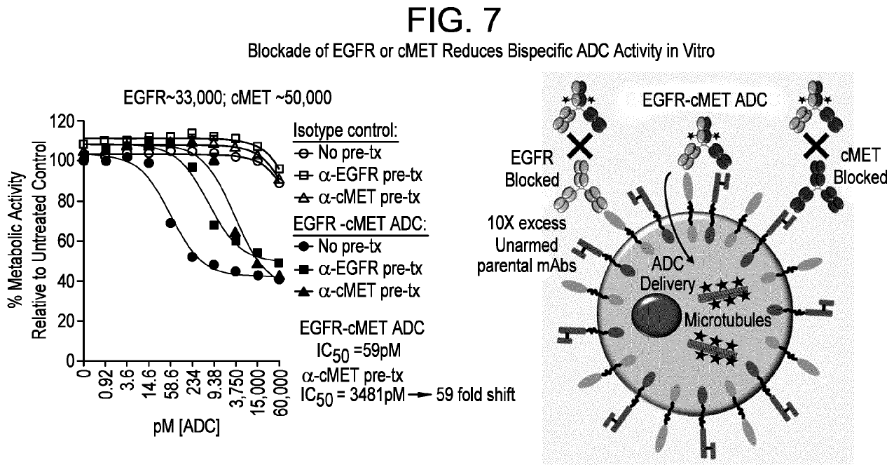

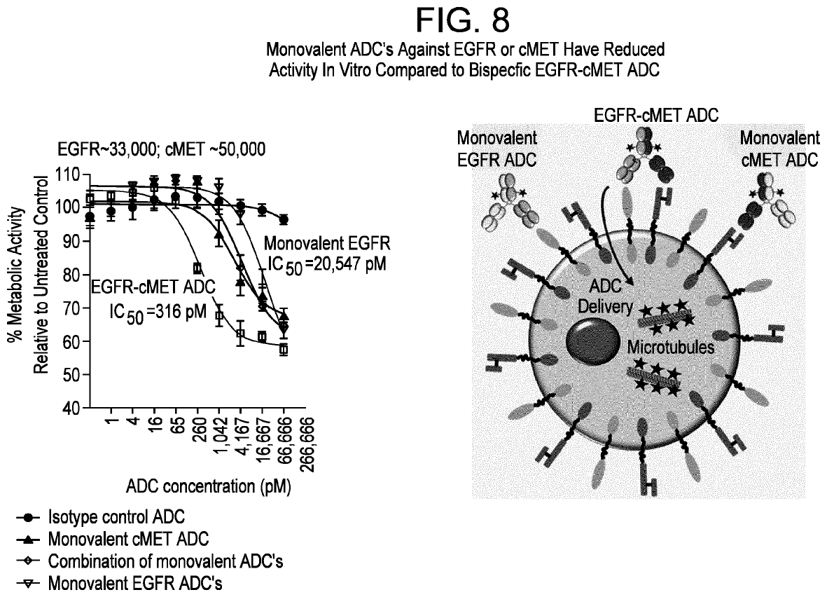

We conducted in vitro experiments to examine the relative contribution of the individual antibody arms to the cytotoxic activity of the bispecific ADC, as outlined above. As shown in the representative experiment in FIG. 7 pre-treatment of NCI H1975 cells with cMET IgG RAA22 resulted in a shift in IC₅₀ of the EGFR-cMET ADC (RAA22/B09-AZ1508) from approximately 60 μM to 3,480 μM, a difference of about 60 fold. Treatment with anti-cMET IgG B09 resulted in a shift in IC₅₀ to 680 μM, a difference of greater than 11 fold. Similarly, when NCI H1975 cells were treated with the monovalent monospecific EGFR ADC (RAA22/R347-AZ1508), the IC₅₀ was about 20,500 μM compared to 316 μM for the bispecific EGFR-cMET ADC, a difference of about 65 fold (FIG. 8). The IC₅₀ of the monovalent monospecific anti-cMET ADC (B09/R347-AZ1508) was 2,772 μM, a difference of about 13 fold compared to the bispecific antibody.

Collectively, these data suggest that efficient targeting of the EGFR-cMET ADC to tumor cells co-expressing both targets is largely driven by bispecific engagement of the ADC. Furthermore, these results show that the EGFR affinity reduced binding arm is insufficient to promote efficient ADC delivery in the absence of cMET binding, as demonstrated by the dramatic reduction of potency when the cMET arm is blocked by an unarmed antibody and by the weak cytotoxicity of the monovalent EGFR control ADC, RAA22/R347-AZ1508. Taken together, these results are consistent with the hypothesis that the reduced affinity of the EGFR binding arm of the bispecific ADC (RAA22/B09-AZ1508) will promote ADC delivery to EGFR and cMET co-expressing tumors, while exhibiting reduced cytotoxicity toward cells that primarily express only one of the targets. This effect is most prominent when only EGFR is available for engagement, which has implications for mitigating EGFR driven toxicities in normal organs, such as the skin, which expresses significant levels of EGFR but relatively little cMET.

### Example 5—ADC In Vivo Pharmacology in Patient Derived Xenograft (PDX) Models

Patient derived xenograft (PDX) models of human cancer have become a well-established alternative to tumor cell line based tumor xenografts. PDX models are established from a patient's primary tumor tissue implanted directly into immunodeficient mice to yield in vivo propagated tumors in the mouse. The tumors thus derived are subsequently propagated in additional mice, without culturing in vitro, to establish a bank of low passage PDX tumor tissue which can be used to implant study mice. One key feature of PDX models is that they largely maintain the histological and genomic heterogeneity and preserve the gene expression profile of the corresponding original patient tumor. Compared to tumor cell line based xenograft models, which use clonal populations of tumor cells that have been adapted to growth in vitro, the characteristics of PDX models are intended to more accurately replicate the features of real human tumors, thus improving the predictive value of pre-clinical mouse models. Indeed, numerous studies have shown that the response and resistance profiles of PDX models to standard of care treatments closely correlate with clinical data in human subjects with a given tumor profile.

Despite the improvements that PDX models afford, there are limitations to standard in vivo pharmacology study designs, even when applied to PDX models. For each tumor model, a typical study tests a drug treatment at multiple dose levels, along with one or more positive or negative control compounds, with sufficient mice per treatment group to support intra-model statistics. The relatively large number of mice used for such a study design and the higher cost of PDX models can limit the number of tumor models that one can practically test for a given compound. An alternate/complementary approach to the traditional study design is the mouse PDX trial, a population based approach styled after human clinical trial design. In this approach, for each compound, a single mouse is typically treated at a single dose level established from prior dose range finding studies, with an optional treatment control group for each model. Due to the small number of mice required for each model, many PDX models can be tested, each representing a unique human tumor. Instead of relying on intra-model statistics, responses are evaluated across the entire population of tumor models tested. This approach can provide a more accurate estimate of response rate across a diverse range of target expression, molecular phenotypes, tumor subtypes, or other clinically relevant features of interest. Furthermore, the large number of unique models that one can test in a PDX trial can enable more meaningful exploratory genomics, transcriptomics, or expression profiling studies to begin the early search for correlates of response or resistance. For the exemplary study outlined here, we employed an “all-comers” approach, testing all the available NSCLC models at START Discovery, regardless of target expression level or molecular phenotype.

### 5.1 Methods

Mouse PDX trials were carried out at South Texas Accelerated Research Therapeutics (START, San Antonio, TX). START is accredited by AAALAC International (Association for the Assessment and Accreditation of Laboratory Animal Care International) and is compliant with the AstraZeneca Global Standard on Animal Care and Welfare. All models were developed at START. Patient-derived xenograft (START-PDX) models were established from viable human tumor tissue or fluid and have been serially passaged in animals a limited number of times to maintain tumor heterogeneity. Athymic Nude (Crl:NU(NCr)-Foxnlnu)/CB-17 Scid (CB17/Icr-Prkdcscid/IcrIcoCrl) mice were implanted unilaterally on the flank with tumor fragments harvested from host animals, each implanted from a specific passage lot. Pre-study tumor volumes were recorded beginning approximately one week prior to its estimated start date. When tumors reached the appropriate Tumor Volume Initiation (TVI) range (125-250 mm³), animals were randomized into treatment and control groups and intravenous (IV) dosing was initiated (Day 0); animals were followed individually throughout the study. Initial dosing began on Day 0; animals in all groups were dosed I.V. by weight (0.01 ml per gram; 10 ml/kg). Drug treated animals were dosed every 7 days for a total of 4 doses. Beginning on Day 0, tumor dimensions were measured by digital caliper and estimated tumor volumes were recorded for each treated and control animal; tumor volume was calculated using the formula: TV=width²×length×0.52. Tumor growth observations continued for one week after the final dose. Each animal was sacrificed upon reaching the Tumor Volume (TV) endpoint (tumor volume ≥1 cm³) or the study time endpoint of 28 days, whichever came first. The observation period was extended for some PDX models with slow growing tumors. Tumor growth inhibition (% TGI) was defined as Percent tumor growth versus Day 0 between treatment (TX) and control (C) groups, according to the formula: % TGI=1−(TX_{final}-TXᵢₙᵢₜᵢₐₗ)/(C_{final}−Cᵢₙᵢₜᵢₐₗ). Percent Tumor Regression was defined as the percentage tumor reduction of tumors in treated animals relative to the Day 0 tumor volume (day of initial dose), calculated at study endpoint according to the following formula: % Regression=(TX_{final avg}−TX_{initial avg})/(TX_{initial avg})×100.

### 5.2 Results and Conclusions

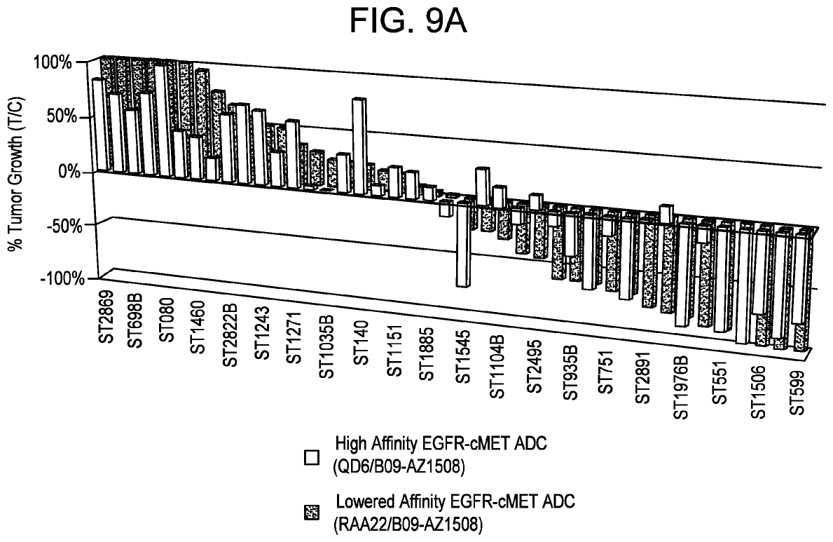

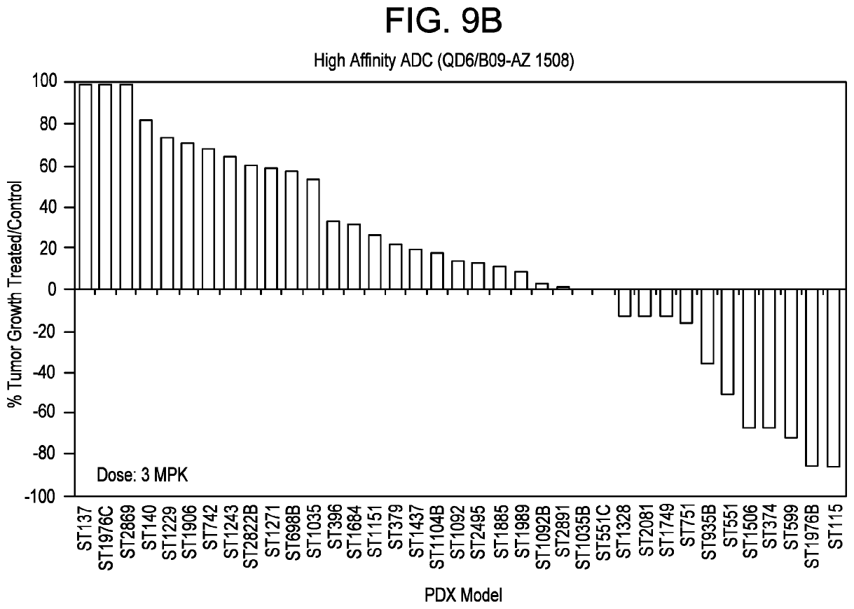

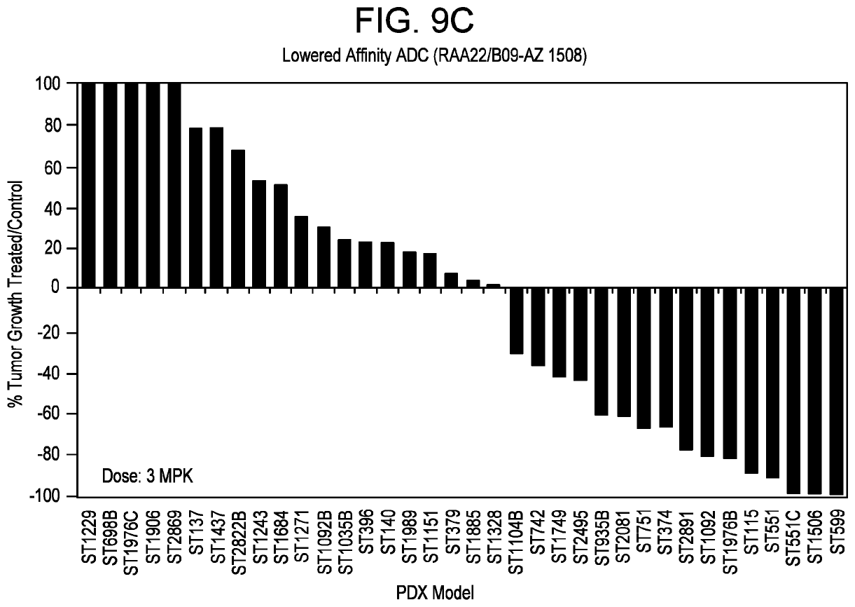

As shown in FIG. 9, both the high affinity EGFR-cMET ADC, QD6/B09-AZ1508, and the variant with lowered affinity for EGFR, RAA22/B09-AZ1508, induced tumor growth inhibition or regressions in numerous PDX models tested. Surprisingly, the lowered affinity ADC showed an overall trend of increased number and depth of responses observed, compared to the higher affinity ADC. This activity trend was slightly reversed for the PDX models that were least responsive to the lowered affinity ADC, which correlated somewhat to lower cMET expression. These observations raise the possibility that activity of the lowered affinity EGFR-cMET ADC is partially driven by cMET expression levels. The EGFR binding arm of both bispecific antibodies was derived from the same mouse EGFR cross reactive antibody (see Example 1). The intrinsic binding affinity of the QD6/B09 antibody toward mouse EGFR was approximately 6 nM, whereas the affinity of the RAA22/B09 bispecific antibody was approximately 575 nM. The unexpected improvement in the activity of the lowered EGFR affinity could be attributed to a reduced impact from the EGFR sink in normal tissues, such as the skin, resulting in higher overall circulating exposure of the ADC. Regardless, these data demonstrate that reducing the affinity toward EGFR of the EGFR-cMET bispecific antibody did not compromise the in vivo efficacy of the resulting ADC, but unexpectedly improved the activity compared to the higher affinity ADC.

### 5.3 PDX Study at Different Doses

A further experiment was carried out to test different doses of the ADCs in PDX models. This experiment was carried out as described in Example 5.1, except that individual mice reaching a tumor volume of ≥2 cm³ were removed from the study and the final measurement included in the group mean until the mean reached volume endpoint or the study reached the time endpoint of 63 days. For the exemplified study, the high affinity EGFR-cMET ADC, QD6/B09-AZ1508, was tested at dose levels of 1 and 2 mg/kg and the variant with lowered affinity for EGFR, RAA22/B09-AZ1508, was tested at 1, 2, and 3 mg/kg.

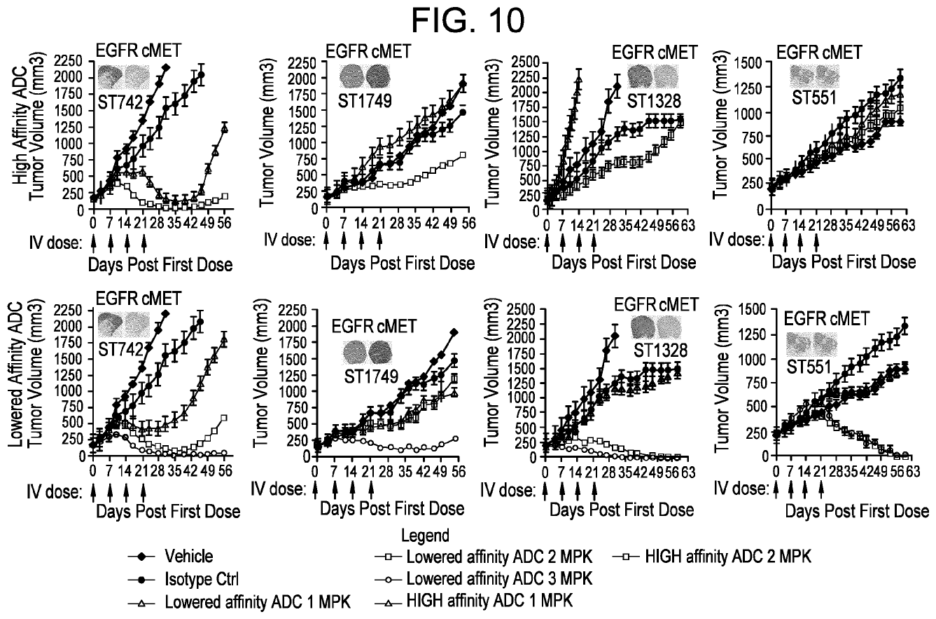

As demonstrated in FIG. 10, both the high affinity EGFR-cMET ADC, QD6/B09-AZ1508, and the variant with lowered affinity for EGFR, RAA22/B09-AZ1508, induced tumor growth inhibitory activity in PDX models at the tested doses. In accordance with the results described in Example 5.2 and shown in FIG. 9, the lowered affinity ADC was generally more efficacious than the high affinity ADC. In all four models tested, the lowered affinity ADC induced regressions at 2 or 3 mg/kg dose levels, demonstrating that the lowered affinity ADC is efficacious at modest doses. These data provide further evidence that reducing the affinity of the EGFR-cMET bispecific antibody toward EGFR did not reduce the in vivo efficacy of the resulting ADC, but rather improved the in vivo efficacy compared to the higher affinity ADC.

### Example 6—ADC Efficacy in Orthotopic Pancreatic PDX Model

Subcutaneous in vivo tumor models are the mainstay for examining the efficacy of anti-cancer agents. However, this tumor implantation site is accompanied by a number of limitations that need to be considered when interpreting in vivo results. These deficiencies include, tumor vascularization and the lack of tissue-specific stroma in the growth and response of the tumor. To address these challenges, we compared the in vivo efficacy of both the High and Low affinity EGFR-cMET bispecific Antibody-Drug Conjugates in both subcutaneous and orthotopic models of a pancreatic PDX model MEDI-PANC-08. To track the growth of this tumor orthotopically, we developed a Luciferase expressing PDX variant (MEDI-PANC-08^{LUC}) whose growth could be tracked using Imaging.

### 6.1 Methods

All experiments were conducted in an AAALAC (Association for Assessment and Accreditation of Laboratory Animal Care) accredited facility, in accordance with MedImmune's IACUC (Institutional Animal Care and Use Committee) guidelines for humane treatment and care of laboratory animals. Animals were monitored daily for morbidity and mortality.

### Subcutaneous PDX Model

The MEDI-PANC-08 pancreatic PDX model used in this study came from the Internal MedImmune PDX library. The PDX tumor was initially propagated in seed NSG (NOD·Cg-Prkdc^{scid}Il2rg^{tmiWj1}/SzJ) mice, to generate sufficient tumor material to seed the efficacy study. Once tumors reached 800-1200 mm³ the mice were humanely euthanized by CO₂ asphyxiation. Tumors were isolated under sterile conditions, cut into ˜2 mm³ pieces and implanted subcutaneously into the right flank of individual NSG mice using an 11-gauge trocar needle. Upon reaching ˜150-250 mm3 in size, mice were randomized (based on tumor volume) into treatment groups and treated with the ADCs (Q1W×4). Two EGFR-cMET bispecific ADCs were examined at 1, 2 and 3 mg/kg the QD6/B09 (high affinity) and RAA2/B09 (low affinity). An Isotype control ADC (R347-AZ1508) was also tested at 3 mg/kg. All Antibody-Drug Conjugates were diluted in buffer (25 mM Histidine, 7% Sucrose, 0.02% PS80, pH 6.0), immediately prior to use and administered i.v. via the tail vein. Tumor and body weight measurements were collected twice weekly and tumor volume calculated using the equation (L×W2)/2, where L and W refer to the length and width dimensions, respectively.

### Orthotopic PDX Model

The luciferase expressing PDX model (MEDI-PANC-08^{LUC}) was grown subcutaneously in NSG seed mice and at a volume of 800-1200 mm³ the tumors were harvested and cut into fragments of approximately 2 mm³. The tumor fragments were subsequently sutured to the pancreas of NSG mice (Day Zero). Luciferase signal was determined weekly using the IVIS Spectrum In vivo Imaging system. Briefly, 10 minutes prior to imaging, 200 ul of luciferin dissolved in DPBS (15 mg/ml) was injected intra-peritoneally (i.p.). The mice were anesthetized under 3% isoflurane, laid on their right side and luminescence measured. Fourteen days after tumor implant, when luminescent signal was clearly detectable, the mice were randomized into their respective groups based on the luminescence. The mice were treated with Isotype control (R347-AZ1508, 3 mg/kg—Q1W×4), Gemcitabine (75 mg/kg, Q2D×5) and RAA2/B09 ADC (2 and 3 mg/kg—Q1W×4). Luminescence was measured weekly. Study endpoints included body weight loss, deterioration of body condition and lethargy. Data were analyzed using the Living Image software (Perkin Elmer) and plotted as Average Radiance [p/s/cm2/sr] against time.

### 6.2 Results and Discussion

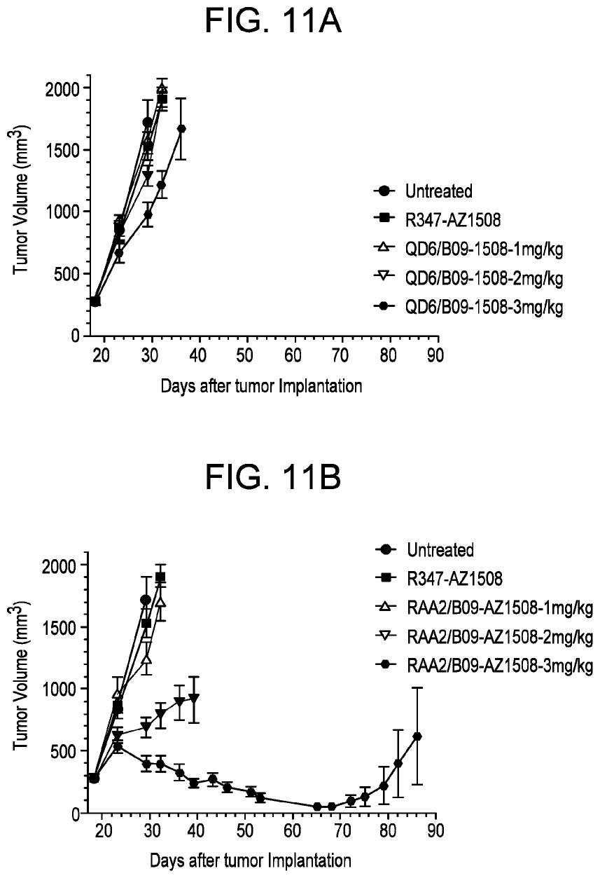

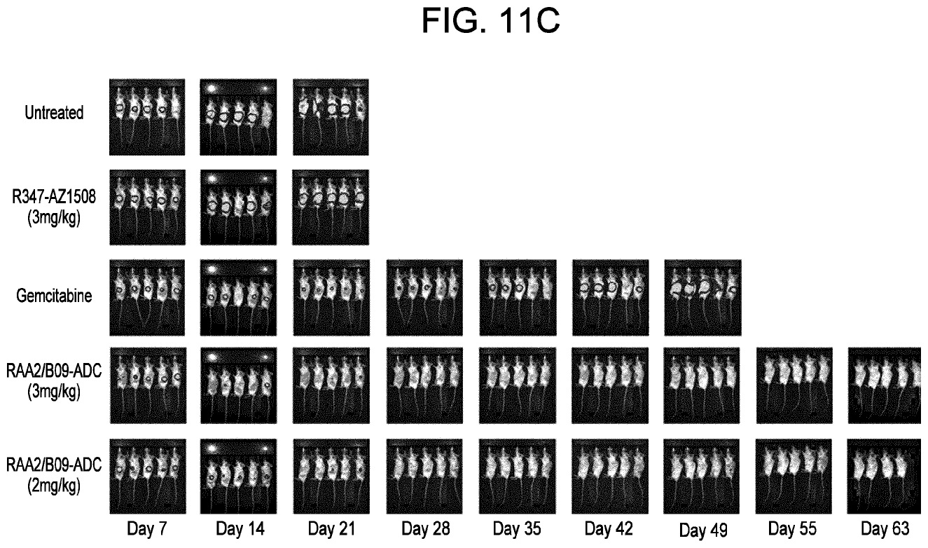

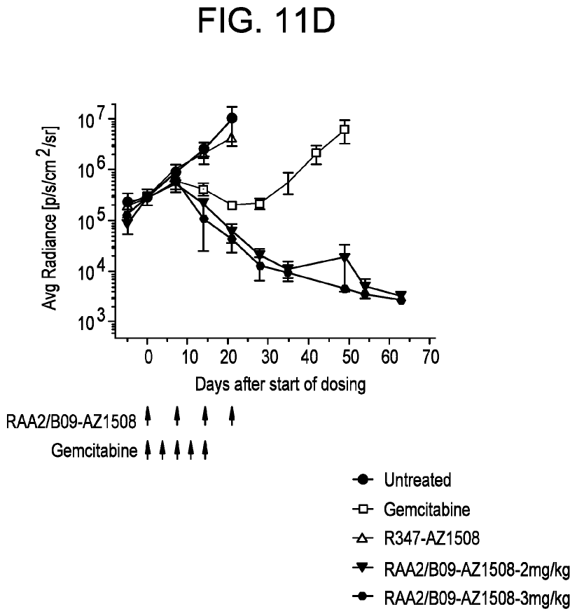

To assist in the selection of the appropriate EGFR affinity combination for the EGFR-cMET bispecific ADC, high and low affinity EGFR-cMET bispecific ADCs were compared in an in vivo efficacy study using the MEDI-PANC-08 pancreatic PDX model. As shown in Panels A and B of FIG. 11, a disparate difference was observed between the 2 molecules. The high affinity QD6/B09 ADC did not show efficacy at any of the 3 dose levels tested. Conversely, the low Affinity RAA2/B09 ADC produced complete tumor regression by day 65 followed by tumor re-growth at the 3 mg/kg dose level and tumor growth inhibition at 2 mg/kg.

Whilst subcutaneous tumor models have become the work-horse for in vivo efficacy studies, a major deficiency is that tumors are not grown at the site of origin and hence any drug response might not truly reflect of how patients will respond. To address this concern, an orthotopic model of pancreatic cancer was developed using the MEDI-PANC-08 tumor that had been transgenically modified to stably express luciferase. Following surgical implantation on the pancreas, tumors were allowed to establish and subsequently randomized based on luminescent signal. The mice were then treated with either the Low affinity RAA2/B09 EGFR-cMET ADC, isotype control or gemcitabine (a chemotherapy drug). Following treatment, the luminescence was measured weekly. As shown in Panel C of FIG. 11 the luminescence in the untreated and isotype control groups increases over time with animals removed from study due to poor body condition and large, palpable abdominal tumors. Gemcitabine showed an initial reduction in the luminescent signal reaching a nadir around day 21, after which the signal increased over time with the group removal from study at day 49. In the orthotopic model, both the 2 and 3 mg/kg dose levels of RAA2/B09 ADC caused a reduction in luminescent signal reaching close to background levels by day 60. In comparison to the subcutaneous study, the 2 mg/kg dose level demonstrated better activity producing tumor regressions. Necropsy of animals at the end of the study that showed tumors with luminescent signals close to background no longer showed visible tumor, hence supporting the correlation between luminescent signal and tumor volume.

In conclusion, the low Affinity EGFR-cMET RAA2/B09 ADC demonstrated improved efficacy compared to the high affinity QD6/B09 ADC in a subcutaneous PDX Pancreatic PDX model, with tumor regressions seen at 3 mg/kg. This efficacy was also observed in an orthotopic model using the same PDX tumor (MEDI-PANC-08) engineered to stably express luciferase. Using luminescence as a surrogate for tumor volume, RAA2/B09 ADC showed improved efficacy over the subcutaneous model producing tumor regressions at both 2 and 3 mg/kg.

### Example 7—Safety and Pharmacokinetics

Pharmacokinetic (PK) analyses were carried out to compare the plasma PK parameters of the low and high affinity EGFR-cMET ADCs, including peak and total exposure, clearance, and half-life in mice and cynomolgus monkeys. A key aim was to determine whether reducing the affinity for EGFR would impact the circulating exposure of the EGFR-cMET bispecific ADC. PK samples were collected in mice and cynomolgus monkeys for both QD6/B09-57-AZ1508 and RAA/B09-57-AZ1508 across various dose levels. Non-compartmental analysis was performed to estimate PK parameters for QD6/B09-57-AZ1508 and RAA22/B09-57-AZ1508 based on total ADC concentrations across species and dose levels.

Overall, RAA22/B09-57-AZ1508 shows higher exposure and prolonged t1/2 compared to QD6/B09-57-AZ1508 in both mice and cynomolgus monkeys, suggesting improved PK in the lower affinity RAA22/B09-57-AZ1508.

### 7.1 Bioanalysis of Pre-Clinical PK Assay

The target compounds (QD6/B09-57-AZ1508 and RAA22/B09-57-AZ1508) concentration and the total antibody concentration was measured with one immuno capture LC-MS/MS assay. Briefly, a polyclonal anti-human antibody was conjugated to magnetic beads. Then 25 μL of plasma sample was diluted in PBS and incubated together with the magnetic beads. After capturing, the magnetic beads were washed multiple times before digested with trypsin under the presence of internal standards. The digestion was quenched with the addition of acid. The liquid content was then transferred to the injection plate.

The signature tryptic peptide on the human antibody Fc region and the cleaved warhead was separated using reversed phase chromatography (RPLC) followed with detection using multiple reaction monitoring (MRM). A signature peptide on the Fc region was used to calculate total Ab, while the digestion released warhead was used to calculate the ADCs. The internal standard used in this experiment are isotopically labeled peptide or protein (SiluMAb, Sigma-Aldrich) or isotopically labeled warhead. The peak area ratio of the analyte against the internal standards was used to calculate against the standard curve.

The standard curves and QCs are prepared by spiking the target compounds at different levels into the same matrix as the samples. The quantification range covers 100 ng/mL-15,000 ng/mL, with the dilution QC covering up to 525,000 ng/mL. The standard curve was fitted with the simplest possible model. The accuracy and precision of the assay is within 20% for all levels except the lower limit of quantification (LLOQ), which is at 25%.

### 7.2 QD61B09-57-AZI508 PK in Mice

Mice studies included in the NCA analysis are summarized in Table 4.

TABLE 4
List of mice studies in RAA22/B09-57-AZ1508 and QD6/B09-57-AZ1508

| Study | Study Tile | Study design |
|---|---|---|
| 03-EGFR cMET- | Pharmacokinetics Study of | Single IV dose at 1, 3, 5, and 10 mg/kg; |
| ADC-PK-15-003-A | cMET-EGFR-AZ1508 in Nude | Plasma collections at 0.5, 4, 24, 48, 72, |
| | Mice | 144, & 240 hr; LC-MS/MS analysis of |
| | (QD6/B09-57-AZ1508) | ADC and total Ab |
| RAA22/B09- | Pharmacokinetics Study of | Single IV dose at 0.5, 1, 3, 5, and 10 |
| AZ1508 mouse PK | cMET-EGFR DuetMAb | mg/kg; Plasma collections at 0.5 hr, 4 hr, |
| study - October | RAA22/B09-AZ1508 in Nude | 1 d, 2 d, 3 d, 6 d, 10 d, & 14 d; LC-MS/MS |
| 2016 | Mice | analysis of ADC and total Ab |

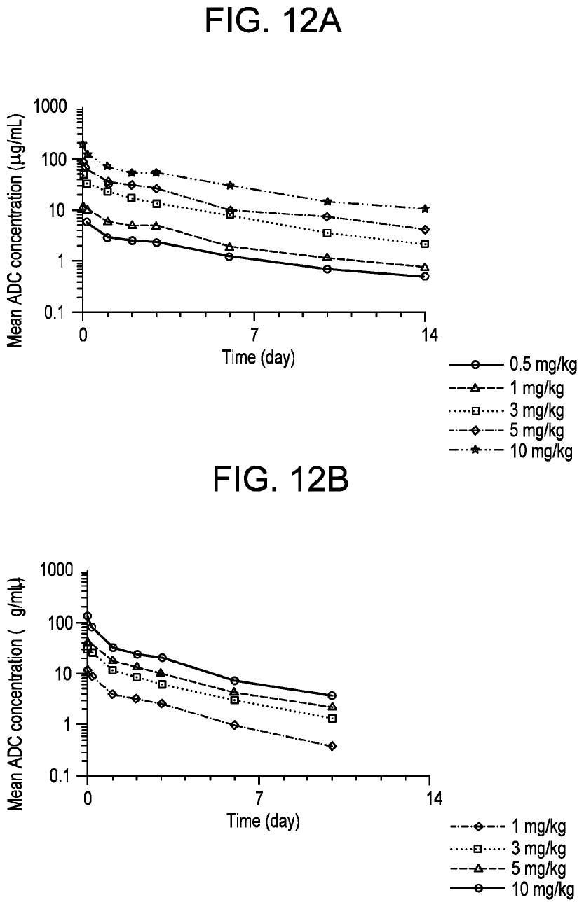

Mean PK concentration-time profiles in mice for RAA22/B09-57-AZ1508 and QD6/B09-57-AZ1508 are presented in FIG. 12.

Both RAA22/B09-57-AZ1508 and QD6/B09-57-AZ1508 exhibited linear PK in mice at the dose levels tested, with dose-proportional exposure (Cₘₐₓ and AUC), comparable CL and t1/2 observed at 0.5 mg/kg to 10 mg/kg for RAA22/B09-57-AZ1508 and at 1 mg/kg to 10 mg/kg for QD6/B09-57-AZ1508, respectively.

PK comparison between RAA22/B09-57-AZ1508 and QD6/B09-57-AZ1508 is assessed at 1, 3, 5, 10 mg/kg dose levels that were tested for both compounds and the mean PK parameters based on NCA is summarized in Table 5. The results demonstrated slower CL, higher exposure and prolonged t1/2 in RAA22/B09-57-AZ1508, with mean AUC of RAA22/B09-57-AZ1508 showed 2- to 2.91-fold increase compared to QD6/B09-57-AZ1508; the mean t1/2 ranged from 4.24 to 6.38 days for RAA22/1509-57-AZ1508 and from 2.57 to 3.33 days for QD6/709-57-AZ1508, represented 1.27- to 2.32-fold increase in t1/2 for RAA22/1B09-57-AZ1508.

TABLE 5
Mean NCA PK parameters by Dose levels between RAA22/B09-57-AZ1508 and QD6/B09-57-AZ1508 in Mice

| | Dose (mg/kg) | | | | | | | |
|---|---|---|---|---|---|---|---|---|
| | 1 mg/kg | | 3 mg/kg | | 5 mg/kg | | 10 mg/kg | |
| | molecule | | | | | | | |
| | RAA22/B09- | QD6/B09- | RAA22/B09- | QD6/B09- | RAA22/B09- | QD6/B09- | RAA22/B09- | QD6/B09- |
| | 57-AZ1508 | 57-AZ1508 | 57-AZ1508 | 57-AZ1508 | 57-AZ1508 | 57-AZ1508 | 57-AZ1508 | 57-AZ1508 |
| Cₘₐₓ | 12.8 | 11.3 | 51.1 | 31.0 | 90.7 | 44.8 | 198 | 134 |
| (mg/mL) | | | | | | | | |
| AUC | 41.0 | 20.5 | 136 | 57.5 | 230 | 87.9 | 494 | 170 |
| (mg*day/mL) | | | | | | | | |
| t½ (day) | 5.97 | 2.57 | 4.24 | 3.33 | 6.38 | 3.28 | 5.31 | 2.95 |
| CL | 20.8 | 45.7 | 20.0 | 46.9 | 18.5 | 50.8 | 17.3 | 53.8 |
| (mL/kg/day) | | | | | | | | |

### 7.3 QD61B09-57-AZI508 PK in Cynomolgus Monkeys

Cynomolgus monkey studies included in the NCA analysis is summarized in Table 6.

TABLE 6
List of cynomolgus monkey studies in RAA22/B09-
57-AZ1508 and QD6/B09-57-AZ1508

| Study | Study Tile | Study design |
|---|---|---|
| 20102256 | RAA22/B09-Maia-AZ1508: An | IV bolus injection Q3 Wk × 2 |
| | Intravenous Repeat Dose Toxicity Study to | |
| | Evaluate the Safety and Pharmacokinetics | |
| | in Cynomolgus Monkeys | |
| 20067678 | An 8-Week Dose Range Finding Study of | IV bolus injection Q3 Wk × 3 |
| | EGFR cMET by Intravenous Bolus | |
| | Injection in Cynomolgus Monkeys | |
| 20067312 | A 8-Week Single Ascending Dose of | Single ascending IV dosing |
| | EGFRcMet-ADC (EGFRcMet-1508) by | |
| | Intravenous Route in Cynomolgus | |
| | Monkeys | |

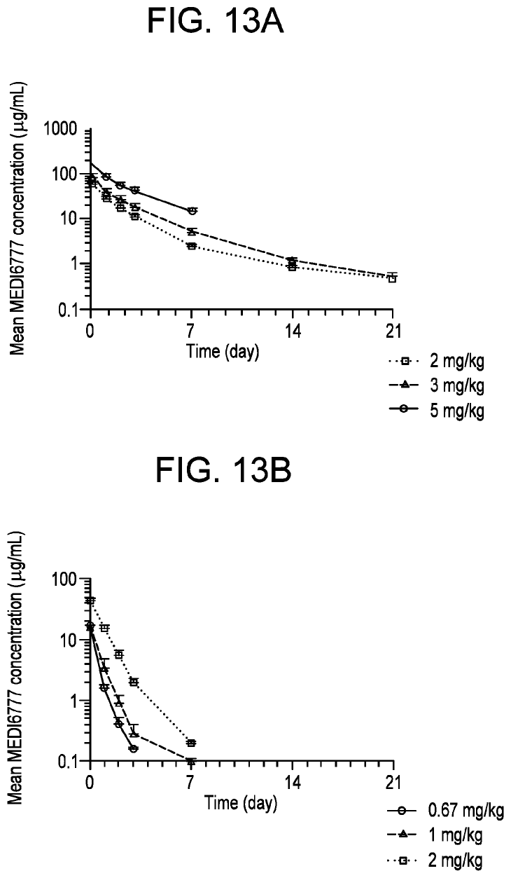

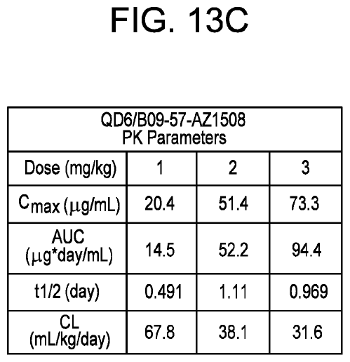

Mean PK concentration-time profiles in Cynomolgus monkeys for RAA22/B09-57-AZ1508 and QD6/B09-57-AZ1508 is presented in FIG. 13.

RAA22/B09-57-AZ1508 exhibited linear PK in cynomolgus monkeys at 2 mg/kg to 5 mg/kg, with dose-proportional exposure (Cₘₐₓ and AUC), comparable CL and t1/2 observed.

QD6/B09-57-AZ1508 exhibited non-linear PK in cynomolgus monkeys at 0.67 mg/kg to 3 mg/kg, with more than dose-proportional exposure (Cₘₐₓ and AUC) shown, and faster CL and shorter t1/2 observed at lower dose levels.

PK comparison between RAA22/B09-57-AZ1508 and QD6/B09-57-AZ1508 in cynomolgus monkeys is assessed at 2 and 3 mg/kg dose levels that were tested for both compounds and the mean PK parameters based on NCA is summarized in Table 7. The results demonstrated slower CL, higher exposure and prolonged t1/2 in RAA22/B09-57-AZ1508, with mean AUC of RAA22/B09-57-AZ1508 showed 1.90- to 2.43-fold increase compared to QD6/B09-57-AZ1508; The mean t1/2 were 4.30 to 5.90 days for RAA22/B09-57-AZ1508 and 0.969 to 1.07 days for QD6/B09-57-AZ1508, represented 4.44- to 5.51-fold increase in t1/2 for RAA22/B09-57-AZ1508.

TABLE 7
Mean NCA PK parameters by Dose levels between RAA22/B09-
57-AZ1508 and QD6/B09-57-AZ1508 in Monkeys

| | 2 mg/kg | | 3 mg/kg | |
|---|---|---|---|---|
| Dose (mg/kg) | RAA22/B09- | QD6/B09- | RAA22/B09- | QD6/B09- |
| molecule | 57-AZ1508 | 57-AZ1508* | 57-AZ1508 | 57-AZ1508 |
| Cₘₐₓ | 59.1 | 47.5 | 86.6 | 73.3 |
| (mg/mL) | | | | |
| AUC | 116 | 47.6 | 179 | 94.4 |
| (mg*day/mL) | | | | |
| t½ (day) | 5.90 | 1.07 | 4.30 | 0.969 |
| CL | 16.7 | 42.2 | 16.5 | 31.6 |
| (mL/kg/day) | | | | |

Taken together, these data demonstrate that the low affinity RAA22/B09-57-AZ1508 shows higher exposure and increased circulating half life compared to the high affinity QD6/B09-57-AZ1508 in both mice and cynomolgus monkeys. These data are consistent with the hypothesis that reducing the affinity for EGFR reduces the binding to EGFR present in normal tissue, thereby lessening the impact of the normal tissue sink and improving the plasma PK parameters.

### Example 8—ADC with Topoisomerase I Inhibitor as Payload

An experiment was designed to test the efficacy and safety of the RAA22/B09-57 bispecific molecule conjugated to a different payload—a topoisomerase I inhibitor rather than the tubulysin used in the previous examples.

The DuetMab RAA22/B09 (with the “Maia” cysteine insertion after serine 239) bispecific antibody produced according to Example 1 was conjugated to the topoisomerase inhibitor SG3932 via “classical” conjugation to native cysteines in the bispecific antibody.

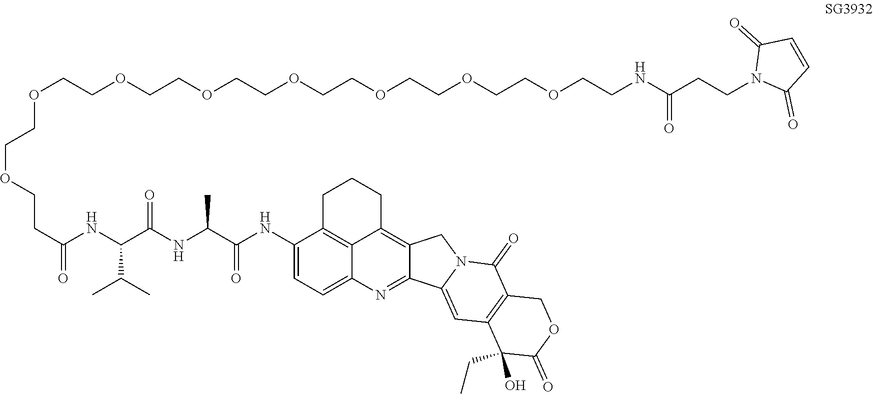 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

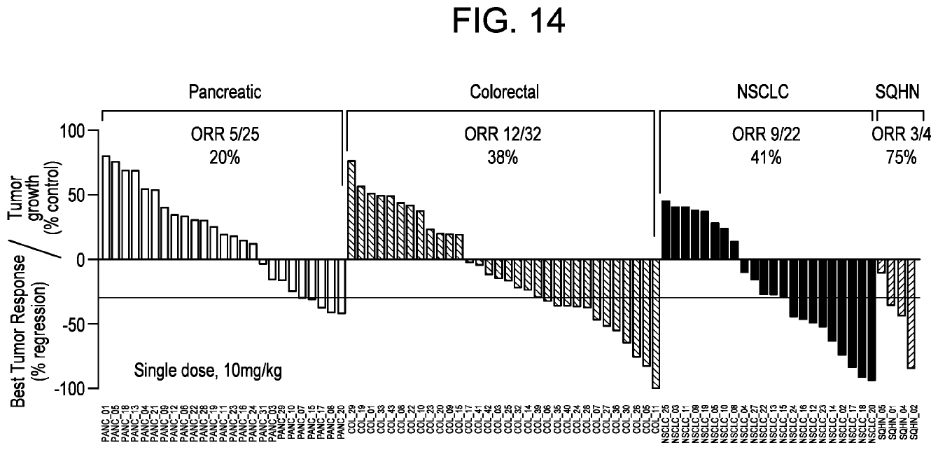

The efficacy of the EGFR/cMET topoisomerase I inhibitor ADC was investigated using a PDX trial. The PDX trial was carried out essentially as described above for Example 5 using a variety of different PDX models obtained from pancreatic, colon, NSCLC and squamous head and neck carcinoma (SQHN) tumors. Animals were injected with a single dose of the EGFR-cMET Maia Topo ADC at 10 mg/kg. The results of the PDX trial using the EGFR-cMET Maia Topo ADC are reported in FIG. 14.

As shown in FIG. 14, the EGFR-cMET Maia Topo ADC induced tumour growth inhibition or regression in numerous PDX models tested. Thus, these results demonstrate that RAA22/B09-57ADC containing the topoisomerase I inhibitor was efficacious in the PDX models representing multiple tumor types.

### Example 9—Mutations to Improve PK of the ADC

The ADC with the topoisomerase I inhibitor produced and tested in Example 8 used the RAA22/B09 bispecific antibody containing the “Maia” cysteine insertion after serine 239. However, given that SG3932 conjugates to native cysteines, the Maia cysteine insertion is not necessary. We therefore sought to modify the RAA22/B09 Maia Topo ADC produced in Example 8 to remove this cysteine insertion.

It was also recognised that it may be possible to mitigate immune toxicities and improve pharmacokinetics if the effector functions of the Fc backbone were reduced or removed. We therefore also introduced the “triple Mutant™” of L234F/L235E/P331S (EU numbering) that has previously been shown to reduce Fc effector functions in antibody molecules (Organesyan, 2008; Hay, 2016).

The newly generated “EGFR-cMET™” molecule, comprising variable regions from RAA22 and B09, with the 239i mutation removed and the TM introduced has the amino acid sequences set forth in the following table:

| | EGFR-cMET TM |
|---|---|
| EGFR heavy chain | 59 |
| c-Met heavy chain | 60 |
| EGFR light chain | 61 |
| c-Met light chain | 62 |

For conjugation to SG3932, a 50 mM solution of Tris(2-carboxyethyl)phosphine (TCEP) in phosphate-buffered saline pH 7.4 (PBS) was added (12.5 molar equivalent/antibody) to a solution of EGFR-cMET™ bispecific antibody in reduction buffer containing PBS and 1 mM ethylenediaminetetraacetic acid (EDTA) and a final antibody concentration of ˜3 mg/mL. The reduction mixture was allowed to incubate at 37° C. for 2 h in an orbital shaker with gentle (60 rpm) shaking. Reaction mixture was allowed to cool down to room temperature for 45 min. SG3932 was then added as a DMSO solution (12.5 molar equivalent/antibody) for a 10% (v v) final DMSO concentration. The solution was incubated for 2 hours at room temperature and then quenched by the addition of N-acetyl cysteine (5 micromoles/SG3932) and incubated at room temperature for 15 min. The reaction mixture filtered using 0.2 uM sterile filter and then stored at 2-8° C. overnight. Excess free drug was removed via Tangential Flow Filtration unit (TFF) using mPES, MidiKros® 30 kDa fiber filter with 375 cm² surface area, into buffer containing 30 mm Histidine, 30 mM Arginine, pH 6.8. Extent of free drug removal was monitored by UHPLC-RP using neat conjugate. After complete removal of free drug, ADC was buffer exchanged. ADC was filtered using 0.22 μm filter under sterile atmosphere and then polysorbate-80 was added to a final concentration of 0.02% (w/v).

UHPLC analysis on a Shimadzu Prominence system using a Thermo Scientific MAbPac 50 mm×2.1 mm column eluting with a gradient of water and acetonitrile on a reduced sample of ADC at 214 nm and 330 nm (SG3932 specific) revealed a drug-per-antibody ratio (DAR) of 6.0 molecules of SG3932 per antibody.

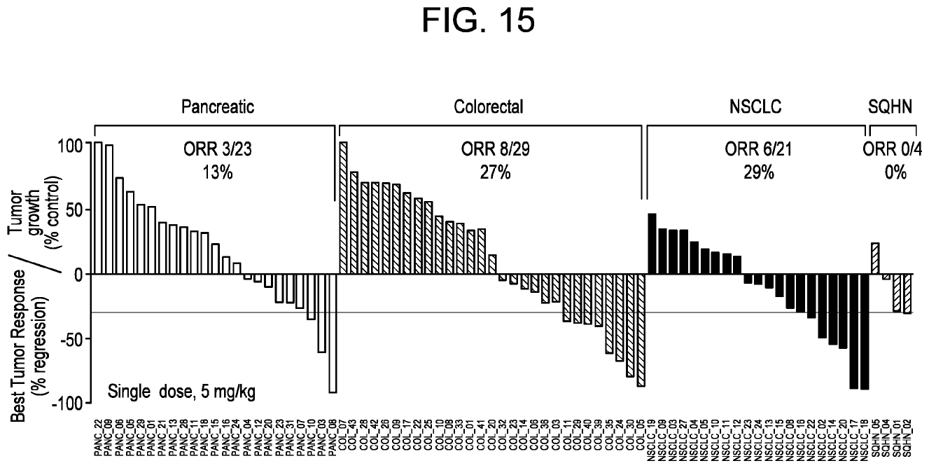

The efficacy of the EGFR-cMET™ ADC was investigated using a PDX trial. The PDX trial was carried out essentially as described above for Example 5 using a range of different PDX models obtained from pancreatic, colon, NSCLC and SQHN tumours. Animals were injected with a single dose of the EGFR-cMET™ ADC at 5 mg/kg. The results of the PDX trial using the EGFR-cMET™ ADC are reported in FIG. 15. Results from this experiment demonstrate that the EGFR-cMET™ ADC was able to induce tumour growth inhibition or regression in numerous PDX models tested

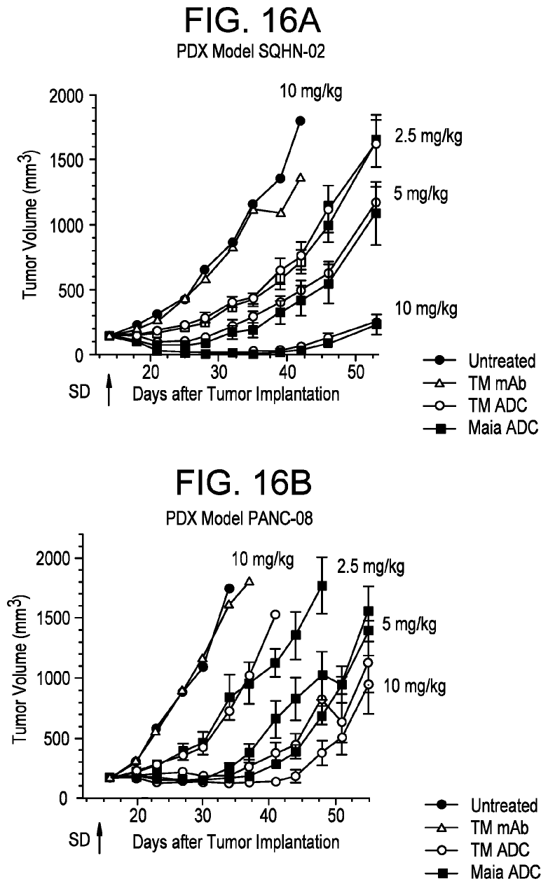

The efficacy of the EGFR-cMET™ ADC (“TM ADC”) was compared to the EGFR-cMET Maia Topo ADC (“Maia ADC”) produced in Example 8 in PDX models SQHN-02 and PANC-08. The animals were doses with 2.5 mg/kg, 5 mg/kg or 10 mg/kg of each ADC and tumour growth monitored. Also included in this experiment was an untreated control (“untreated) and animal dosed with unconjugated EGFR-cMET™ (TM mAb). The results are shown in FIG. 16.

The results demonstrate that both EGFR-cMET™ ADC and EGFR-cMET Maia Topo ADC are similarly efficacious (“equipotent”) at reducing tumour growth/inducing tumour regression at the range of doses tested. This suggests that the removal of the S239i mutation and abrogation of the Fc effector functions using the triple mutant does not negatively affect efficacy in these tumour models.

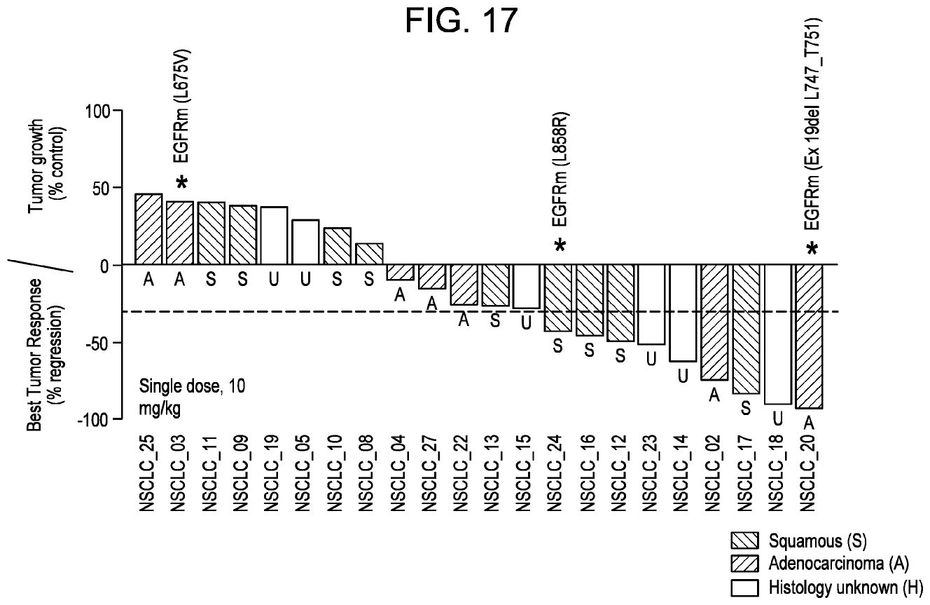

The EGFR-cMET™ ADC was also shown to be effective at reducing tumour growth in NSCLC tumours that express either wild type or mutant EGFR. Results demonstrate that the EGFR-cMET™ ADC is active in both wild type and mutant EGFR PDX models are shown in FIG. 17. This is advantageous, as it indicates that the ADCs will be able to provide a benefit in multiple therapeutic settings and across a range of different EGFR genotypes.

Further, pharmacokinetic (PK) studies were carried out in NOD-SCID mice to compare the EGFR-cMET™ ADC (“TM ADC”) and the EGFR-cMET Maia Topo ADC (“Maia ADC”) produced in Example 8. The Experiment was carried out essentially as described in Example 7.

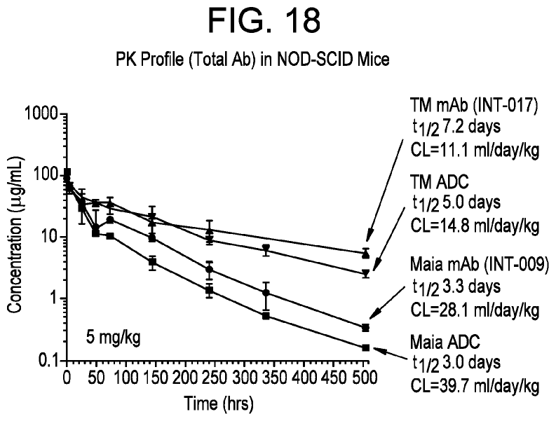

Representative results of these PK studies are provided in FIG. 18. As reported in this figure, the EGFR-cMET™ ADC exhibited a greater half-life (t1/2=5.0 days) and reduced drug clearance (CL=14.8 ml/day/kg) when compared to the EGFR-cMET Maia Topo ADC (t1/2=3.0 days; CL=39.7 ml/day/kg). Thus, the results indicate that the EGFR-cMET™ ADC described here shows improved PK compared to the EGFR-cMET Maia Topo ADC produced in Example 7. A similar improvement in PK was also observed when comparing the unconjugated EGFR-cMET™ antibody (“TM mAb”) to the unconjugated EGFR-cMET Maia (“Maia ADC”).

### Example 10—ADC Efficacy in Combination with Osimertinib

This example compares the ADC efficacy in combination with the third generation tyrosine kinase inhibitor TKI osimertinib (‘Osi’) in various EGFR mutant tumour models using a PDX trial.

### 5.1 Methods

Preclinical efficacy studies in immunocompromised mice or bearing patient-derived xenografts (PDX) were carried out at Champions Oncology (athymic nude-Foxn1nu mice) and Genendesign (Balb/C nude mice). All studies were compliant with the AstraZeneca Global Standard on Animal Care and Welfare. Models were established from viable human tumor tissue or fluid and have been serially passaged in animals a limited number of times to maintain tumor heterogeneity. Mice were implanted unilaterally on the flank with tumor fragments harvested from host animals, each implanted from a specific passage lot. Pre-study tumor volumes were recorded beginning approximately one week prior to its estimated start date. When tumors reached the appropriate Tumor Volume for Initiation (TVI) range (150-300 mm³), animals were randomized into treatment and control groups.

Treatments—Animals receiving either EGFR-cMET Top Ii antibody drug conjugate (ADC) described in Example 9 or the control EGFR-cMET mAb were administered a single intravenous (IV) dose at the indicated dose level on Day 0; animals were dosed IV by weight (at a dose volume of 5 ml/kg). Starting on Day 0, animals in the osimertinib treatment group received the drug formulated in an oral dosing solution at 2.5 mg/ml in vehicle (0.5% w/v HPMC (hydroxyl propyl methyl cellulose) in deionised water); animals were dosed orally by weight at a dosing volume of 10 mL/kg to give a final dose level of 25 mg/kg. Osimertinib treated animals were dosed daily for the first 21 days of the study. Animals in the combination treatment groups received both the EGFR-cMET ADC and osimertinib, with each treatment administered according to the monotherapy dosing schedule described above. All animals were followed individually throughout the study.

Tumor Growth Inhibition—Beginning on Day 0, tumor dimensions were measured twice weekly by digital caliper and data including individual and mean estimated tumor volumes (Mean TV±SEM) were recorded for each group; tumor volume was calculated using the formula (1): TV=width2× length×0.52. At study completion, percent tumor growth inhibition (% TGI) values were calculated and reported for each treatment group (T) versus control (C) using initial (i) and final (f) tumor measurements by the formula (2): % TGI=1−(Tf−Ti)/(Cf−Ci). Individual mice reporting a tumor volume less than or equal to 30% of the Day 0 measurement for two consecutive measurements were considered partial responders (PR). Individual mice lacking palpable tumors (0.00 mm³ for two consecutive measurements) were classified as complete responders (CR); a CR that persisted until study completion were considered tumor-free survivors (TFS). Tumor doubling time (DT) were determined for the vehicle treated groups using the formula DT=(Df−Di)*log 2/(log TVf−log TVi) where D=Day and TV=Tumor Volume. Tumor growth observations in the untreated control group were carried out until the mean tumor volume of the group (uncensored) reached the humane endpoint of 1500 mm³, or until Day 60, whichever came first. Tumor growth observations in the treatment groups were carried out until Day 60; if tumors in individual mice in the treatment groups reached the humane endpoint of 1500 mm³, the animals were euthanized and observations in the other treatment animals continued. Some animals exhibiting a sustained response were observed beyond 60 days.

### Results

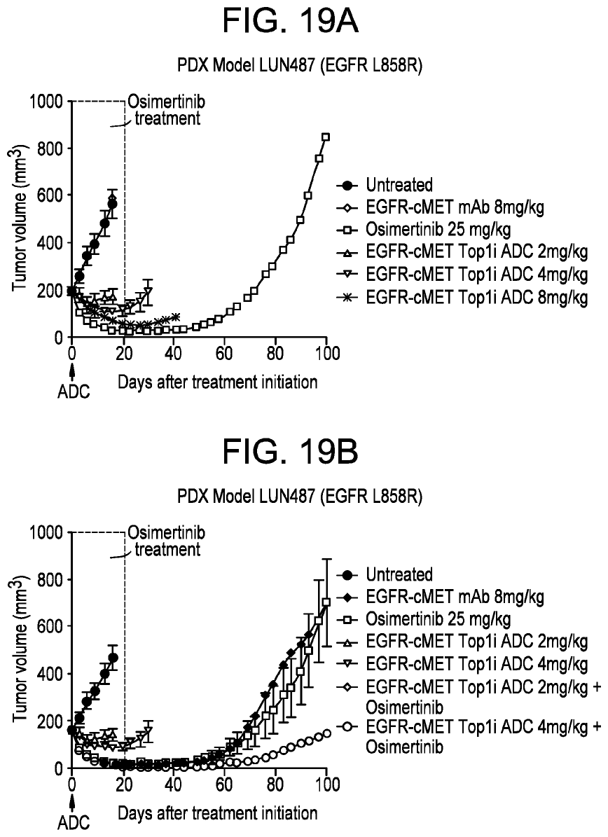

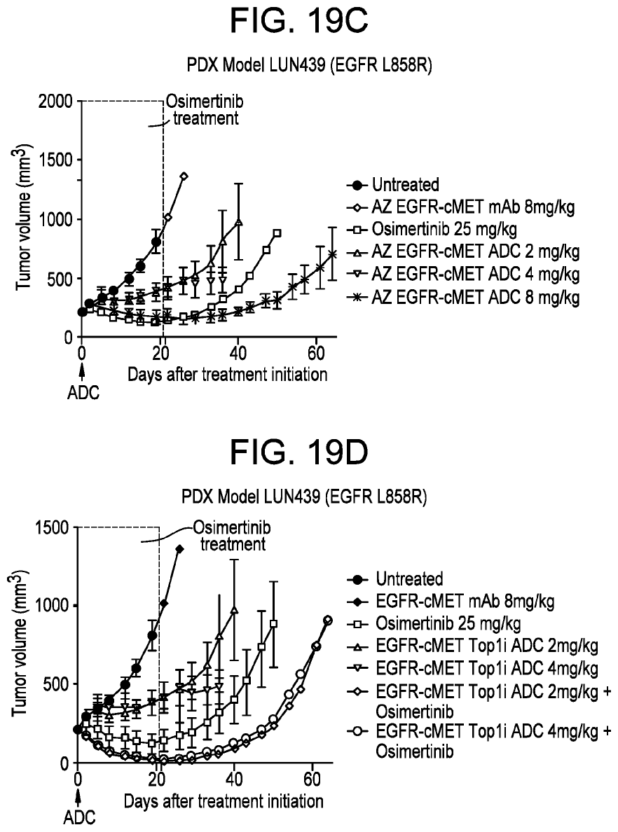

Results are provided in FIGS. 19-21.

FIGS. 19A and C shows the results for EGFR mutant models ‘LUN487’ and ‘LUN439’ containing the EGFR L858R mutation representing 1^{st} line EGFRm non-small cell lung cancer (NSCLC). The EGFR-cMET™ ADC was dosed at 2, 4 and 8 MPK (mg/kg). A group was also dosed with mAb-only control (EGFR-cMET mAb) at 8 mPK or osimertinib only at 25 MPK. The results show that EGFR-cMET™ ADC monotherapy showed a dose-dependent response. PDX models responded to osimertinib. No treatment response was observed for the mAb-only control.

FIGS. 19B and D shows the results for EGFR mutant models ‘LUN487’ and ‘LUN439’ where EGFR-cMET™ ADC was dosed at 2 or 4 MPK alone or in combination with Osimertinib (25 MPK). The combination of EGFR-cMET™ ADC and osimertinib demonstrated improved tumor growth inhibition compared to either agent (EGFR-cMET™ ADC or osimertinib) administered individually.

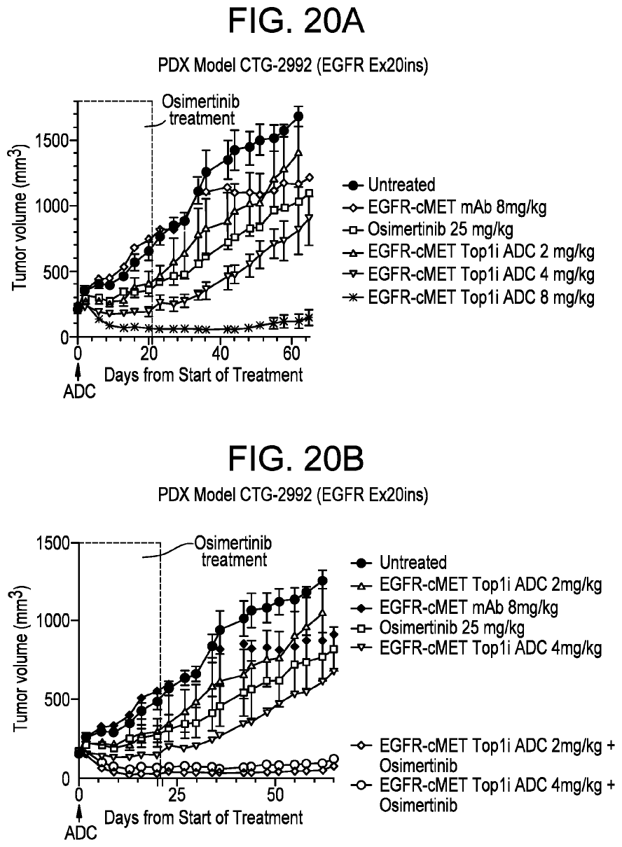

FIG. 20A shows the results for EGFR mutant model ‘CTG-2992’ containing an Exon20 insertion representing primary resistance to osimertinib. The EGFR-cMET™ ADC was dosed at 2, 4 and 8 MPK (mg/kg). A group was also dosed with mAb-only control (EGFR-cMET mAb) at 8 mPK or Osimertinib only at 25 MPK. The results show that EGFR-cMET™ ADC monotherapy showed a dose-dependent response. PDX models responded to Osimertinib.

FIG. 20B shows the results for EGFR mutant model ‘CTG-2992’ where the EGFR-cMET™ ADC and osimertinib were administered in combination. EGFR-cMET™ ADC was dosed at 2 or 4 MPK.

The combination of EGFR-cMET™ ADC and osimertinib demonstrated improved tumor growth inhibition compared to either agent (EGFR-cMET™ ADC or osimertinib) administered individually.

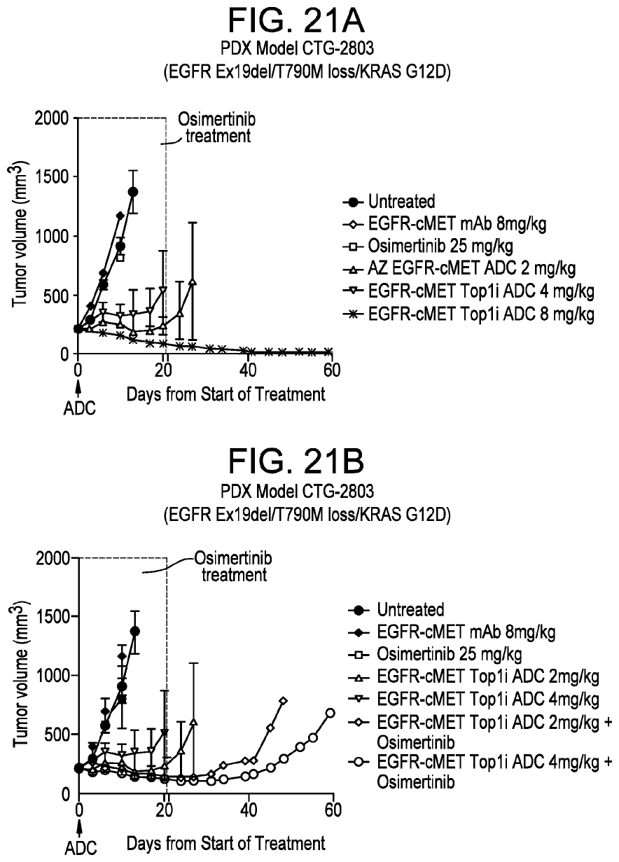

FIG. 21A shows the results for EGFR mutant models ‘CTG-2803’ representing acquired resistance to osimertinib. The EGFR-cMET™ ADC was dosed at 2, 4 and 8 MPK (mg/kg). A group was also dosed with mAb-only control (EGFR-cMET mAb) at 8 mPK or Osimertinib only at 25 MPK. The results show that EGFR-cMET™ ADC monotherapy showed a dose-dependent response. PDX models did not respond to osimertinib or mAb-only monotherapies.

FIG. 21B shows the results for EGFR mutant models ‘CTG-2803’ where the EGFR-cMET™ ADC and osimertinib were administered in combination. EGFR-cMET™ ADC was dosed at 2 or 4 MPK. The combination of EGFR-cMET™ ADC and osimertinib demonstrated improved tumor growth inhibition compared to either agent (EGFR-cMET™ ADC or osimertinib) administered individually.

Together, these results demonstrate that the combination of EGFR-cMET™ ADC and osimertinib showed efficacy across a range of EGFR mutant cancers, including those that are classed as osimertinib resistant.

### Example 11—Response of EGFRmut NSCLC PDX to ADC in Combination with Osimertinib

This example sets to further evaluate the combination efficacy of osimertinib and the EGFR-cMET™ ADC in NSCLC PDX models with mutant EGFR. An additional 23 NSCLC PDX models were enrolled, encompassing a variety of EGFR mutations. Studies were performed largely as set out in the section 5.1 of Example 10.

Mice for each PDX were generally set into three groups, receiving either osimertinib 25 mg/kg daily for 21 days, EGFR-cMET™ ADC 2 mg/kg or osimertinib 25 mg/kg daily and EGFR-cMET™ ADC 2 mg/kg. Experiments were conducted with isotype-ADC R347 controls dosed at 8 mg/kg and/or naked EGFR-cMET™ mAb controls dosed at 8 mg/kg.

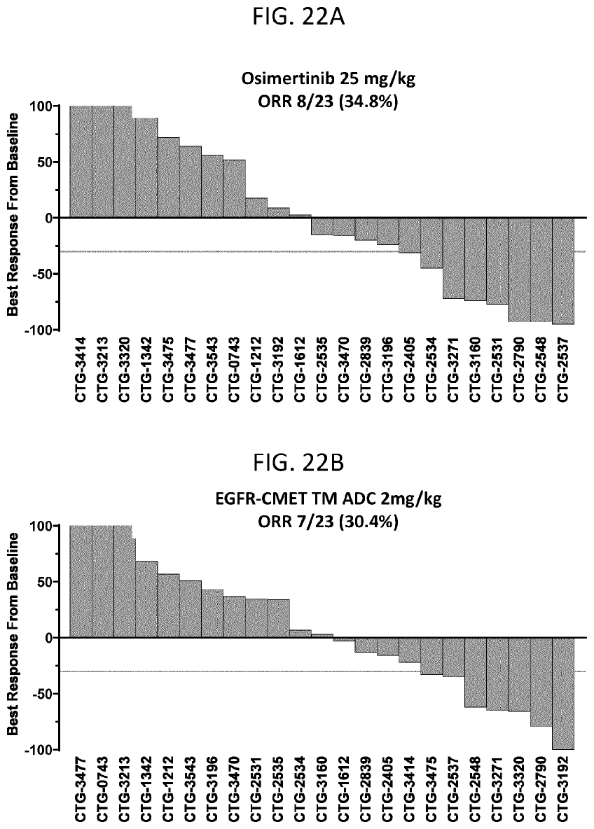

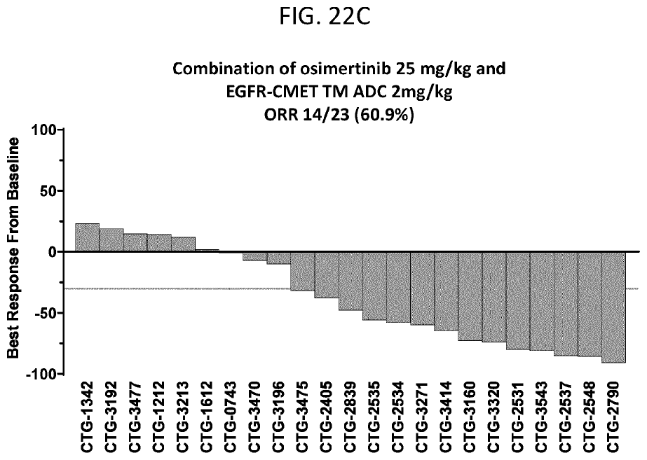

Responses for each of the models for each of the three treatment groups are shown in FIGS. 22A, 22B and 22C.

The results show that responses were observed in 14/23 (61%) models in the combination group, whereas the osimertinib and EGFR-cMET™ ADC monotherapy groups displayed response rates in 8/23 (34.8%) and 7/23 (30.4%) models, respectively. A response is defined as a 30% regression in tumor volume from baseline. Regressions were analysed starting from one week after dosing. The values reported are the best response observed over the duration of the study.

More detailed response data as well as information about the EGFR mutation status is provided in the Table below.

Table 9 shows the best overall response for each study arm for each model R means response, NR means no response. 0 change indicates change in tumor volume compared to baseline

| Model | Osimertinib | Response | ADC | Response | Combo | Response | EGFRmut |
|---|---|---|---|---|---|---|---|
| CTG-0743 | 52% | NR | 140% | NR | −1% | NR | A1158V |
| CTG-1212 | 18% | NR | 57% | NR | 14% | NR | Ex19del, |
| | | | | | | | T790M |
| CTG-1342 | 92% | NR | 68% | NR | 23% | NR | R521K |
| CTG-1612 | 3% | NR | −3% | NR | 2% | NR | R521K |
| CTG-2405 | −31% | R | −16% | NR | −38% | R | L858R, |
| | | | | | | | A289V |
| CTG-2531 | −77% | R | 35% | NR | −80% | R | Ex19del |
| CTG-2534 | −45% | R | 7% | NR | −58% | R | G719C, |
| | | | | | | | S768I |
| CTG-2535 | −15% | NR | 34% | NR | −56% | R | Ex19del |
| CTG-2537 | −95% | R | −35% | R | −85% | R | L858R, |
| | | | | | | | T790M |
| CTG-2548 | −93% | R | −62% | R | −86% | R | L858R, |
| | | | | | | | multiple |
| | | | | | | | uncommon |
| CTG-2790 | −93% | R | −79% | R | −91% | R | Ex19del |
| CTG-2839 | −20% | NR | −13% | NR | −48% | R | Ex19del |
| CTG-3160 | −74% | R | 3% | NR | −73% | R | Ex19del |
| CTG-3192 | 9% | NR | −100% | R | 19% | NR | Ex19del |
| CTG-3196 | −24% | NR | 43% | NR | −10% | NR | L858R |
| CTG-3213 | 118% | NR | 140% | NR | 12% | NR | R1068L |
| CTG-3271 | −72% | R | −65% | R | −60% | R | Ex19del, |
| | | | | | | | T790M |
| CTG-3320 | 102% | NR | −66% | R | −74% | R | Ex19del |
| CTG-3414 | 186% | NR | −22% | NR | −65% | R | Ex19del, |
| | | | | | | | T790M |
| CTG-3470 | −16% | NR | 37% | NR | −7% | NR | Ex19del |
| CTG-3475 | 72% | NR | −33% | R | −32% | R | L858R |
| CTG-3477 | 64% | NR | 184% | NR | 15% | NR | Ex19del, |
| | | | | | | | T790M |
| CTG-3543 | 56% | NR | 51% | NR | −81% | R | Ex19del |

### REFERENCES

A number of publications are cited above in order to more fully describe and disclose the disclosure and the state of the art to which the disclosure pertains. Full citations for these references are provided below. The entirety of each of these references is incorporated herein.

| | |
|---|---|
| Angevin, 2017 | Angevin, E., et al., Phase I study of ABBV-399, a c-Met antibody-drug conjugate |
| | (ADC), as monotherapy and in combination with erlotinib in patients (pts) with |
| | non-small cell lung cancer (NSCLC). Journal of Clinical Oncology, 2017. |
| | 35(15_suppl): p. 2509-2509. |
| Bagshawe, 1991 | Bagshawe et al. Antibody-enzyme conjugates can generate cytotoxic drugs from |
| | inactive precursors at tumor sites. Antibody, Immunoconjugates and |
| | Radiopharmaceuticals, 1991, 4: 915-922 |
| Bardia, 2017 | Bardia et al. Efficacy and Safety of Anti-Trop-2 Antibody Drug Conjugate |
| | Sacituzumab Govitecan (IMMU-132) in Heavily Pretreated Patients With |
| | Metastatic Triple-Negative Breast Cancer. J Clin Oncol. 2017, 35(19): 2141-2148 |
| Beck, 2017 | Beck, A., et al. Strategies and challenges for the next generation of antibody-drug |
| | conjugates., Nature Reviews Drug Discovery, 2017, 16, pp. 315-337 |
| Birchmeier, | Birchmeier, C., et al., Met, metastasis, motility and more. Nat Rev Mol Cell Biol, |
| 2003 | 2003. 4: p. 915-925. |
| Brinkmann, | Brinkmann, U. and R. E. Kontermann, The making of bispecific antibodies. MAbs, |
| 2017 | 2017. 9(2): p. 182-212. |
| Calvo, 2017 | Calvo, E., et al., Preliminary results from a phase 1 study of the antibody-drug |
| | conjugate ABBV-221 in patients with solid tumors likely to express EGFR. Journal |
| | of Clinical Oncology, 2017. 35(15_suppl): p. 2510-2510. |
| Cardillo, 2015 | Cardillo et al. Sacituzumab Govitecan (IMMU-132), an Anti-Trop-2/SN-38 |
| | Antibody-Drug Conjugate: Characterization and Efficacy in Pancreatic, Gastric, |
| | and Other Cancers. Bioconjug Chem., 2015, 26(5): 919-31 |
| Chalouni, 2018 | Chalouni and Doll 2018 J Exp Clin Cancer Res. 37(1): 20. |
| Chang 2020 | Chang et al. The Clinical Significance of RAS, PIK3CA, and PTEN Mutations in |
| | Non-Small Cell Lung Cancer Using Cell-Free DNA J Clin Med 2020 9(8): 2642 |
| Comer, 2018 | Comer, F., C. Gao, and S. Coats, Bispecific and Biparatopic Antibody Drug |
| | Conjugates, in Innovations for Next-Generation Antibody-Drug Conjugates, M. |
| | Damelin, Editor. 2018, Springer International Publishing: Cham. p. 267-280. |
| Dimasi, 2017 | Dimasi et al. Efficient Preparation of Site-Specific Antibody-Drug Conjugates |
| | Using Cysteine Insertion. Mol Pharmaceuticals, 2017, 14(5) 1501-1516 |
| Domling, 2005 | Domling et al. Myxobacterial epothilones and tubulysins as promising anticancer |
| | agents. Mol. Diversity, 2005 9: 141-147 |
| Dornan, 2009 | Dornan et al., Therapeutic potential of an anti-CD79b antibody-drug conjugate, |
| | anti-CD79b-vc-MMAE, for the treatment of non-Hodgkin lymphoma. Blood, 2009, |
| | 114(13): 2721-2729 |
| Dubowchik, | Dubowchik et al., Cathepsin B-labile dipeptide linkers for lysosomal release of |
| 2002 | doxorubicin from internalizing immunoconjugates: model studies of enzymatic |
| | drug release and antigen-specific in vitro anticancer activity, Bioconjugate |
| | Chemistry, 2002, 13, 855-869 |
| Engelman, 2007 | Engelman, J.A., et al., MET amplification leads to gefitinib resistance in lung |
| | cancer by activating ERBB3 signaling. Science, 2007. 316: p. 1039-1043. |
| Ferguson, 2008 | Ferguson, Annu Rev Biophys. (2008) 37: 353-373. |
| Foot 1992 | Foot and Winter Antibody framework residues affecting the conformation of the |
| | hypervariable loops J Mol Biol. 1992 224(2): 487-99 |
| Gébleux, 2016 | Gebleux and Casi, Pharmacol Ther (2016) 167: 48-59 |
| Graziano, 2004 | Graziano and Guptill, Methods Mol Biol. 2004; 283: 71-85. |
| Hamblett, 2004 | Hamblett et al., Effects of drug loading on the antitumor activity of a monoclonal |
| | antibody drug conjugate. Clin. Cancer Res., 2004, 10: 7063-7070 |
| Hay, 2016 | Hay et al., Targeting CD73 in the tumor microenvironment with MEDI9447. |
| | Oncoimmunology, 2016 5(8): e1208875. |
| Holliger, 2005 | Holliger & Hudson, Nature Biotechnology 23(9): 1126-1136 (2005). |
| Junutula, 2008 | Junutula, et al., Site-specific conjugation of a cytotoxic drug to an antibody |
| | improves the therapeutic index. Nature Biotech., 2008, 26(8): 925-932 |
| Kabat, 1991 | Kabat, E. A. et al. Sequences of Proteins of Immunological Interest, 5th Ed. Public |
| | Health Service, National Institutes of Health, Bethesda, MD, 1991 |
| Klein, 2012 | Klein et al. Progress in overcoming the chain association issue in bispecific |
| | heterodimeric IgG antibodies. MAbs, 2012, 4: 653-63 |
| Kontermann, | Kontermann, MAbs 2012, 4(2): 182-197 |
| 2012 | |
| Kontermann, | Kontermann and Brinkmann 2015, 20(7): 838-847 |
| 2015 | |
| Kunkel 1985 | Kunkel. Rapid and efficient site-specific mutagenesis without phenotypic selection |
| | Proc Natl Acad Sci U S A. 1985 82(2): 488-92. |
| Ledermann, | Ledermann et al. A phase-I study of repeated therapy with radiolabelled antibody |
| 1991 | to carcinoembryonic antigen using intermittent or continuous administration of |
| | cyclosporin A to suppress the immune response. Int. J. Cancer, 1991, 47: 659-664 |
| Mazor, 2015 | Mazor, Y., et al., Insights into the molecular basis of a bispecific antibody's target |
| | selectivity. MAbs, 2015. 7(3): p. 461-9. |
| Mazor, 2017 | Mazor, Y., et al., Enhanced tumor-targeting selectivity by modulating bispecific |
| | antibody binding affinity and format valence. Sci Rep, 2017. 7: p. 40098. |
| McDermott, | McDermott, U., et al., Acquired Resistance of Non-Small Cell Lung Cancer Cells |
| 2010 | to MET Kinase Inhibition Is Mediated by a Switch to Epidermal Growth Factor |
| | Receptor Dependency. Cancer research, 2010. 70(4): p. 1625-1634. |
| Mo, 2017 | Mo, H. N. and P. Liu, Targeting MET in cancer therapy. Chronic Dis Transl Med, |
| | 2017. 3(3): p. 148-153. |
| Moores, 2016 | Moores, S. L., et al., A Novel Bispecific Antibody Targeting EGFR and cMet that |
| | is Effective Against EGFR Inhibitor-Resistant Lung Tumors. Cancer research, |
| | 2016. |
| Ogitani, 2016a | Ogitani et al. Wide application of a novel topoisomerase I inhibitor-based drug |
| | conjugation technology. Bioorg Med Chem Lett. 2016, 26(20): 5069-5072 |
| Ogitani, 2016b | Ogitani et al. DS-8201a, A Novel HER2-Targeting ADC with a Novel DNA |
| | Topoisomerase I Inhibitor, Demonstrates a Promising Antitumor Efficacy with |
| | Differentiation from T-DM1. Clin Cancer Res. 2016, 22(20): 5097-5108 |
| Organ, 2011 | Organ, S. L. and M. S. Tsao, An overview of the c-MET signaling pathway. |
| | Therapeutic advances in medical oncology, 2011. 3(1 Suppl): p. S7-S19. |
| Organesyan, | Organesyan et al. Structural characterization of a human Fc fragment engineered |
| 2008 | for lack of effector functions. Acta Crystallogr D Biol Crystallogr. 2008; 64(Pt |
| | 6): 700-4. |
| Philips, 2018 | Phillips, A. C., et al., Characterization of ABBV-221, a Tumor-Selective EGFR |
| | Targeting Antibody Drug Conjugate. Mol Cancer Ther, 2018. |
| Persic, 1997 | Persic et al. An integrated vector system for the eukaryotic expression of antibodies |
| | or their fragments after selection from phage display libraries. Gene, 1997, 187: |
| | 9-18. |
| Ridgway, 1996 | Ridgway et al., ‘Knobs-into-holes’ engineering of antibody CH3 domains for heavy |
| | chain heterodimerization. Protein Eng., 1996, 9: 617-621 |
| Sanderson, 2005 | Sanderson et al., In vivo drug-linker stability of an anti-CD30 dipeptide-linked |
| | auristatin immunoconjugate. Clin. Cancer Res. 2005, 11: 843-852 |
| Sierra, 2011 | Sierra, J. R. and M.-S. Tsao, c-MET as a potential therapeutic target and biomarker |
| | in cancer. Therapeutic Advances in Medical Oncology, 2011. 3(1 suppl): p. S21- |
| | S35. |
| Suda 2009: | Suda, K. et al. EGFR T790M mutation: a double role in lung cancer cell survival? |
| | J Thorac Oncol. 2009; 4(1): 1-4. |
| Suda, 2010 | Suda, K., et al., Reciprocal and complementary role of MET amplification and |
| | EGFR T790M mutation in acquired resistance to kinase inhibitors in lung cancer. |
| | Clinical cancer research: an official journal of the American Association for |
| | Cancer Research, 2010. 16(22): p. 5489-5498. |
| Tolcher, 2014 | Tolcher, A., et al., 342 A phase 1/2 study evaluating the safety, pharmacokinetics |
| | and efficacy of ABT-414 in subjects with advanced solid tumors likely to over- |
| | express the epidermal growth factor receptor (EGFR). European Journal of |
| | Cancer, 2014. 50: p. 111. |
| Vaughan, 1996 | Vaughan, et al., Human antibodies with sub-nanomolar affinities isolated from a |
| | large non-immunized phage display library, Nat Biotechnol,. 1996. 14: p. 309 |
| Vyse and Huang, | Vyse, S and Huang, P. Targeting EGFR exon 20 insertion mutations in non-small |
| 2019 | cell lung cancer. Signal Transduction and Targeted Therapy 2019 4(5) |
| Wang, 2017 | Wang, J., et al., ABBV-399, a c-Met Antibody-Drug Conjugate that Targets Both |
| | MET-Amplified and c-Met-Overexpressing Tumors, Irrespective of MET Pathway |
| | Dependence. Clinical cancer research: an official journal of the American |
| | Association for Cancer Research, 2017. 23(4): p. 992-1000. |
| Wang, 2018 | Wang, X. et al., IgG Fc engineering to modulate antibody effector functions Protein |
| | Cell. 2018; 9(1): 63-73 |

Sequences
CDR sequences for low affinity anti-EGFR binding arm (RAA22)
HCDR1-DNDFS (SEQ ID NO: 1)
HCDR2-AIVAVFRTETYAQKFQD (SEQ ID NO: 2)
HCDR3-RLMSAISGPGAPLLM (SEQ ID NO: 3)
LCDR1-TGTSSDVGGYNYVS (SEQ ID NO: 4)
LCDR2-DVSKRPS (SEQ ID NO: 5)
LCDR3-SSYTSSDTLEI (SEQ ID NO: 6)
CDR sequences for high affinity anti-EGFR binding arm (QD6)
HCDR1-DNDFS (SEQ ID NO: 1)
HCDR2-AIVAVVRTETYAQKFQD (SEQ ID NO: 7)
HCDR3-RLMSAISGPGAPLLM (SEQ ID NO: 3)
LCDR1-TGTSSDVGGYNYVS (SEQ ID NO: 4)
LCDR2-DVSERPS (SEQ ID NO: 66)
LCDR3-FSYTSSDTLEI (SEQ ID NO: 67)
FR sequences for anti-EGFR binding arms RAA22 and QD6
HFR1-QVQLVQSGAEVKKPGSSVKVSCKASGGTFS (SEQ ID NO: 8)
HFR2-WVRQAPGQGLEWMG (SEQ ID NO: 9)
HFR3-RVKITADISTRTTYMELSSLRSEDTAVYYCAR (SEQ ID NO: 10)
HFR4-WGQGTLVTVSS (SEQ ID NO: 11)
LFR1-QSALTQPRSVSGSPGQSVTISC (SEQ ID NO: 12)
LFR2-WYQQHPGKAPKLMIY (SEQ ID NO: 13)
LFR3-GVPDRFSGSKSGNTASLTISGLQAEDEADYYC (SEQ ID NO: 14)
LFR4-FGGGTKLTVL (SEQ ID NO: 15)
Amino acid sequence of the variable heavy (VH) region of low affinity anti-EGFR binding arm
(RAA22) (SEQ ID NO: 16)
QVQLVQSGAEVKKPGSSVKVSCKASGGTFSDNDFSWVRQAPGQGLEWMGAIVAVFRTETYAQKFQDRVKITAD
ISTRTTYMELSSLRSEDTAVYYCARRLMSAISGPGAPLLMWGQGTLVTVSS
Nucleic acid sequence of the VH region of low affinity anti-EGFR binding arm (RAA22) (SEQ ID
NO: 17):
CAGGTGCAGCTGGTGCAGTCTGGGGCCGAAGTGAAGAAACCCGGCAGCAGCGTGAAGGTGTCCTGTAAAGC
CAGCGGCGGCACCTTCAGCGACAACGACTTTAGCTGGGTCCGACAGGCCCCTGGACAGGGCCTGGAATGGA
TGGGAGCCATCGTGGCCGTGTTCCGGACAGAGACATACGCCCAGAAATTCCAGGACAGAGTGAAAATCACC
GCCGACATCAGCACCAGAACCACCTACATGGAACTGAGCAGCCTGAGAAGCGAGGACACCGCCGTGTACTA
CTGCGCCAGACGGCTGATGTCTGCCATCTCTGGACCTGGCGCTCCTCTGCTCATGTGGGGACAGGGAACACT
GGTCACCGTGTCCAGC
Amino acid sequence of the VH region of anti-EGFR antibody clone QD6 (SEQ ID NO: 18):
QVQLVQSGAEVKKPGSSVKVSCKASGGTFSDNDFSWVRQAPGQGLEWMGAIVAVVRTETYAQKFQDRVKITA
DISTRTTYMELSSLRSEDTAVYYCARRLMSAISGPGAPLLMWGQGTLVTVSS
Nucleic acid sequence of the VH region of anti-EGFR antibody clone QD6 (SEQ ID NO: 19):
CAGGTGCAGCTGGTGCAGTCTGGGGCCGAAGTGAAGAAACCCGGCAGCAGCGTGAAGGTGTCCTGTAAAGC
CAGCGGCGGCACCTTCAGCGACAACGACTTTAGCTGGGTCCGACAGGCCCCTGGACAGGGCCTGGAATGGA
TGGGAGCCATCGTGGCCGTGGTCCGGACAGAGACATACGCCCAGAAATTCCAGGACAGAGTGAAAATCACC
GCCGACATCAGCACCAGAACCACCTACATGGAACTGAGCAGCCTGAGAAGCGAGGACACCGCCGTGTACTA
CTGCGCCAGACGGCTGATGTCTGCCATCTCTGGACCTGGCGCTCCTCTGCTCATGTGGGGACAGGGAACACT
GGTCACCGTGTCCAGC
Amino acid sequence of the variable light (VL) region of anti-EGFR antibody clone RAA22 (SEQ
ID NO: 20):
QSALTQPRSVSGSPGQSVTISCTGTSSDVGGYNYVSWYQQHPGKAPKLMIYDVSKRPSGVPDRFSGSKSGNTASL
TISGLQAEDEADYYCSSYTSSDTLEIFGGGTKLTVL
Nucleic acid sequence of the VL region of anti-EGFR antibody clone RAA22 (SEQ ID NO: 21):
CAGTCTGCCCTGACTCAGCCTCGCTCAGTGTCCGGGTCTCCTGGACAGTCAGTCACCATCTCCTGCACTGGA
ACCAGCAGTGATGTTGGTGGTTATAACTATGTCTCCTGGTACCAACAGCACCCAGGCAAAGCCCCCAAACTC
ATGATTTATGATGTCAGTAAGCGGCCCTCAGGGGTCCCTGATCGCTTCTCTGGCTCCAAGTCTGGCAACACG
GCCTCCCTGACCATCTCTGGGCTCCAGGCTGAGGACGAGGCTGATTATTACTGCAGTTCATATACAAGCAGC
GACACTCTCGAAATATTCGGCGGAGGGACCAAGCTGACCGTCCTA
Amino acid sequence of the VL region of high affinity anti-EGFR binding arm (QD6) (SEQ ID
NO: 22):
QSALTQPRSVSGSPGQSVTISCTGTSSDVGGYNYVSWYQQHPGKAPKLMIYDVSERPSGVPDRFSGSKSGNTASL
TISGLQAEDEADYYCFSYTSSDTLEIFGGGTKLTVL
Nucleic acid sequence of the VL region of high affinity anti-EGFR binding arm (QD6) (SEQ ID
NO: 23):
CAGTCTGCCCTGACTCAGCCTCGCTCAGTGTCCGGGTCTCCTGGACAGTCAGTCACCATCTCCTGCACTGGA
ACCAGCAGTGATGTTGGTGGTTATAACTATGTCTCCTGGTACCAACAGCACCCAGGCAAAGCCCCCAAACTC
ATGATTTATGATGTCAGTGAACGGCCCTCAGGGGTCCCTGATCGCTTCTCTGGCTCCAAGTCTGGCAACACG
GCCTCCCTGACCATCTCTGGGCTCCAGGCTGAGGACGAGGCTGATTATTACTGCTTCTCATATACAAGCAGC
GACACTCTCGAAATATTCGGCGGAGGGACCAAGCTGACCGTCCTA
CDR sequences for anti-cMet binding arm B09-GL
HCDR1-DYYIH (SEQ ID NO: 24)
HCDR2-WMNPNSGNTGYAQKFQG (SEQ ID NO: 25)
HCDR3-GQGYTHS (SEQ ID NO: 26)
LCDR1-RASEGIYHWLA (SEQ ID NO: 27)
LCDR2-KASSLAS (SEQ ID NO: 28)
LCDR3-QQYSNYPPT (SEQ ID NO: 29)
FR sequences for anti-cMet binding arm B09-GL
HFR1-QVQLVQSGAEVKKPGASVKVSCKASGYTFT (SEQ ID NO: 30)
HFR2-WVRQATGQGLEWMG (SEQ ID NO: 31)
HFR3-RVTMTRDTSISTAYMELSSLRSEDTAVYYCAR (SEQ ID NO: 32)
HFR4-WGQGTMVTVSS (SEQ ID NO: 33)
LFR1-DIQMTQSPSTLSASVGDRVTITC (SEQ ID NO: 34)
LFR2-WYQQKPGKAPKLLIY (SEQ ID NO: 35)
LFR3-GVPSRFSGSGSGTEFTLTISSLQPDDFATYYC (SEQ ID NO: 36)
LFR4-FGGGTKLEIK (SEQ ID NO: 37)
Amino acid sequence of the variable heavy (VH) region of anti-cMet binding arm B09-GL (SEQ ID
NO: 38):
QVQLVQSGAEVKKPGASVKVSCKASGYTFTDYYIHWVRQATGQGLEWMGWMNPNSGNTGYAQKFQGRVTM
TRDTSISTAYMELSSLRSEDTAVYYCARGQGYTHSWGQGTMVTVSS
Nucleic acid sequence of the VH region of anti-cMet binding arm B09-GL (SEQ ID NO: 39):
CAAGTGCAGCTGGTGCAGTCTGGCGCCGAAGTGAAGAAACCTGGCGCCAGCGTGAAGGTCAGCTGCAAGGC
CAGCGGCTACACCTTCACCGACTACTACATCCACTGGGTCCGCCAGGCCACAGGCCAGGGACTGGAATGGA
TGGGCTGGATGAACCCCAACAGCGGCAACACCGGCTACGCCCAGAAATTCCAGGGCAGAGTGACCATGACC
CGGGACACCAGCATCAGCACCGCCTACATGGAACTGAGCAGCCTGCGGAGCGAGGACACCGCCGTGTACTA
CTGTGCCAGAGGCCAGGGCTACACCCACAGCTGGGGCCAGGGCACCATGGTCACAGTGTCCAGC
Amino acid sequence of the variable light (VL) region of anti-cMet binding arm B09-GL (SEQ ID
NO: 40):
DIQMTQSPSTLSASVGDRVTITCRASEGIYHWLAWYQQKPGKAPKLLIYKASSLASGVPSRFSGSGSGTEFTLTISS
LQPDDFATYYCQQYSNYPPTFGGGTKLEIK
Nucleic acid sequence of the VL region of anti-cMet binding arm B09-GL (SEQ ID NO: 41):
GACATCCAGATGACCCAGAGCCCCAGCACCCTGAGCGCCAGCGTCGGCGACAGAGTGACCATCACCTGTCG
GGCCAGCGAGGGCATCTACCACTGGCTGGCCTGGTATCAGCAGAAGCCCGGCAAGGCCCCCAAGCTGCTGA
TCTACAAGGCCAGCAGCCTGGCCAGCGGAGTCCCTAGCAGATTTTCTGGCAGCGGCAGCGGCACCGAGTTC
ACCCTGACCATCAGCAGCCTGCAGCCCGACGACTTCGCCACCTACTACTGCCAGCAGTACAGCAACTACCCC
CCCACCTTCGGCGGAGGCACCAAGCTGGAAATCAAG
Amino acid sequence of a human immunoglobulin G1 heavy chain constant (CH) region (SEQ ID NO:
42):
ASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRW
QQGNVFSCSVMHEALHNHYTQKSLSLSPGK

Amino acid sequence of a human immunoglobulin G1 CH region modified to include “Knob” mutation, interchain cysteine mutations, a cysteine to form a stabilizing disulfide bridge and with a cysteine insertion (SEQ ID NO: 43): Following substitutions are underlined:

“Knob” mutation (T366W); interchain cysteine mutations (F126C and C219V); stabilizing cysteine mutation (S354C); and cysteine insertion (C239i), where numbering of residues is according to EU index.

ASTKGPSVCPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSVDKTHTCPPCPAPELLGGPSCVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKN
QVSLWCLVKGFYPSDIAVEWESNGQPENNYKTTPPVLDSDGSFFLYSKL
TVDKSRWQQGNVFSCSVMHEALHNHYTQKSLSLSPG

Amino acid sequence of a human immunoglobulin G1 CH region modified to include “Knob” mutation, interchain cysteine mutations, a cysteine to form a stabilizing disulfide bridge and without a cysteine insertion (SEQ ID NO: 44): Following substitutions are underlined:

“Knob” mutation (T366W); interchain cysteine mutations (F126C and C219V); and stabilizing cysteine mutation (S354C), where numbering of residues is according to EU index.

ASTKGPSVCPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSVDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPCREEMTKNQ
VSLWCLVKGFYPSDIAVEWESNGQPENNYKTTPPVLDSDGSFFLYSKLT
VDKSRWQQGNVFSCSVMHEALHNHYTQKSLSLSPG

Amino acid sequence of a human immunoglobulin G1 CH region modified to include “Hole” mutations, a cysteine to form a stabilizing disulfide bridge and with a cysteine insertion (SEQ ID NO: 45): Following substitutions are underlined:

“Hole” mutations (T366S, L368A, and Y407V); stabilizing cysteine mutation (Y349C); and cysteine insertion (C239i), where numbering of residues is according to EU index.

ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCDKTHTCPPCPAPELLGGPSCVFLFPPKPKDTL
MISRTPEVTCVVVDVSHEDPEVKFNWYVDGVEVHNAKTKPREEQY
NSTYRVVSVLTVLHQDWLNGKEYKCKVSNKALPAPIEKTISKAKG
QPREPQVCTLPPSREEMTKNQVSLSCAVKGFYPSDIAVEWESNGQ
PENNYKTTPPVLDSDGSFFLVSKLTVDKSRWQQGNVFSCSVMHEA
LHNHYTQKSLSLSPGK

Amino acid sequence of a human immunoglobulin G1 CH region modified to include “Hole” mutations, a cysteine to form a stabilizing disulfide bridge and without a cysteine insertion (SEQ ID NO: 46): Following substitutions are underlined:

“Hole” mutations (T366S, L368A, and Y407V); and stabilizing cysteine mutation (Y349C), where numbering of residues is according to EU index.

ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVCTLPPSREEMTKNQVSLSCAVKGFYPSDIAVEWESNGQP
ENNYKTTPPVLDSDGSFFLVSKLTVDKSRWQQGNVFSCSVMHEAL
HNHYTQKSLSLSPGK
Amino acid sequence of a wild-type human
immunoglobulin kappa constant region
(SEQ ID NO: 47):
RTVAAPSVFIFPPSDEQLKSGTASVVCLLNNFYPREAKQWKDNAL
QSGNSQESVTEQDSKDSTYSLSSTLTLSKADYEKHKVYACEVTHQ
GLSSPVTKSFNRGEC

Amino acid sequence of a human immunoglobulin kappa constant region modified to include S121C and C214V substitutions (SEQ ID NO: 48):

Following substitutions are underlined:

S121C and C214V, wherein numbering is according to EU index PGP-161 DNA

RTVAAPSVFIFPPCDEQLKSGTASVVCLLNNFYPREAKVQWKVDN
ALQSGNSQESVTEQDSKDSTYSLSSTLTLSKADYEKHKVYACEVT
HQGLSSPVTKSFNRGEV
Amino acid sequence of a human immunoglobulin
lambda constant region (SEQ ID NO: 49):
GQPKAAPSVTLFPPSSEELQANKATLCLISDFYPGATVAWKADSS
PVKAGVETTTPSKQSNNKYAASSYLSLTPEQWKSHRSYSCQVTHE
GSTVEKTVAPTECS

Amino acid sequence of the heavy chain of anti-cMet binding arm B09-GL with cysteine insertion (SEQ ID NO: 50): Following substitutions are underlined:

“Knob” mutation (T366W); interchain cysteine mutations (F126C and C219V); stabilizing cysteine mutation (S354C); and cysteine insertion (C239i), where numbering of residues is according to EU index.

QVQLVQSGAEVKKPGASVKVSCKASGYTFTDYYIHWVRQATGQGL
EWMGWMNPNSGNTGYAQKFQGRVTMTRDTSISTAYMELSSLRSED
TAYYCARGQGYTHSWGQGTMVTVSSASTKGPSVCPLAPSSKSTSG
GTAALGCLVKDYFPEPVTVSWNSGALTSGVHTFPALQSSGLYSLS
SVVTVPSSSLGTQTYICNVNHKPSNTKDKRVEPKSVDKTHTCPPC
PAPELLGGPSCVFLFPPKPKDTLMISRTPEVTCVVVDVSHEDPEV
KFNWYVDGVEVHNAKTKPREEQYNSTYRVVSVLTVLHQDWLNGKE
YKCKVSNKALPAPIEKTISKAKGQPREPQVYTLPPCREEMTKNQV
SLWCLVKGFYPSDIAEWESNGQPENNYKTTPPVLDSDGSFFLYSK
LTVDKSRWQQGNVFSCSVMHEALHNHYTQKSLSLSPG

Amino acid sequence of the heavy chain of anti-cMet binding arm B09-GL without cysteine insertion (SEQ ID NO: 51): Following substitutions are underlined:

“Knob” mutation (T366W); interchain cysteine mutations (F126C and C219V); and stabilizing cysteine mutation (S354C), where numbering of residues is according to EU index.

QVQLVQSGAEVKKPGASVKVSCKASGYTFTDYYIHWVRQATGQGL
EWMGWMNPNSGNTGYAQKFQGRVTMTRDTSISTAYMELSSLRSED
TAYYCARGQGYTHSWGQGTMVTVSSASTKGPSVCPLAPSSKSTSG
GTAALGCLVKDYFPEPVTVSWNSGALTSGVHTFPALQSSGLYSLS
SVVTVPSSSLGTQTYICNVNHKPSNTKVDKREPKSVDKTHTCPPC
PAPELLGGPSVFLFPPKPKDTLMISRTPEVTCVVVDVSHEDPEVK
FNWYVDGVEVHNAKTKPREEQYNSTYRVVSVLTVLHQDWLNGKEY
KCKVSNKALPAPIEKTISKAKGQPREPQVYTLPPCREEMTKNQVS
LWCLVKGFYPSDIAEWESNGQPENNYKTTPPVLDSDGSFFLYSKL
TVDKSRWQQGNVFSCSVMHEALHNHYTQKSLSLSPG

Amino acid sequence of the light chain of anti-cMet binding arm B09-GL (SEQ ID NO: 52): Following substitutions are underlined: S121C and C214V, wherein numbering is according to EU index

DIQMTQSPSTLSASVGDRVTITCRASEGIYHWLAWYQQKPGKAPK
LLIYKASSLASGVPSRFSGSGSGTEFTLTISSLQPDDFATYYCQQ
YSNYPPTFGGGTKLEIKRTVAAPSVFIFPPCDEQLKSGTASVVCL
LNNFYPREAKVQWKVDNALQSGNSQESVTEQDSKDSTYSLSSTLT
LSKADYEKHKVYACEVTHQGLSSPVTKSFNRGEV

Amino acid sequence of the heavy chain of high affinity anti-EGFR binding arm (QD6) with cysteine insertion (SEQ ID NO: 53):

Following substitutions are underlined:

“Hole” mutations (T366S, L368A, and Y407V); stabilizing cysteine mutation (Y349C); and cysteine insertion (C239i), where numbering of residues is according to EU index.

QVQLVQSGAEVKKPGSSVKVSCKASGGTFSDNDFSWVRQAPGQGL
EWMGAIVAVRTETYAQKFQDRVKITADISTRTTYMELSSLRSEDT
AYYCARRLMSAISGPGAPLLMWGQGTLVTVSSASTKGPSVFPLAP
SSKSTSGGTAALGCLKDYFPEPVTVSWNSGALTSGVHTFPALQSS
GLYSLSSVVTVPSSSLGTQTYICNVNHKPSNTKVDKRVEPKSCDK
THTCPPCPAPELLGGPSCVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVCTLPPSRE
EMTKNQVSLSCAVKGFYPSDIAEWESNGQPENNYKTTPPVLDSDG
SFFLVSKLTVDKSRWQQGNVFSCSVMHEALHNHYTQKSLSLSPGK

Amino acid sequence of the heavy chain of high affinity anti-EGFR binding arm (QD6) without cysteine insertion (SEQ ID NO: 54):

Following substitutions are underlined:

“Hole” mutations (T366S, L368A, and Y407V); and stabilizing cysteine mutation (Y349C), where numbering of residues is according to EU index.

QVQLVQSGAEVKKPGSSVKVSCKASGGTFSDNDFSWVRQAPGQGL
EWMGAIVAVRTETYAQKFQDRVKITADISTRTTYMELSSLRSEDT
AYYCARRLMSAISGPGAPLLMWGQGTLVTVSSASTKGPSVFPLAP
SSKSTSGGTAALGCLKDYFPEPVTVSWNSGALTSGVHTFPALQSS
GLYSLSSVVTVPSSSLGTQTYICNVNHKPSNTKVDKRVEPKSCDK
THTCPPCPAPELLGGPSVFLFPPKPKDTLMISRTPEVTCVVVDVS
HEDPEVKFNWYVDGEVHNAKTKPREEQYNSTYRVVSVLTVLHQDW
LNGKEYKCKVSNKALPAPIEKTISKAKGQPREPQVCTLPPSREEM
TKNQVSLSCAVKGFYPSDIAEWESNGQPENNYKTTPPVLDSDGSF
FLVSKLTVDKSRWQQGNVFSCSVMHEALHNHYTQKSLSLSPGK
Amino acid sequence of the light chain of
high affinity anti-EGFR binding arm (QD6)
(SEQ ID NO: 55):
QSALTQPRSVSGSPGQSVTISCTGTSSDVGGYNYVSWYQQHPGKA
PKLMIYDVSERPSGVPDRFSGSKSGNTASLTISGLQAEDEADYYC
FSYTSSDTLEIFGGGTKLTVLGQPKAAPSVTLFPPSSEELQANKA
TLCLISDFYPGATVAWKADSSPVKAGVETTTPSKQSNNKYAASSY
LSLTPEQWKSHRSYSCQVTHEGSTVEKTVAPTECS

Amino acid sequence of the heavy chain of low affinity anti-EGFR binding arm (RAA22) with cysteine insertion (SEQ ID NO: 56):

Following substitutions are underlined:

“Hole” mutations (T366S, L368A, and Y407V); stabilizing cysteine mutation (Y349C); and cysteine insertion (C239i), where numbering of residues is according to EU index.

QVQLVQSGAEVKKPGSSVKVSCKASGGTFSDNDFSWVRQAPGQGL
EWMGAIVAFRTETYAQKFQDRVKITADISTRTTYMELSSLRSEDT
AYYCARRLMSAISGPGAPLLMWGQGTLVTVSSASTKGPSVFPLAP
SSKSTSGGTAALGCLVKDYFPEPVTVSWNSGALTSGVHTFPALQS
SGLYSLSSVVTVPSSSLGTQTYICNVNHKPSNTKVDKRVEPKSCD
KTHTCPPCPAPELLGGPSCVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLSCAVKGFYPSDIAEWESNGQPENNYKTTPPVLDSD
GSFFLVSKLTVDKSRWQQGNVFSCSVMHEALHNHYTQKSLSLSPG
K

Amino acid sequence of the heavy chain of low affinity anti-EGFR binding arm (RAA22) without cysteine insertion (SEQ ID NO: 57):

Following substitutions are underlined:

“Hole” mutations (T366S, L368A, and Y407V); and stabilizing cysteine mutation (Y349C), where numbering of residues is according to EU index.

QVQLVQSGAEVKKPGSSVKVSCKASGGTFSDNDFSWVRQAPGQGL
EWMGAIVAFRTETYAQKFQDRVKITADISTRTTYMELSSLRSEDT
AYYCARRLMSAISGPGAPLLMWGQGTLVTVSSASTKGPSVFPLAP
SSKSTSGGTAALGCLVKDYFPEPVTVSWNSGALTSGVHTFPALQS
SGLYSLSSVVTVPSSSLGTQTYICNVNHKPSNTKVDKRVEPKSCD
KTHTCPPCPAPELLGGPSCVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLSCAVKGFYPSDIAEWESNGQPENNYKTTPPVLDSD
GSFFLVSKLTVDKSRWQQGNVFSCSVMHEALHNHYTQKSLSLSPG
K
Amino acid sequence of the light chain of
low affinity anti-EGFR binding arm (RAA22)
(SEQ ID NO: 58):
QSALTQPRSVSGSPGQSVTISCTGTSSDVGGYNYVSWYQQHPGKA
PKLMIYDVSKRPSGVPDRFSGSKSGNTASLTISGLQAEDEADYYC
SSYTSSDTLEIFGGGTKLTVLGQPKAAPSVTLFPPSSEELQANKA
TLCLISDFYPGATVAWKADSSPVKAGVETTTPSKQSNNKYAASSY
LSLTPEQWKSHRSYSCQVTHEGSTVEKTVAPTECS

Amino acid sequence of the heavy chain of low affinity anti-EGFR binding arm (RAA22) with triple Mutation™ (SEQ ID NO: 59):

Following substitutions are underlined:

Triple mutation (TM; L234F, L235E and P331S); “Knob” mutation (T366W); interchain cysteine mutations (F126C and C219V); stabilizing cysteine mutation (S354C), where numbering of residues is according to EU index.

QVQLVQSGAEVKKPGSSVKVSCKASGGTFSDNDFSWVRQAPGQGL
EWMGAIVAFRTETYAQKFQDRVKITADISTRTTYMELSSLRSEDT
AYYCARRLMSAISGPGAPLLMWGQGTLVTVSSASTKGPSVCPLAP
SSKSTSGGTAALGCLKDYFPEPVTVSWNSGALTSGVHTFPALQSS
GLYSLSSVVTVPSSSLGTQTYICNVNHKPSNTKVDKRVEPKSVDK
THTCPPCPAPEFEGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPASIEKTISKAKGQPREPQVYTLPPCREE
MTKNQVSLWCLVKGFYPSDIAEWESNGQPENNYKTTPPVLDSDGS
FFLYSKLTVDKSRWQQGNVFSCSVMHEALHNHYTQKSLSLSPGK

Amino acid sequence of the heavy chain of anti-cMet binding arm with triple Mutation™ (SEQ ID NO: 60): Following substitutions are underlined:

Triple mutation (TM; L234F, L235E and P331S); “Hole” mutations (T366S, L368A, and Y407V); and stabilizing cysteine mutation (Y349C), where numbering of residues is according to EU index.

QVQLVQSGAEVKKPGASVKVSCKASGYTFTDYYIHWVRQATGQGL
EWMGWMNPNSGNTGYAQKFQGRVTMTRDTSISTAYMELSSLRSED
TAYYCARGQGYTHSWGQGTMVTVSSASTKGPSVFPLAPSSKSTSG
GTAALGCLVKDYFPEPVTVSWNSGALTSGVHTFPALQSSGLYSLS
SVVTVPSSSLGTQTYICNVNHKPSNTKVDKRVEPKSCDKTHTCPP
CPAPEFEGGPSVFLFPPKPKDTLMISRTPEVTCVVVDVSHEDPEV
KFNWYVDGVEVHNAKTKPREEQYNSTYRVVSVLTVLHQDWLNGKE
YKCKVSNKALPASIEKTISKAKGQPREPQVCTLPPSREEMTKNQV
SLSCAKGFYPSDIAEWESNGQPENNYKTTPPVLDSDGSFFLVSKL
TVDKSRWQQGNVFSCSVMHEALHNHYTQKSLSLSPGK

Amino acid sequence of the light chain of low affinity anti-EGFR binding arm (RAA22) in “EGFR-cMET™” antibody (SEQ ID NO: 61):

Following substitutions are underlined:

S121C and C214V, wherein numbering is according to EU index

QSALTQPRSVSGSPGQSVTISCTGTSSDVGGYNYVSWYQQHPGKA
PKLMIYDVSKRPSGVPDRFSGSKSGNTASLTISGLQAEDEADYYC
SSYTSSDTLEIFGGGTKLTVLGQPKAAPSVTLFPPCSEELQANKA
TLCLISDFYPGATVAWKADSSPVKAGVETTTPSKQSNNKYAASSY
LSLTPEQWKSHRSYSCQVTHEGSTVEKTVAPTEVS
Amino acid sequence of the light chain of
anti-cMet binding arm in “EGFR-cMET TM”
antibody (SEQ ID NO: 62):
DIQMTQSPSTLSASVGDRVTITCRASEGIYHWLAWYQQKPGKAPK
LLIYKASSLASGVPSRFSGSGSGTEFTLTISSLQPDDFATYYCQQ
YSNYPPTFGGGTKLEIKRTVAAPSVFIFPPSDEQLKSGTASVVCL
LNNFYPREAKVQWKVDNALQSGNSQESVTEQDSKDSTYSLSSTLT
LSKADYEKHKYACEVTHQGLSSPVTKSFNRGEC

Amino acid sequence of a human immunoglobulin G1 CH region modified to include “knob” mutations, a cysteine to form a stabilizing disulfide bridge, without a cysteine insertion and with the TM (SEQ ID NO: 63):

Following substitutions are underlined:

Triple mutation (TM; L234F, L235E and P331S); “Knob” mutation (T366W); interchain cysteine mutations (F126C and C219V); stabilizing cysteine mutation (S354C), where numbering of residues is according to EU index.

ASTKGPSVCPLAPSSKSTSGGTAALGCLVKDYFPEPVTVSWNSGA
LTSGVHTFPALQSSGLYSLSSVVTVPSSSLGTQTYICNVNHKPSN
TKDKRVEPKSVDKTHTCPPCPAPEFEGGPSVFLFPPKPKDTLMIS
RTPEVTCVVVDVSHEDPEVKFNWYVDGVEVHNAKTKPREEQYNST
YRVVSVLTVLHQDWLNGKEYKCKVSNKALPASIEKTISKAKGQPR
EPQVYTLPPCREEMTKNQVSLWCLKGFYPSDIAEWESNGQPENNY
KTTPPVLDSDGSFFLYSKLTVDKSRWQQGNVFSCSVMHEALHNHY
TQKSLSLSPGK

Amino acid sequence of a human immunoglobulin G1 CH region modified to include “Hole” mutations, a cysteine to form a stabilizing disulfide bridge, without a cysteine insertion and with the TM (SEQ ID NO: 64):

Following substitutions are underlined:

Triple mutation (TM; L234F, L235E and P331S); “Hole” mutations (T366S, L368A, and Y407V); and stabilizing cysteine mutation (Y349C), where numbering of residues is according to EU index.

ASTKGPSVFPLAPSSKSTSGGTAALGCLKDYFPEPVTVSWNSGAL
TSGVHTFPALQSSGLYSLSSVVTVPSSSLGTQTYICNVNHKPSNT
KDKRVEPKSCDKTHTCPPCPAPEFEGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPASIEKTISKAKGQPRE
PQVCTLPPSREEMTKNQVSLSCAKGFYPSDIAEWESNGQPENNYK
TTPPVLDSDGSFFLVSKLTVDKSRWQQGNVFSCSVMHEALHNHYT
QKSLSLSPGK
Amino acid sequence of a human immunoglobulin
lambda constant region modified to include
S121C and C214V substitutions (SEQ ID NO: 65):
GQPKAAPSVTLFPPCSEELQANKATLVCLISDFYPGATVAWKADS
SPVKAGVETTTPSKQSNNKYAASSYLSLTPEQWKSHRSYSCQVTH
EGSTVEKTVAPTEVS
Amino acid sequence of the human EGFR
extracellular domain (SEQ ID NO: 68):
LEEKKVCQGTSNKLTQLGTFEDHFLSLQRMFNNCEVVLGNLEITY
VQRNYDLSFLKTIQEVAGYVLIALNTVERIPLENLQIIRGNMYYE
NSYALALSNYDANKTGLKELPMRNLQEILHGARFSNNPALCNVES
IQWRDIVSSDFLSNMSMDFQNHLGSCQKCDPSCPNGSCWGAGEEN
CQKLTKIICAQQCSGRCRGKSPSDCCHNQCAAGCTGPRESDCLVC
RKFRDEATCKDTCPPLMLYNPTTYQMDVNPEGKYSFGATCVKKCP
RNYVVTDHGSCVRACGADSYEMEEDGVRKCKKCEGPCRKVCNGIG
IGEFKDSLSINATNIKHFKNCTSISGDLHILPVAFRGDSFTHTPP
LDPQELDILKTVKEITGFLLIQAWPENRTDLHAFENLEIIRGRTK
QHGQFSLAVSLNITSLGLRSLKEISDGDVIISGNKNLCYANTINW
KKLFGTSGQKTKIISNRGENSCKATGQVCHALCSPEGCWGPEPRD
CVSCRNVSRGRECVDKCNLLEGEPREFVENSECIQCHPECLPQAM
NITCTGRGPDNCIQCAHYIDGPHCVKTCPAGVMGENNTLWKYADA
GHVCHLCHPNCTYGCTGPGLEGCPTNGPKIPS
Amino acid sequence of the cynomolgus monkey
EGFR extracellular domain (SEQ ID NO: 69):
LEEKKVCQGTSNKLTQLGTFEDHFLSLQRMFNNCEVVLGNLEITY
VQRNYDLSFLKTIQEVAGYVLIALNTVERIPLENLQIIRGNMYYE
NSYALALSNYDANKTGLKELPMRNLQEILHGARFSNNPALCNVES
IQWRDIVSSEFLSNMSMDFQNHLGSCQKCDPSCPNGSCWGAGEEN
CQKLTKIICAQQCSGRCRGKSPSDCCHNQCAAGCTGPRESDCLVC
RKFRDEATCKDTCPPLMLYNPTTYQMDVNPEGKYSFGATCVKKCP
RNYVVTDHGSCVRACGADSYEMEEDGVRKCKKCEGPCRKVCNGIG
IGEFKDTLSINATNIKHFKNCTSISGDLHILPVAFRGDSFTHTPP
LDPQELDILKTVKEITGFLLIQAWPENRTDLHAFENLEIIRGRTK
QHGQFSLAVSLNITSLGLRSLKEISDGDVIISGNKNLCYANTINW
KKLFGTSSQKTKIISNRGENSCKATGQVCHALCSPEGCWGPEPRD
CVSCQNVSRGRECVDKCNILEGEPREFVENSECIQCHPECLPQVM
NITCTGRGPDNCIQCAHYIDGPHCVKTCPAGVMGENNTLWKYADA
GHVCHLCHPNCTYGCTGPGLEGCARNGPKIPS
Amino acid sequence of the human cMET
extracellular domain (SEQ ID NO: 70):
ECKEALAKSEMNVNMKYQLPNFTAETPIQNVILHEHHIFLGATNY
IYVLNEEDLQKVAEYKTGPVLEHPDCFPCQDCSSKANLSGGVWKD
NINMALVDTYYDDQLISCGSVNRGTCQRHVFPHNHTADIQSEVHC
IFSPQIEEPSQCPDCVVSALGAKVLSSVKDRFINFFVGNTINSSY
FPDHPLHSISVRRLKETKDGFMFLTDQSYIDVLPEFRDSYPIKYV
HAFESNNFIYFLTVQRETLDAQTFHTRIIRFCSINSGLHSYMEMP
LECILTEKRKKRSTKKEVFNILQAAYVSKPGAQLARQIGASLNDD
ILFGVFAQSKPDSAEPMDRSAMCAFPIKYVNDFFNKIVNKNNVRC
LQHFYGPNHEHCFNRTLLRNSSGCEARRDEYRTEFTTALQRVDLF
MGQFSEVLLTSISTFIKGDLTIANLGTSEGRFMQVVVSRSGPSTP
HVNFLLDSHPVSPEVIVEHTLNQNGYTLITGKKITKIPLNGLGCR
HFQSCSQCLSAPPFVQCGWCHDKCVRSEECLSGTWTQQICLPAIY
KVFPNSAPLEGGTRLTICGWDFGFRRNNKFDLKKTRVLLGNESCT
LTLSESTMNTLKCTVGPAMNKHFNMSIIISNGHGTTQYSTFSYVD
PVITSISPKYGPMAGGTLLTLTGNYLNSGNSRHISIGGKTCTLKS
VSNSILECYTPAQTISTEFAVKLKIDLANRETSIFSYREDPIVYE
IHPTKSFISGGSTITGVGKNLNSVSVPRMVINVHEAGRNFTVACQ
HRSNSEIICCTTPSLQQLNLQLPLKTKAFFMLDGILSKYFDLIYV
HNPVFKPFEKPVMISMGNENVLEIKGNDIDPEAKGEVLKVGNKSC
ENIHLHSEALCTVPNDLLKLNSELNIEWKQAISSTVLGKVIVQPD
QNFT
Amino acid sequence of the cynomolgus monkey
cMET extracellular domain (SEQ ID NO: 71):
ECKEALAKSEMNVNMKYQLPNFTAETAIQNVILHEHHIFLGATNY
IYVLNEEDLQKVAEYKTGPVLEHPDCFPCQDCSSKANLSGGVWKD
NINMALVDTYYDDQLISCGSVNRGTCQRHVFPHNHTADIQSEVHC
IFSPQIEEPNQCPDCVVSALGAKVLSSVKDRFINFFVGNTINSSY
FPHHPLHSISVRRLKETKDGFMFLTDQSYIDVLPEFRDSYPIKYI
HAFESNNFIYFLTVQRETLNAQTFHTRIIRFCSLNSGLHSYMEMP
LECILTEKRKKRSTKKEVFNILQAAYVSKPGAQLARQIGASLNDD
ILFGVFAQSKPDSAEPMDRSAMCAFPIKYVNDFFNKIVNKNNVRC
LQHFYGPNHEHCFNRTLLRNSSGCEARRDEYRAEFTTALQRVDLF
MGQFSEVLLTSISTFVKGDLTIANLGTSEGRFMQVVVSRSGPSTP
HVNFLLDSHPVSPEVIVEHPLNQNGYTLVTGKKITKIPLNGLGCR
HFQSCSQCLSAPPFVQCGWCHDKCVRSEECPSGTWTQQICLPAIY
KVFPTSAPLEGGTRLTICGWDFGFRRNNKFDLKKTRVLLGNESCT
LTLSESTMNTLKCTVGPAMNKHFNMSIIISNGHGTTQYSTFSYVD
PIITSISPKYGPMAGGTLLTLTGNYLNSGNSRHISIGGKTCTLKS
VSNSILECYTPAQTISTEFAKLKIDLANRETSIFSYREDPIVYEI
HPTKSFISGGSTITGVGKNLHSVSVPRMVINVHEAGRNFTVACQH
RSNSEIICCTTPSLQQLNLQLPLKTKAFFMLDGILSKYFDLIYVH
NPVFKPFEKPVMISMGNENVLEIKGNDIDPEAKGEVLKVGNKSCE
NIHLHSEALCTVPNDLLKLNSELNIEWKQAISSTVLGKVIVQPDQ
NFT

1-34. (canceled)

35. A method of treating cancer in a human patient in need of such a treatment, comprising administering to the human patient a first amount of an EGFR TKI, and a second amount of an anti-EGFR/cMET antibody molecule, where the first amount and the second amount together comprise a therapeutically effective amount, wherein the anti-EGFR/cMET antibody molecule comprises an EGFR binding domain and a cMET binding domain, wherein the EGFR binding domain comprises:
a. a heavy chain variable (VH) region comprising the following complementarity determining regions (CDRs):
i. HCDR1 having the amino acid sequence of SEQ ID NO: 1;
ii. HCDR2 having the amino acid sequence of SEQ ID NO: 2; and
iii. HCDR3 having the amino acid sequence of SEQ ID NO: 3; and
b. a light chain variable (VL) region comprising the following CDRs:
i. LCDR1 having the amino acid sequence of SEQ ID NO: 4;
ii. LCDR2 having the amino acid sequence of SEQ ID NO: 5; and
LCDR3 having the amino acid sequence of SEQ ID NO: 6.

36. (canceled)

37. The method according to claim 35 wherein the administration of the EGFR TKI and the anti-EGFR/cMET antibody molecule is separate, sequential, or simultaneous.

38. The method according to claim 35, wherein the EGFR TKI is a compound of Formula (V): 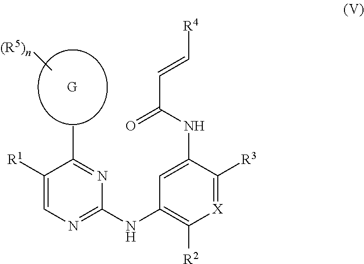
wherein:
G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;
R¹ is selected from hydrogen, fluoro, chloro, methyl and cyano;
R² is selected from methoxy, trifluoromethoxy, ethoxy, 2,2,2-trifluoroethoxy and methyl;
R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;
R⁴ is selected from hydrogen, 1-piperidinomethyl and N,N-dimethylaminomethyl;
R⁵ is independently selected from methyl, ethyl, propyl, 2,2-difluoroethyl, 2,2,2-trifluoroethyl, fluoro, chloro and cyclopropyl;
X is CH or N; and
n is 0, 1 or 2;
or a pharmaceutically acceptable salt thereof.

39. The method according to claim 38 wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from [2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

40. The method according to claim 35, wherein the EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, afatinib or a pharmaceutically acceptable salt thereof, CX-101 or a pharmaceutically acceptable salt thereof, HS-10296 or a pharmaceutically acceptable salt thereof, BPI-7711 or a pharmaceutically acceptable salt thereof, dacomitinib or a pharmaceutically acceptable salt thereof, icotinib or a pharmaceutically acceptable salt thereof, gefitinib or a pharmaceutically acceptable salt thereof and erlotinib or a pharmaceutically acceptable salt thereof.

41. The method according to claim 40, wherein the EGFR TKI is osimertinib or a pharmaceutically acceptable salt thereof.

42. The method according to claim 35, wherein the anti-EGFR binding domain comprises a VH region comprising an amino acid sequence having at least 70%, at least 80%, at least 90%, or at least 95% sequence identity to the amino acid sequence of SEQ ID NO: 16; and a VL region comprising an amino acid sequence having at least 70%, at least 80%, at least 90%, or at least 95% sequence identity to the amino acid sequence of SEQ ID NO: 20.

43. The method according to claim 35, wherein the wherein the anti-cMET binding domain comprises:
a. a heavy chain variable (VH) region comprising the following complementarity determining regions (CDRs):
i. HCDR1 having the amino acid sequence of SEQ ID NO: 24;
ii. HCDR2 having the amino acid sequence of SEQ ID NO: 25; and
iii. HCDR3 having the amino acid sequence of SEQ ID NO: 26; and
b. a light chain variable (VL) region comprising the following CDRs:
i. LCDR1 having the amino acid sequence of SEQ ID NO: 27;
ii. LCDR2 having the amino acid sequence of SEQ ID NO: 28; and
iii. LCDR3 having the amino acid sequence of SEQ ID NO: 29.

44. The method according to claim 43, wherein the cMET binding domain comprises: a VH region comprising an amino acid sequence having at least 70%, at least 80%, at least 90%, or at least 95% sequence identity to the amino acid sequence of SEQ ID NO: 38; and a VL region comprising an amino acid sequence having at least 70%, at least 80%, at least 90%, or at least 95% sequence identity to the amino acid sequence of SEQ ID NO: 40.

45. The method according to claim 35, wherein the antibody molecule comprises:
a. a first heavy chain, wherein the first heavy chain comprises the VH region of the anti-EGFR binding domain, and a first heavy chain constant (CH) region or a fragment thereof;
b. a first light chain, wherein the first light chain comprises the VL region of the anti-EGFR binding domain, and a first light chain constant (CL) region or a fragment thereof,
c. a second heavy chain, wherein the second heavy chain comprises the VH region of the anti-cMET binding domain, and a second heavy chain constant (CH) region or a fragment thereof; and
d. a second light chain, wherein the second light chain comprises the VL region of the anti-cMET binding domain, and a second light chain constant (CL) region or a fragment thereof.

46. The method according to claim 45, wherein the first and second CH region comprises an amino acid sequence having at least 70%, at least 80%, at least 90%, or at least 95% sequence identity to the amino acid sequence of SEQ ID NO: 42.

47. The method according to claim 45, wherein the first and/or second CH region comprise a mutation to reduce or abrogate binding of the antibody molecule to one of more Fcγ receptors.

48. The method according to claim 47, wherein the first and/or second CH region comprise a phenylalanine at position 234, glutamic acid at position 235, and serine at position 331, wherein the numbering of the constant region is as per the EU index.

49. The method according claim 45, wherein the first CH region comprises an amino acid sequence having at least 90%, 91%, 92%, 93%, 94%, 95%, 96%, 97%, 98%, 99%, or 100% sequence identity to the sequence set forth in SEQ ID NO: 63; and the second CH region comprises an amino acid sequence having at least 90%, 91%, 92%, 93%, 94%, 95%, 96%, 97%, 98%, 99%, or 100% sequence identity to the sequence set forth in SEQ ID NO: 64, and wherein the first CL region comprises an amino acid sequence having at least 90%, 91%, 92%, 93%, 94%, 95%, 96%, 97%, 98%, 99%, or 100% sequence identity to the sequence set forth in SEQ ID NO: 65; and the second CL region comprises an amino acid sequence having at least 90%, 91%, 92%, 93%, 94%, 95%, 96%, 97%, 98%, 99%, or 100% sequence identity to the sequence set forth in SEQ ID NO: 47.

50. The method according to claim 45, wherein the first heavy chain comprises an amino acid sequence having the sequence set forth in SEQ ID NO: 59; the second heavy chain comprises an amino acid sequence having the sequence set forth in SEQ ID NO: 60; the first light chain comprises an amino acid sequence having the sequence set forth in SEQ ID NO: 61; and the second light chain comprises an amino acid sequence having the sequence set forth in SEQ ID NO: 62.

51. The method according to claim 35, wherein the antibody molecule is conjugated to a drug.

52. The method according to claim 51, wherein the drug comprises a cytotoxin, a radioisotope, an immunomodulator, a cytokine, a lymphokine, a chemokine, a growth factor, a tumor necrosis factor, a hormone, a hormone antagonist, an enzyme, an oligonucleotide, a DNA, an RNA, an siRNA, an RNAi, a microRNA, a photoactive therapeutic agent, an anti-angiogenic agent, a pro-apoptotic agent, a peptide, a lipid, a carbohydrate, a chelating agent, or combinations thereof.

53. The method according to claim 51, wherein the drug is a topoisomerase I inhibitor having formula A*

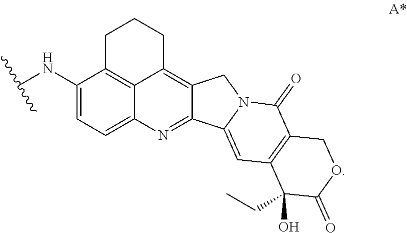 [SMILES: *Nc1ccc2nc3c(c4c2c1CCC4)Cn1c-3cc2c(c1=O)COC(=O)[C@]2(O)CC]

54. The method according to claim 51, wherein the topoisomerase I inhibitor has formula 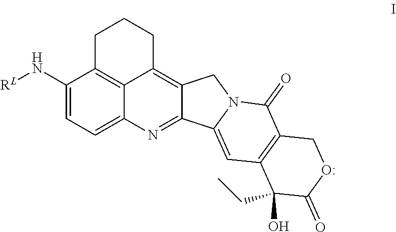
or a salt or solvates thereof, wherein R^{L} is a linker for connection to the antibody molecule, optionally wherein said linker is selected from: 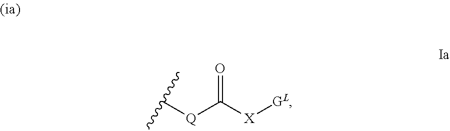
wherein
Q is: 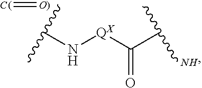
where Q^{X} is such that Q is an amino-acid residue, a dipeptide residue, a tripeptide residue or a tetrapeptide residue;
X is: 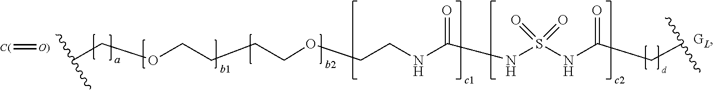
where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0);
G^{L} is a linker for connecting to the antibody molecule; or 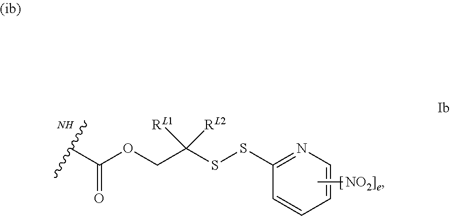
wherein
R^{L1} and R^{L2} are independently selected from H and methyl, or together with the carbon atom to which they are bound form a cyclopropylene or cyclobutylene group; and
e is 0 or 1.

55. The method according to claim 51, wherein the antibody molecule is conjugated to a topoisomerase I inhibitor having the following formula:

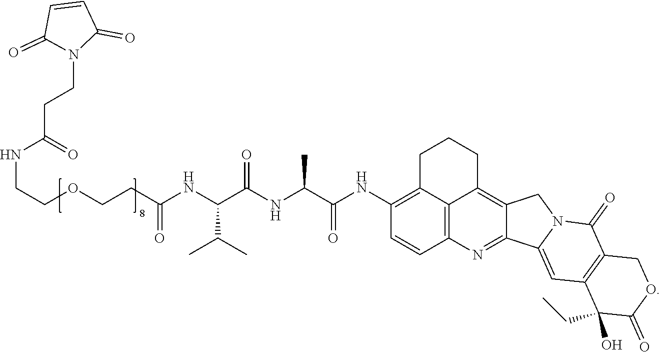

56. The method according to claim 51, wherein the anti-EGFR/cMET antibody molecule comprises an EGFR binding domain and a cMET binding domain,
wherein the EGFR binding domain comprises:
a. a heavy chain variable (VH) region having the sequence set forth in SEQ ID NO: 16; and
b. a light chain variable (VL) region having the sequence set forth in SEQ ID NO: 20;
wherein the cMET binding domain comprises:
a. a heavy chain variable (VH) region having the sequence set forth in SEQ ID NO: 38; and
b. a light chain variable (VL) region having the sequence set forth in SEQ ID NO: 40; and
wherein the antibody molecule is conjugated to a topoisomerase I inhibitor having the following formula: 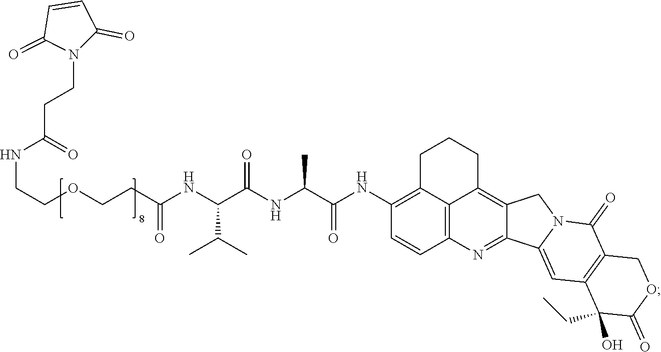
and
wherein the EGFR TKI is osimertinib, or a pharmaceutically acceptable salt thereof.

57. The method according to claim 35, wherein the cancer is non-small cell lung cancer, optionally wherein the non-small cell lung cancer is an EGFR mutation-positive non-small cell lung cancer.

58. The method according to claim 57, wherein the EGFR mutation-positive non-small cell lung cancer comprises i) one or more deletions in exon 19 in the EGFR gene and/or a L858R mutation; and/or ii) a T790M mutation, a mutation in exon 20 in the EGFR gene, or both.

59. The method according to claim 35, wherein the human patient is an EGFR TKI-naïve human patient.

60. The method according to claim 35, wherein the human patient's disease has progressed during or after previous EGFR TKI treatment, optionally wherein the EGFR TKI is osimertinib or a pharmaceutically acceptable salt thereof and the human patient's disease has progressed during or after previous treatment with a different EGFR TKI.


SEQUENCE LISTING (71 sequences)

SEQ ID NO: 1 - protein, 5 aa, synthetic construct
DNDFS

SEQ ID NO: 2 - protein, 17 aa, synthetic construct
AIVAVFRTETYAQKFQD

SEQ ID NO: 3 - protein, 15 aa, synthetic construct
RLMSAISGPGAPLLM

SEQ ID NO: 4 - protein, 14 aa, synthetic construct
TGTSSDVGGYNYVS

SEQ ID NO: 5 - protein, 7 aa, synthetic construct
DVSKRPS

SEQ ID NO: 6 - protein, 11 aa, synthetic construct
SSYTSSDTLEI

SEQ ID NO: 7 - protein, 17 aa, synthetic construct
AIVAVVRTETYAQKFQD

SEQ ID NO: 8 - protein, 30 aa, synthetic construct
QVQLVQSGAEVKKPGSSVKVSCKASGGTFS

SEQ ID NO: 9 - protein, 14 aa, synthetic construct
WVRQAPGQGLEWMG

SEQ ID NO: 10 - protein, 32 aa, synthetic construct
RVKITADISTRTTYMELSSLRSEDTAVYYCAR

SEQ ID NO: 11 - protein, 11 aa, synthetic construct
WGQGTLVTVSS

SEQ ID NO: 12 - protein, 22 aa, synthetic construct
QSALTQPRSVSGSPGQSVTISC

SEQ ID NO: 13 - protein, 15 aa, synthetic construct
WYQQHPGKAPKLMIY

SEQ ID NO: 14 - protein, 32 aa, synthetic construct
GVPDRFSGSKSGNTASLTISGLQAEDEADYYC

SEQ ID NO: 15 - protein, 10 aa, synthetic construct
FGGGTKLTVL

SEQ ID NO: 16 - protein, 124 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSS

SEQ ID NO: 17 - DNA, 372 bp, synthetic construct
  source 1..372: mol_type = other DNA
caggtgcagctggtgcagtctggggccgaagtgaagaaacccggcagcagcgtgaaggtgtcctgtaaagccagcggcggcaccttcagcgacaacgactttagctgggtccgacaggcccctggacagggcctggaatggatgggagccatcgtggccgtgttccggacagagacatacgcccagaaattccaggacagagtgaaaatcaccgccgacatcagcaccagaaccacctacatggaactgagcagcctgagaagcgaggacaccgccgtgtactactgcgccagacggctgatgtctgccatctctggacctggcgctcctctgctcatgtggggacagggaacactggtcaccgtgtccagc

SEQ ID NO: 18 - protein, 124 aa, synthetic construct
QVQLVQSGAEVKKPGSSVKVSCKASGGTFSDNDFSWVRQAPGQGLEWMGAIVAVVRTETYAQKFQDRVKITADISTRTTYMELSSLRSEDTAVYYCARRLMSAISGPGAPLLMWGQGTLVTVSS

SEQ ID NO: 19 - DNA, 372 bp, synthetic construct
  source 1..372: mol_type = other DNA
caggtgcagctggtgcagtctggggccgaagtgaagaaacccggcagcagcgtgaaggtgtcctgtaaagccagcggcggcaccttcagcgacaacgactttagctgggtccgacaggcccctggacagggcctggaatggatgggagccatcgtggccgtggtccggacagagacatacgcccagaaattccaggacagagtgaaaatcaccgccgacatcagcaccagaaccacctacatggaactgagcagcctgagaagcgaggacaccgccgtgtactactgcgccagacggctgatgtctgccatctctggacctggcgctcctctgctcatgtggggacagggaacactggtcaccgtgtccagc

SEQ ID NO: 20 - protein, 111 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVL

SEQ ID NO: 21 - DNA, 333 bp, synthetic construct
  source 1..333: mol_type = other DNA
cagtctgccctgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtaagcggccctcaggggtccctgatcgcttctctggctccaagtctggcaacacggcctccctgaccatctctgggctccaggctgaggacgaggctgattattactgcagttcatatacaagcagcgacactctcgaaatattcggcggagggaccaagctgaccgtccta

SEQ ID NO: 22 - protein, 111 aa, synthetic construct
QSALTQPRSVSGSPGQSVTISCTGTSSDVGGYNYVSWYQQHPGKAPKLMIYDVSERPSGVPDRFSGSKSGNTASLTISGLQAEDEADYYCFSYTSSDTLEIFGGGTKLTVL

SEQ ID NO: 23 - DNA, 333 bp, synthetic construct
  source 1..333: mol_type = other DNA
cagtctgccctgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtgaacggccctcaggggtccctgatcgcttctctggctccaagtctggcaacacggcctccctgaccatctctgggctccaggctgaggacgaggctgattattactgcttctcatatacaagcagcgacactctcgaaatattcggcggagggaccaagctgaccgtccta

SEQ ID NO: 24 - protein, 5 aa, synthetic construct
DYYIH

SEQ ID NO: 25 - protein, 17 aa, synthetic construct
WMNPNSGNTGYAQKFQG

SEQ ID NO: 26 - protein, 7 aa, synthetic construct
GQGYTHS

SEQ ID NO: 27 - protein, 11 aa, synthetic construct
RASEGIYHWLA

SEQ ID NO: 28 - protein, 7 aa, synthetic construct
KASSLAS

SEQ ID NO: 29 - protein, 9 aa, synthetic construct
QQYSNYPPT

SEQ ID NO: 30 - protein, 30 aa, synthetic construct
QVQLVQSGAEVKKPGASVKVSCKASGYTFT

SEQ ID NO: 31 - protein, 14 aa, synthetic construct
WVRQATGQGLEWMG

SEQ ID NO: 32 - protein, 32 aa, synthetic construct
RVTMTRDTSISTAYMELSSLRSEDTAVYYCAR

SEQ ID NO: 33 - protein, 11 aa, synthetic construct
WGQGTMVTVSS

SEQ ID NO: 34 - protein, 23 aa, synthetic construct
DIQMTQSPSTLSASVGDRVTITC

SEQ ID NO: 35 - protein, 15 aa, synthetic construct
WYQQKPGKAPKLLIY

SEQ ID NO: 36 - protein, 32 aa, synthetic construct
GVPSRFSGSGSGTEFTLTISSLQPDDFATYYC

SEQ ID NO: 37 - protein, 10 aa, synthetic construct
FGGGTKLEIK

SEQ ID NO: 38 - protein, 116 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSS

SEQ ID NO: 39 - DNA, 348 bp, synthetic construct
  source 1..348: mol_type = other DNA
caagtgcagctggtgcagtctggcgccgaagtgaagaaacctggcgccagcgtgaaggtcagctgcaaggccagcggctacaccttcaccgactactacatccactgggtccgccaggccacaggccagggactggaatggatgggctggatgaaccccaacagcggcaacaccggctacgcccagaaattccagggcagagtgaccatgacccgggacaccagcatcagcaccgcctacatggaactgagcagcctgcggagcgaggacaccgccgtgtactactgtgccagaggccagggctacacccacagctggggccagggcaccatggtcacagtgtccagc

SEQ ID NO: 40 - protein, 107 aa, Synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIK

SEQ ID NO: 41 - DNA, 321 bp, synthetic construct
  source 1..321: mol_type = other DNA
gacatccagatgacccagagccccagcaccctgagcgccagcgtcggcgacagagtgaccatcacctgtcgggccagcgagggcatctaccactggctggcctggtatcagcagaagcccggcaaggcccccaagctgctgatctacaaggccagcagcctggccagcggagtccctagcagattttctggcagcggcagcggcaccgagttcaccctgaccatcagcagcctgcagcccgacgacttcgccacctactactgccagcagtacagcaactacccccccaccttcggcggaggcaccaagctggaaatcaag

SEQ ID NO: 42 - protein, 330 aa, synthetic construct [sequence omitted]

SEQ ID NO: 43 - protein, 330 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 329 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 331 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 107 aa, synthetic construct [sequence omitted]

SEQ ID NO: 48 - protein, 107 aa, synthetic construct
RTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEV

SEQ ID NO: 49 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 50 - protein, 446 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 445 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 214 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEV

SEQ ID NO: 53 - protein, 455 aa, synthetic construct
QVQLVQSGAEVKKPGSSVKVSCKASGGTFSDNDFSWVRQAPGQGLEWMGAIVAVVRTETYAQKFQDRVKITADISTRTTYMELSSLRSEDTAVYYCARRLMSAISGPGAPLLMWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 454 aa, synthetic construct
QVQLVQSGAEVKKPGSSVKVSCKASGGTFSDNDFSWVRQAPGQGLEWMGAIVAVVRTETYAQKFQDRVKITADISTRTTYMELSSLRSEDTAVYYCARRLMSAISGPGAPLLMWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 55 - protein, 217 aa, synthetic construct
QSALTQPRSVSGSPGQSVTISCTGTSSDVGGYNYVSWYQQHPGKAPKLMIYDVSERPSGVPDRFSGSKSGNTASLTISGLQAEDEADYYCFSYTSSDTLEIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 455 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 455 aa, synthetic construct; identical to SEQ ID NO: 56

SEQ ID NO: 58 - protein, 217 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - protein, 454 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 446 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 217 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 62 - protein, 214 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 330 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 65 - protein, 106 aa, synthetic construct
GQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 66 - protein, 7 aa, synthetic construct
DVSERPS

SEQ ID NO: 67 - protein, 11 aa, synthetic construct
FSYTSSDTLEI

SEQ ID NO: 68 - protein, 621 aa, synthetic construct [sequence omitted]

SEQ ID NO: 69 - protein, 621 aa, synthetic construct
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPS

SEQ ID NO: 70 - protein, 908 aa, synthetic construct
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 71 - protein, 908 aa, synthetic construct
ECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT